SEQUENCE LISTING (102 sequences)

SEQ ID NO: 1 - DNA, 24 bp, Artificial Sequence
  Synthetic oligonucleotide primer.
acttcgaggtcactttccagcagt

SEQ ID NO: 2 - DNA, 24 bp, Artificial Sequence
  Synthetic oligonucleotide primer.
taagaccaccctgctgtggaaaca

SEQ ID NO: 3 - DNA, 24 bp, Artificial Sequence
  Synthetic oligonucleotide primer.
accatccttctcaggaatcgccaa

SEQ ID NO: 4 - DNA, 24 bp, Artificial Sequence
  Synthetic oligonucleotide primer.
tctctttccaccttcaatcggcct

SEQ ID NO: 5 - DNA, 24 bp, Artificial Sequence
  Synthetic oligonucleotide primer.
agcacatctacgatggagcgtctt

SEQ ID NO: 6 - DNA, 22 bp, Artificial Sequence
  Synthetic oligonucleotide primer.
gccacttctcggaggaggtgct

SEQ ID NO: 7 - DNA, 24 bp, Artificial Sequence
  Synthetic oligonucleotide primer.
atggtgagaaggcaaccaaagtgc

SEQ ID NO: 8 - DNA, 24 bp, Artificial Sequence
  Synthetic oligonucleotide primer.
atgcacttgtccggctagagatga

SEQ ID NO: 9 - DNA, 23 bp, Artificial Sequence
  Synthetic oligonucleotide primer.
aaccggctcaagaccaccaagta

SEQ ID NO: 10 - DNA, 24 bp, Artificial Sequence
  Synthetic oligonucleotide primer.
cttccagctatggaagaagaagcg

SEQ ID NO: 11 - DNA, 24 bp, Artificial Sequence
  Synthetic oligonucleotide primer.
agctagaggaaccagacctcatca

SEQ ID NO: 12 - DNA, 20 bp, Artificial Sequence
  Synthetic oligonucleotide primer.
tcgctgaggacaggaaacct

SEQ ID NO: 13 - DNA, 24 bp, Artificial Sequence
  Synthetic oligonucleotide primer.
taaagagcgcaggacctcctcaaa

SEQ ID NO: 14 - DNA, 24 bp, Artificial Sequence
  Synthetic oligonucleotide primer.
gcggcatttggacaaagcagatca

SEQ ID NO: 15 - DNA, 22 bp, Artificial Sequence
  Synthetic oligonucleotide primer.
atgggataacgcggccatggtg

SEQ ID NO: 16 - DNA, 24 bp, Artificial Sequence
  Synthetic oligonucleotide primer.
acaacaacaactccaagcacaccg

SEQ ID NO: 17 - DNA, 24 bp, Artificial Sequence
  Synthetic oligonucleotide primer.
gccacgtttccctgccaattatgt

SEQ ID NO: 18 - DNA, 24 bp, Artificial Sequence
  Synthetic oligonucleotide primer.
tcccaacgatgggtaacgtcagtt

SEQ ID NO: 19 - DNA, 24 bp, Artificial Sequence
  Synthetic oligonucleotide primer.
tacccatcagcaggtgtcctcttt

SEQ ID NO: 20 - DNA, 24 bp, Artificial Sequence
  Synthetic oligonucleotide primer.
tgtaccaaccggaagacgacaaca

SEQ ID NO: 21 - DNA, 24 bp, Artificial Sequence
  Synthetic oligonucleotide primer.
ccaaagtcccgctaagattctcca

SEQ ID NO: 22 - DNA, 20 bp, Artificial Sequence
  Synthetic oligonucleotide primer.
gagccgcccgaatacgagtt

SEQ ID NO: 23 - DNA, 24 bp, Artificial Sequence
  Synthetic oligonucleotide primer.
agttactgtggatgagggtgatgc

SEQ ID NO: 24 - DNA, 24 bp, Artificial Sequence
  Synthetic oligonucleotide primer.
tggagaaggagaagctggagcaaa

SEQ ID NO: 25 - DNA, 24 bp, Artificial Sequence
  Synthetic oligonucleotide primer.
cagaactgtgttctcttccaccca

SEQ ID NO: 26 - DNA, 24 bp, Artificial Sequence
  Synthetic oligonucleotide primer.
catattggccagtctggtctcgaa

SEQ ID NO: 27 - DNA, 20 bp, Artificial Sequence
  Synthetic oligonucleotide primer.
agctatgcccggacctccat

SEQ ID NO: 28 - DNA, 24 bp, Artificial Sequence
  Synthetic oligonucleotide primer.
cctgtgtccttccatttcctgtct

SEQ ID NO: 29 - DNA, 18 bp, Artificial Sequence
  Synthetic oligonucleotide primer.
cccccggcggcaatagca

SEQ ID NO: 30 - DNA, 22 bp, Artificial Sequence
  Synthetic oligonucleotide primer.
gtggtctcctccgacttcaaca

SEQ ID NO: 31 - DNA, 22 bp, Artificial Sequence
  Synthetic oligonucleotide primer.
gtcattgctgaaaccgagaatg

SEQ ID NO: 32 - DNA, 22 bp, Artificial Sequence
  Synthetic oligonucleotide primer.
gaaccagaggggagagacagag

SEQ ID NO: 33 - DNA, 20 bp, Artificial Sequence
  Synthetic oligonucleotide primer.
tggctacagctgcactgccg

SEQ ID NO: 34 - DNA, 22 bp, Artificial Sequence
  Synthetic oligonucleotide primer.
acagagtttattgaggtgcccc

SEQ ID NO: 35 - DNA, 22 bp, Artificial Sequence
  Synthetic oligonucleotide primer.
ggcagcggaagaggatgctgaa

SEQ ID NO: 36 - DNA, 24 bp, Artificial Sequence
  Synthetic oligonucleotide primer.
tattgccttccacacggatgaggt

SEQ ID NO: 37 - DNA, 24 bp, Artificial Sequence
  Synthetic oligonucleotide primer.
gtttgccagtgttgctgagatgct

SEQ ID NO: 38 - DNA, 24 bp, Artificial Sequence
  Synthetic oligonucleotide primer.
tgtgtttgtgtcaatcccagcgag

SEQ ID NO: 39 - DNA, 24 bp, Artificial Sequence
  Synthetic oligonucleotide primer.
caggaaagtctgcggcaatgttgt

SEQ ID NO: 40 - DNA, 24 bp, Artificial Sequence
  Synthetic oligonucleotide primer.
agtcgctgtccacttcggtccact

SEQ ID NO: 41 - DNA, 23 bp, Artificial Sequence
  Synthetic oligonucleotide primer.
ggagcagtggttgtacagaggcg

SEQ ID NO: 42 - DNA, 24 bp, Artificial Sequence
  Synthetic oligonucleotide primer.
gaaggtgcatgcgctgaatgatgt

SEQ ID NO: 43 - DNA, 24 bp, Artificial Sequence
  Synthetic oligonucleotide primer.
agccagacccagtactatgccaat

SEQ ID NO: 44 - DNA, 25 bp, Artificial Sequence
  Synthetic oligonucleotide primer.
tgtagtcctcccataggtccctgaa

SEQ ID NO: 45 - DNA, 22 bp, Artificial Sequence
  Synthetic oligonucleotide primer.
cggcggttctggaaatcgatga

SEQ ID NO: 46 - DNA, 22 bp, Artificial Sequence
  Synthetic oligonucleotide primer.
atggaatgcttgaaggttgccc

SEQ ID NO: 47 - DNA, 20 bp, Artificial Sequence
  Synthetic oligonucleotide primer.
actcccttgctcttcccgat

SEQ ID NO: 48 - DNA, 24 bp, Artificial Sequence
  Synthetic oligonucleotide primer.
tgcctttgaggcattcatgttggg

SEQ ID NO: 49 - DNA, 24 bp, Artificial Sequence
  Synthetic oligonucleotide primer.
atcacaggcaaggaaagggaggat

SEQ ID NO: 50 - DNA, 24 bp, Artificial Sequence
  Synthetic oligonucleotide primer.
ttccaaccacacccacagagtctt

SEQ ID NO: 51 - DNA, 24 bp, Artificial Sequence
  Synthetic oligonucleotide primer.
tctgggttgccatcgaacttctct

SEQ ID NO: 52 - DNA, 24 bp, Artificial Sequence
  Synthetic oligonucleotide primer.
tctcttccgcatgggatttgaggt

SEQ ID NO: 53 - DNA, 24 bp, Artificial Sequence
  Synthetic oligonucleotide primer.
tccagggtttccttcttggtgact

SEQ ID NO: 54 - DNA, 24 bp, Artificial Sequence
  Synthetic oligonucleotide primer.
aggtgcgcctattgtaggcagtta

SEQ ID NO: 55 - DNA, 24 bp, Artificial Sequence
  Synthetic oligonucleotide primer.
tcgccatcacaggaagggaaagat

SEQ ID NO: 56 - DNA, 24 bp, Artificial Sequence
  Synthetic oligonucleotide primer.
gcaaagatgatccctaggtgtgct

SEQ ID NO: 57 - DNA, 20 bp, Artificial Sequence
  Synthetic oligonucleotide primer.
gcaggagcagtctaccccaa

SEQ ID NO: 58 - DNA, 24 bp, Artificial Sequence
  Synthetic oligonucleotide primer.
cacccaggaccttccactcattta

SEQ ID NO: 59 - DNA, 24 bp, Artificial Sequence
  Synthetic oligonucleotide primer.
gaagctaagctgcagagcctcaaa

SEQ ID NO: 60 - DNA, 24 bp, Artificial Sequence
  Synthetic oligonucleotide primer.
cgctgattaggctccaaccatact

SEQ ID NO: 61 - DNA, 24 bp, Artificial Sequence
  Synthetic oligonucleotide primer.
attccttcagcactctggttcctc

SEQ ID NO: 62 - DNA, 20 bp, Artificial Sequence
  Synthetic oligonucleotide primer.
gcctgcagcaggaggatctt

SEQ ID NO: 63 - DNA, 24 bp, Artificial Sequence
  Synthetic oligonucleotide primer.
aagcccttcatccttcctcttagc

SEQ ID NO: 64 - DNA, 20 bp, Artificial Sequence
  Synthetic oligonucleotide primer.
tcggcgccggggagatacat

SEQ ID NO: 65 - DNA, 24 bp, Artificial Sequence
  Synthetic oligonucleotide primer.
gtctctctcttcctcttgtgctct

SEQ ID NO: 66 - DNA, 22 bp, Artificial Sequence
  Synthetic oligonucleotide primer.
gcaaagtactggatgacacgct

SEQ ID NO: 67 - DNA, 22 bp, Artificial Sequence
  Synthetic oligonucleotide primer.
ccctcagcttgctttttaggag

SEQ ID NO: 68 - DNA, 19 bp, Artificial Sequence
  Synthetic oligonucleotide primer.
ggatccatgcagcgtggac

SEQ ID NO: 69 - DNA, 20 bp, Artificial Sequence
  Synthetic oligonucleotide primer.
aaggtgaagtgtcccagagg

SEQ ID NO: 70 - DNA, 25 bp, Artificial Sequence
  Synthetic oligonucleotide primer.
gaggcaccaagttgggcatgaacga

SEQ ID NO: 71 - DNA, 23 bp, Artificial Sequence
  Synthetic oligonucleotide primer.
agtacaacaccatgctgggctat

SEQ ID NO: 72 - DNA, 19 bp, Artificial Sequence
  Synthetic oligonucleotide primer.
acccaaaacctggcgagat

SEQ ID NO: 73 - DNA, 21 bp, Artificial Sequence
  Synthetic oligonucleotide primer.
aagcaaccagagcgtgaagaa

SEQ ID NO: 74 - DNA, 19 bp, Artificial Sequence
  Synthetic oligonucleotide primer.
cctccccgactctgtttcc

SEQ ID NO: 75 - DNA, 17 bp, Artificial Sequence
  Synthetic oligonucleotide primer.
gtcggcccagccagagt

SEQ ID NO: 76 - DNA, 22 bp, Artificial Sequence
  Synthetic oligonucleotide primer.
tgtctccgaggactagccaagt

SEQ ID NO: 77 - DNA, 25 bp, Artificial Sequence
  Synthetic oligonucleotide primer.
gaaggagaacaaggagttgatgaag

SEQ ID NO: 78 - DNA, 20 bp, Artificial Sequence
  Synthetic oligonucleotide primer.
cccttcgagagcaagtggaa

SEQ ID NO: 79 - DNA, 20 bp, Artificial Sequence
  Synthetic oligonucleotide primer.
ggtggtctcctccgacttca

SEQ ID NO: 80 - DNA, 17 bp, Artificial Sequence
  Synthetic oligonucleotide primer.
cccactggtgccgtgaa

SEQ ID NO: 81 - DNA, 15 bp, Artificial Sequence
  Synthetic oligonucleotide primer.
tgctggccgcatgga

SEQ ID NO: 82 - DNA, 20 bp, Artificial Sequence
  Synthetic oligonucleotide primer.
tccaggtcggtctgggtaac

SEQ ID NO: 83 - DNA, 17 bp, Artificial Sequence
  Synthetic oligonucleotide primer.
tccccacgcagcaacac

SEQ ID NO: 84 - DNA, 21 bp, Artificial Sequence
  Synthetic oligonucleotide primer.
cacaactccaaccagtgcaaa

SEQ ID NO: 85 - DNA, 21 bp, Artificial Sequence
  Synthetic oligonucleotide primer.
cggctaccatcatctccattg

SEQ ID NO: 86 - DNA, 20 bp, Artificial Sequence
  Synthetic oligonucleotide primer.
gccctggtgaggatcagaaa

SEQ ID NO: 87 - DNA, 21 bp, Artificial Sequence
  Synthetic oligonucleotide primer.
cctccacaaccagctgaaaaa

SEQ ID NO: 88 - DNA, 20 bp, Artificial Sequence
  Synthetic oligonucleotide primer.
gcggagctcgatgtcatcat

SEQ ID NO: 89 - DNA, 23 bp, Artificial Sequence
  Synthetic oligonucleotide primer.
accaggaaatgagcttgacaaag

SEQ ID NO: 90 - DNA, 21 bp, Artificial Sequence
  Synthetic oligonucleotide probe.
ttggcaaattgctcgagttct

SEQ ID NO: 91 - DNA, 14 bp, Artificial Sequence
  Synthetic oligonucleotide probe.
agaggcacctggcc

SEQ ID NO: 92 - DNA, 20 bp, Artificial Sequence
  Synthetic oligonucleotide probe.
agtaatgatcccataacatt

SEQ ID NO: 93 - DNA, 16 bp, Artificial Sequence
  Synthetic oligonucleotide probe.
agcgggttttgggtct

SEQ ID NO: 94 - DNA, 16 bp, Artificial Sequence
  Synthetic oligonucleotide probe.
ccgtcccttctgaatt

SEQ ID NO: 95 - DNA, 16 bp, Artificial Sequence
  Synthetic oligonucleotide probe.
aggaaggagtttggcc

SEQ ID NO: 96 - DNA, 15 bp, Artificial Sequence
  Synthetic oligonucleotide probe.
tggaggcagatgaat

SEQ ID NO: 97 - DNA, 16 bp, Artificial Sequence
  Synthetic oligonucleotide probe.
aggtgtttccaaagaa

SEQ ID NO: 98 - DNA, 15 bp, Artificial Sequence
  Synthetic oligonucleotide probe.
ccacccctgaggatg

SEQ ID NO: 99 - DNA, 24 bp, Artificial Sequence
  Synthetic oligonucleotide probe.
cccactcttccaccttcgacgctg

SEQ ID NO: 100 - DNA, 24 bp, Artificial Sequence
  Synthetic oligonucleotide probe.
ggactcctccgggttttgctccag

SEQ ID NO: 101 - DNA, 24 bp, Artificial Sequence
  Synthetic oligonucleotide primer.
agtgcaggctcacacatcatagtc

SEQ ID NO: 102 - DNA, 24 bp, Artificial Sequence
  Synthetic oligonucleotide primer.
tagtctctgagcaagtagcgcatc

(19) United States
(12) Patent Application Publication
(10) Pub. No.: US 2010/0144549 A1
(43) Pub. Date: Jun. 10, 2010

(54) PARTHENOTE-DERIVED STEM CELLS AND METHODS OF MAKING AND USING THEM

(76) Inventor: Shoukhart M. Mitalipov, Hillsboro, OR (US)

Correspondence Address: KLARQUIST SPARKMAN, LLP, 121 SW SALMON STREET, SUITE 1600, PORTLAND, OR, 97204, US

(21) Appl. No.: 12/452,750

(22) PCT Filed: Jul. 18, 2008
(86) PCT No.: PCT/US08/70533

§ 371 Date: Jan. 19, 2010

Related U.S. Application Data
(60) Provisional application No. 60/961,385, filed on Jul. 20, 2007.

(51) Int. Cl. C40B 40/02; C12N 5/071; C12N 5/075

(52) U.S. Cl. 506/14; 435/363; 435/366

(57) ABSTRACT

Primate parthenotes, cells derived from them, and libraries of such cells are disclosed. Additionally, methods are disclosed for making primate parthenotes, the production of embryonic cells from these parthenotes, and for differentiating the partenotes into desired cell types, including multi-potent and differentiated cells. Methods are also provided for treating diseases or conditions for which the desired cell types are beneficial. Methods to identify agents of interest using these cells are also described.

### CROSS-REFERENCE TO RELATED APPLICATIONS

This claims the benefit of U.S. Provisional Application No. 60/961,385, filed Jul. 20, 2007, which is incorporated herein by reference in its entirety.

### STATEMENT REGARDING FEDERALLY SPONSORED RESEARCH OR DEVELOPMENT

This invention was made with United States government support pursuant to Grant Nos. NSO44330, HD18185, and RR00163 from the National Institutes of Health; the U.S. government has certain rights in the invention.

### BACKGROUND OF THE INVENTION

Embryonic stem cells (ESCs) are pluripotent cells derived from the inner cell mass (ICM) of a blastocyst that carry the potential to differentiate into lineages representing the three major germ layers. ESCs and their differentiated derivatives have the potential to replace defective cells by cell or tissue replacement therapy. At present, the risks of ESC-derived tissue transplantation in humans are high primarily because of safety issues, such as the potential for spontaneous or uncontrolled cellular proliferation. Therefore, therapeutic studies of ESCs in clinically relevant animal models such as nonhuman primates would be beneficial. Old World macaques serve as relevant models for which ESCs are available and have recently been used to generate dopaminergic neurons that function in animals with induced Parkinson symptoms.

Human ESCs derived from spare IVF embryos are genetically divergent from the host/patient. Thus, the transplantation of cells derived from such embryos (without immunosuppressive drugs) will likely incite an immune response resulting in rejection. Therapeutic cloning using somatic cell nuclear transfer to produce ESCs genetically identical to the patient may avoid immune rejection but has not yet been realized in humans. The anticipated high cost and current ethical issues regarding using IVF embryos provide compelling reasons to explore alternative sources of histocompatible, pluripotent cells. The generation of ESCs from parthenogenetic embryos can provide an alternative source of immunologically matched cells for transplantation therapy. Diploid parthenotes have been described. For example, diploid parthenotes were reportedly capable of completing preimplantation development and implanting, but were unable to form viable fetuses. The use of a human parthenote that cannot develop into a viable fetus may bypass some ethical concerns fundamental to the derivation of ESCs since it does not necessitate the destruction of potential human life.

Due to the limitations of current cell-based therapies, there remains a significant interest in and need for additional or alternative cell-based therapies. In particular, parthenogenetic embryonic stem cells (PESCs) that are genetically and epigenetically variable are desired. Transplantable cells derived from PESCs that can be transplanted without causing cancer or other adverse side-effects are also needed. Diploid parthenotes can be used to produce a variety of cell types, including both pluripotent and differentiated cells. These cells can be used for drug screening, various biological assays, and can also be used for therapeutic purposes.

### BRIEF SUMMARY OF THE INVENTION

Libraries are disclosed herein that include any of the primate parthenotes described herein and/or cells derived from any of the primate parthenotes described herein. In some embodiments, the library includes isolated primate parthenotes or isolated cells derived from a primate parthenote. In some embodiments, the parthenote or cell includes a set of heterozygous MEW alleles and a set of heterozygous alleles for a gene that does not encode an MEW molecule. In some embodiments, a second isolated parthenote or isolated cell derived from an isolated primate parthenote is included in the library. This parthenote or cell includes a set of heterozygous MEW alleles and a set of homozygous alleles for a gene that does not encode an MEW molecule. In some embodiments, an additional isolated parthenote or isolated cell is included in the library. This parthenote or cell derived from an isolated primate parthenote includes a set of homozygous WIC alleles and a set of homozygous alleles for a gene that does not encode an WIC molecule. In some embodiments, the library also includes yet another isolated or purified primate parthenote or cell derived from an isolated or purified primate parthenote. This parthenote or cell includes a set of homozygous MHC alleles and a set of heterozygous alleles for a gene that does not encode an WIC molecule.

In additional embodiments, methods are disclosed for making primate parthenotes, and for producing cells from these primate parthenotes.

Cells produced form these primate parthenotes are also disclosed herein. In several examples, the cells are pluripotent embryonic stem cells or differentiated endodermal, mesodermal or ectodermal cells. Pharmaceutical compositions and kits including these cells are also described herein.

Other embodiments of the invention will be apparent from the following detailed description and from the claims.

### BRIEF DESCRIPTION OF THE DRAWINGS

FIG. 1 is a scheme illustrating an exemplary mechanism for the formation of diploid heterozygous parthenotes.

FIGS. 2A-E illustrate the determination of pluripotency and imprinted gene expression in monkey PESCs. FIG. 2A illustrates the expression of pluripotent markers detected by immunocytochemistry. Panels A and C in FIG. 2A are phase contrast micrographs of PESC colonies growing on feeder layers. Panels B and D in FIG. 2A are the same colonies as in panels A and C fixed and immunolabeled with primary antibodies against OCT4 and SSEA-4, respectively, and secondary antibodies conjugated with Cy3. Only PESC colonies were positive for stem cell markers, while feeder cells were negative. FIG. 2B illustrates the RT-PCR detection of stemness markers. FIGS. 2C and 2D illustrate the expression panel of known maternally (FIG. 2C) and paternally expressed imprinted genes (FIG. 2D) (see, for example, the world wide web at “geneimprint.com/site/home”) in monkey PESCs detected by RT-PCR. Bi-parental ORMSE-22 served as a control. FIG. 2E illustrates the quantitative real-time PCR analyses of selected paternally and maternally expressed genes in parthenogenetic (R1−RPESC-1) and bi-parental (OR-22−ORMES-22) monkey ESCs. The mean expression level was calculated using standard curve method followed by normalization with housekeeping GAPDH. Data represent the means±S.E.M. (n=3).

FIGS. 3A and 3B illustrate the methylation analysis of the IGF2/H19 imprinting center in monkey PESCs by Southern-blot analysis and bisulfite sequencing. FIG. 3A shows gDNA isolated from monkey parthenogenetic (RPESC-1, -2, and -3), bi-parental ESCs (ORMES-22), and muscle tissue that was cut with EcoNI and methylation specific BsaHI. The larger (upper) band corresponds to a 1.3 kb uncut methylated fragment, and the smaller (lower) band corresponds to the 1.07 kb digested unmethylated fragment. The two EcoNI restriction sites are located 17 by upstream and 294 by downstream of the H19CTCF-6 gDNA sequence (GenBank Acc# AY725988). The CpG methylation-blocked BsaHI restriction site is located at the 205 by nucleotide position corresponding to GenBank Acc# AY725988. Occurrence of both bands represents the presence of both methylated and unmethylated alleles in the same sample. FIG. 3B shows genomic DNA that was modified by bisulfite treatment, followed by PCR amplification. PCR products were cloned, and a minimum of 10 randomly selected clones were sequenced for each sample. Each circle represents an individual CpG site; methylated CpG dinucleotides are depicted by black circles, and the unmethylated sites are depicted as open circles. The boxed area corresponds to the CTCF-6 core binding site. Note that sperm DNA was completely methylated, and control muscle maintained unmethylated maternal (Mat) and methylated paternal (Pat) alleles (separated by the presence of SNP). In contrast, bi-parental ORMES-22 and PRESC lines were hypermethylated as a result of sporadic methylation of maternal alleles.

FIG. 4 illustrates the cytogenetic analysis of rhesus monkey PESCs by G-banding demonstrating normal euploid karyotypes (42, XX) for RPESC-2, -3, -4, and -5 that were indistinguishable from fertilized female rhesus karyotypes. G-banding analysis of RPESC-1 revealed that the majority of analyzed cells (24/32) displayed cytogenetically normal XX female chromosome complement. However, remaining cells exhibited unbalanced chromosomal abnormalities: two of 32 analyzed cells were characterized by the presence of three copies of chromosome 18, and six cells were characterized by the presence of three copies of chromosomes 14 and 18. It is unclear at this point whether these abnormalities were inherited from the parental embryo or occurred during ESC culture.

FIGS. 5A-5I illustrate the analysis of the differentiation potential of PESC lines in teratomas. FIG. 5A illustrates the cystic areas within a teratoma (×40). FIG. 5B illustrates the ctoderm-derived stratified squamous epithelium with some areas of keratinization (×100). FIG. 5C illustrates the mesoderm-derived cartilage and bone (×100). FIG. 5D illustrates the endoderm-derived intestinal-type columnar epithelium (×100). FIG. 5E illustrates the immature neuroectodermal tissue (×400). FIGS. 5F-I illustrate muscle tissue (×100 and ×400).

FIG. 6 illustrates the methylation status of the SNURF/SNURPN IC involved in the regulation of paternal imprints in the PWS region in monkey PESCs, bi-parental ORMES-22, muscle, and sperm. Each circle represents an individual CpG dinucleotide; methylated CpG sites are depicted by black circles, and the unmethylated sites are depicted as open circles. Oocyte-derived PESC lines were heavily methylated, while all sequenced clones of mature sperm were completely unmethylated. In bi-parental ORMES-22 and muscle DNA, both unmethylated and methylated clones were observed.

FIG. 7 is a set of bar graphs showing the expression of paternally imprinted genes in ORMES-22 (biparental), ORMES-9 (homozygous parthenote) and rPESC-2 (heterozygous parthenote) determined by microarray and q-PCR. In the bar graphs on the right, the validation of microarray results by q-PCR is displayed for 11 genes. The X axis represents analyzed cell lines (a: ORMES-22, b: ORMES-9 and c: rPESC-2). Microarray results are in the lighter columns on the left y-axis, and q-PCR results are in darker columns on the right y-axis shows the relative expression levels of each imprinted gene as determined by comparison to the expression level of housekeeping control GAPDH (imprinted gene:GAPDH ratio). The mean expression level was calculated using a standard curve method followed by normalization with housekeeping GAPDH. Data represent the means±S.E.M. (n=6).

FIG. 8 is a set of bar graphs showing the expression of maternally imprinted genes in ORMES-22 (biparental), ORMES-9 (homozygous parthenote) and rPESC-2 (heterozygous parthenote) detected by microarray and quantitative (q) PCR. The bar graphs on the right show the validation of the microarray results by Q-PCR, displayed for 9 genes. The X axis represents analyzed cell lines (a: ORMES-22, b: ORMES-9 and c: rPESC-2). Microarray results are in lighter columns on the left y-axis, and q-PCR results are in darker columns on the right y-axis shows the relative expression levels of each imprinted gene as determined by comparison to the expression level of housekeeping control GAPDH (imprinted gene:GAPDH ratio). The mean expression level was calculated using a standard curve method followed by normalization with housekeeping GAPDH. Data represent the means±S.E.M. (n=6).

FIG. 9 is a bar graph showing relative telomere length. The x-axis represents the cell lines analyzed, ORMES-22 (in vitro fertilization (IVF)-derived biparental), ORMES-9 (homozygous parthenote) and rPESC-2 (heterozygous parthenote). The y-axis shows the relative telomere length (telomere/single copy gene ratio). The data represents the mean±SEM (n=4). Both heterozygous and homozygous parthenogenetic ES cell lines shown significant elongation of telomere length similar to bi-parental ES cells as compared to donor fibroblasts.

FIG. 10 is a bar graph showing the X-inactivation status in parthenogenetic ESCs. Quantitative RT-PCR demonstrates expression of XIST in female fibroblast and undifferentiated female ESCs, ORMES-22 (IVF-derived biparental), ORMES-9 (homozygous parthenote) and rPESC-2 (heterozygous parthenote). X-axis represents the cell lines analyzed. The y-axis shows the relative XIST expression (XIST/GAPDH ratio). The data represents the mean±SEM (n=4). Expression of XIST is not observed in male fibroblasts.

### DETAILED DESCRIPTION OF SEVERAL EMBODIMENTS

Diploid primate parthenogenetic cells are described herein, as well as totipotent or pluripotent stem cells or transplantable cells derived from them as well as pharmaceutical compositions, kits, and methods that include these cells. Strikingly, these cells are heterozygous and express many imprinted genes that are normally expressed from the paternal alleles. These characteristics make the cells desirable for a variety of cell and tissue replacement therapies and for research and drug screening applications (e.g., applications related to the study or modulation of cell division, chromosome behavior, homologous recombination, genomic imprinting, self-renewal, differentiation, maturation, or migration). For example, these cells are able to differentiate into all the cell types that have been tested, supporting their use in a therapeutic context for autologous or immuno-matched transplantations. The isolated parthenotes or cells are primate parthenotes or cells, such as non-human primate or human partenotes or cells.

Because these parthenotes can be generated at efficiencies comparable to those of sperm-fertilized controls, the parthenotes or cells derived from them can be used to generate cell banks for cell transplantation applications. The cell banks can provide cells with WIC allele matches for a large percentage of the potential recipient pool, allowing the cell transplantation methods described herein to be used for a large number of recipients. While not intending to be bound by any particular theory, the high efficiencies observed for the generation of primate parthenotes may be due in part to the high quality oocytes obtained from the oocyte harvesting methods described herein and/or to the high efficiency of the methods for inducing parthenogenesis described herein.

Libraries are disclosed herein that include any of the primate parthenotes described herein and/or cells derived from any of the primate parthenotes described herein. In some embodiments, the library contains isolated or purified primate parthenotes or cells derived from an isolated primate parthenote. In some embodiments, the parthenote or cell includes a set of heterozygous WIC alleles and a set of heterozygous alleles for a gene that does not encode an WIC molecule. In some embodiments, a second parthenote or cell derived from an isolated or purified primate parthenote is included in the library. This parthenote or cell includes a set of heterozygous WIC alleles and a set of homozygous alleles for a gene that does not encode an WIC molecule. In some embodiments, an additional parthenote or cell is included in the library. This parthenote or cell derived from an isolated or purified primate parthenote includes a set of homozygous WIC alleles and a set of homozygous alleles for a gene that does not encode an MEW molecule. In some embodiments, the library also includes yet another isolated or purified primate parthenote or cell derived from an isolated or purified primate parthenote. This parthenote or cell includes a set of homozygous MHC alleles and a set of heterozygous alleles for a gene that does not encode an MEW molecule.

In some embodiments, the library includes about any of 5, 10, 15, 20, 50, 100, 200, 500, 1,000, or more isolated or purified primate parthenotes or cells derived from an isolated or purified primate parthenote. In some embodiments, the first parthenote or cell and/or the second parthenote or cell is heterozygous for HLA-A, HLA-B, HLA-DR, or any two or more of the foregoing alleles. In some embodiments, the first parthenote or cell and/or the second parthenote or cell is heterozygous for all of the HLA-A, HLA-B, and HLA-DR alleles. In some embodiments, the first parthenote or cell and/or the second parthenote or cell is heterozygous for one or more of the following MEW-linked microsatellite loci: D6S291, D6S2741, D6S2876, 9P06, DRA, MICA, 246K06, 162B17A, 162B17B, 151L13, MOGCA, 268P23, 222I18, D6S276, and D6S1691. In some embodiments, at least one parthenote or cell expresses a paternally expressed gene.

In additional embodiments, an isolated or purified primate cell is disclosed that is a parthenote or a cell (such as a multipotent stem cell or transplantable cell) derived from a parthenote, where the parthenote is heterozygous at one or more genes. In some embodiments, the cell has two different alleles for at least about any of 5, 10, 20, 50, 100, 1000, or more genes. In some embodiments, the cell has two different alleles for at least about any of 5%, 10%, 25%, 50%, 60%, 70%, or more of all the genes from the species of the individual from which the oocyte used to generate the cell was derived. In various embodiments, the cell is heterozygous (or homozyogous) for a gene encoding an MEW molecule, such as an HLA-A, HLA-B, or HLA-DR molecule. In some embodiments, the cell is heterozygous (or homozygous) for one or more of the following MEW-linked microsatellite loci: D6S291, D6S2741, D6S2876, 9P06, DRA, MICA, 246K06, 162B17A, 162B17B, 151L13, MOGCA, 268P23, 222I18, D6S276, and D6S1691. In some embodiments, the cell has at least one set of homozygous MEW alleles (such as a homozygous set of alleles for HLA-A, HLA-B, or HLA-DR, or any two or more of the foregoing) and a heterozygous set of alleles for another gene (such as a gene that does not encode an MHC molecule). In particular embodiments, the cell has at least one set of homozygous MHC alleles for one or more of the following WIC-linked microsatellite loci: D6S291, D6S2741, D6S2876, 9P06, DRA, MICA, 246K06, 162B17A, 162B17B, 151L13, MOGCA, 268P23, 222I18, D6S276, and D6S1691. In various embodiments, the cell expresses at least about any of 2, 5, 10, 20, 40, 60, 80, 100, 200, or more parentally imprinted genes. In additional embodiments, the cell has telomeres about the length of the telomeres in a pluripotent embryonic stem cell.

An isolated primate cell is disclosed herein that is a parthenote or a cell (such as a multipotent stem cell or transplantable cell) derived from a parthenote. The primate parthenote or cell is a non-human primate parthenote or cell or a human parthenote or cell. In some embodiments, the parthenote or cell expresses a paternally expressed gene. In some embodiments, the parthenote or cell expresses at least about any of 2, 5, 10, 20, 50, 100, 1000, or more parentally imprinted genes. In some embodiments, the cell expresses at least about any of 5%, 10%, 25%, 50%, 60%, 70%, or more of all the parentally imprinted genes from the species of the individual from which the oocyte used to generate the cell was derived. In some embodiments, the parthenote or cell expresses one or more of OCT4, SSEA-3, SSEA-4, TRA-1-60, TRA-1-81, NANOG, SOX-3, TDGF, THY1, FGF4, LEFTYA, and TERT. In various embodiments, the parthenote or cell contains a methylated H19/IGF2 imprinting center. In additional embodiments, the parthenote or cell has telomeres about the length of the telomeres in a pluripotent embryonic stem cell

In some embodiments, the cell is pluripotent. In some embodiments, the cell is a monkey cell, such as a rhesus monkey cell, or a human cell. In some embodiments, the cell derived from a parthenote is an ectodermal cell, such as a stratified squamous epithelial, neural, or ganglion cell. In some embodiments, the cell derived from a parthenote is a mesodermal cell, such as a cardiomyocyte or a cartilage, hepatocyte, smooth muscle cell, bone, muscle, fibrous connective tissue, or blood cell. In some embodiments, the cell derived from a parthenote is an endodermal cell, such as an enteric-type columnar epithelial cell. In various embodiments, the cell derived from a parthenote is a non-immortalized cell. In some embodiments, the cell derived from a parthenote is a trophoblast cell. In some embodiments, the cell derived from a parthenote is a totipotent stem cell.

In additional embodiments, an in vitro method is provided for producing an isolated primate parthenote (such as any of the parthenotes described herein). The method can include harvesting an oocyte (such as a MI or MII oocyte) and activating the oocyte via incubation in vitro with ionomycin under conditions sufficient to generate a primate parthenote that is heterozygous and/or expresses a paternally expressed gene. In some embodiments when primates are stimulated to produce oocytes (e.g., hormonally) and these oocytes are harvested, the oocytes that are collected can be in different phases. Some oocytes are in metaphase I while other oocytes are in metaphase II. In such cases, the oocytes that are in metaphase I can be put into culture until they reach metaphase II and then used for parthenogenetic activation. Optionally, the oocytes that have been cultured to reach metaphase II are combined with the oocytes that were already at metaphase II when harvested for a pool of potential oocytes. In some embodiments, only the oocytes that are in metaphase II from the harvest are used for parthenogenetic activation.

In some embodiments, the oocyte is activated by incubation in vitro with ionomycin under conditions sufficient to generate a primate parthenote that is heterozygous and/or expresses a paternally expressed gene. In various embodiments, the concentration of ionomycin is between about 1 μM to about 20 μM, such as between about 1 μM to about 10 μM, about 3 μM to about 7 μM, or about 5 μM to about 10 μM, or about 5 μM. In other examples, the concentration of ionomycin is about 10 μM to about 15 μM, or about 15 μM to about 20 μM. In one example, the concentration of ionomycin is about 5 μM. In some embodiments, the concentration of ionomycin is about any of 1, 2, 3, 4, 5, 6, 7, 8, 9, or 10 μM. In various embodiments, the oocyte is incubated with ionomycin for between about 1 to about 20 minutes, such as between about 1 to about 10 minutes, such as about 3 to about 7 minutes, or about 4 or about 5 minutes. However, the oocyte can be incubated with ionomycin for about 10 to about 15 minutes, or about 15 to about 20 minutes. In some embodiments, the oocyte is incubated with ionomycin for at least about any of 1, 2, 3, 4, 5, 6, 7, 8, 9, or 10 minutes. In some embodiments, the oocyte is incubated with ionomycin for about any of 1, 2, 3, 4, 5, 6, 7, 8, 9, or 10 minutes. In some embodiments, the oocyte is incubated in a medium (such as TALP/HEPES medium) with ionomycin and a first concentration of serum albumin (e.g., bovine or human serum albumin) as a first incubation.

In some embodiments, the method also includes incubating the oocyte in a medium (such as TALP/HEPES medium) with a second concentration of serum albumin (e.g., bovine or human serum albumin) that is greater than the first concentration of serum albumin (e.g., bovine or human serum albumin) as a second incubation. In some embodiments, the duration of the second incubation is between about 1 to about 20 minutes, such as between about 1 to about 10 minutes, about 3 to about 7 minutes, or about 5 minutes. However, in other examples, the oocyte is incubated for about 5 to about 10 minutes, about 10 to about 15 minutes, or about 15 to about 20 minutes. In some embodiments, the duration of the second incubation is about any of 1, 2, 3, 4, 5, 6, 7, 8, 9, or 10 minutes. In particular embodiments, the first incubation is about 5 minutes in duration, and the second incubation is about 5 minutes in duration. In various embodiments, the first concentration of serum albumin (e.g., bovine or human serum albumin) is between about 0.1 mg/mL to about 10 mg/mL, such as about 0.5 to about 5 mg/mL, or about 2 mg/mL. In additional embodiments, the first concentration of serum albumin is between about 0.1 mg/mL to about 0.5 mg/mL, about 0.5 mg/mL to about 1 mg/mL, about 1 mg/mL to about 2.5 mg/mL, about 2.5 mg/mL to about 5 mg/mL, about 5 mg/mL to about 7.5 mg/mL, or about 7.5 mg/mL to about 10 mg/mL. In particular embodiments, the first concentration of serum albumin (e.g., bovine or human serum albumin) is about any of 0.1, 0.5, 1, 2, 3, 4, 5, 6, 7, 8, 9, or 10 mg/mL. In various embodiments, the second concentration of serum albumin (e.g., bovine or human serum albumin) is about 10 mg/mL to about 60 mg/ml, such as about 20 to about 40 mg/mL, or about 30 mg/mL. In other embodiments, the second concentration of serum albumin is between about 10 mg/mL to about 20 mg/mL, about 20 mg/mL to about 30 mg/mL, about 30 mg/mL to about 40 mg/mL, about 40 mg/mL to about 50 mg/mL, or about 50 mg/mL to about 60 mg/mL. In particular embodiments, the second concentration of serum albumin (e.g., bovine or human serum albumin) is about any of 10, 20, 25, 30, 35, 40, 50, or 60 mg/mL. In some embodiments, the ratio of the first concentration of serum albumin to the second concentration of serum albumin is about any of 1:2, 1:3, 1:4, 1:5, 1:6, 1:10, 1:15, 1:20, 1:25, 1:30, 1:35, 1:40, 1:50, or 1:60. In some embodiments, the medium comprises 6-dimethylaminopurine. In some embodiments, the concentration of 6-dimethylaminopurine is between about 0.1 to about 10 mM, such as between about 0.5 μm to about 5 μM, such as about 1 μM to about 3 μM, or about 2 μM. In other examples, the concentration of 6-methylaminopurine is 0.1 mM to about 0.5 mM, about 0.5 mM to about 1 mM, about 1 mM to about 2.5 mM, about 2.5 mM to about 5 mM, about 5 mM to about 7.5 mM, or about 7.5 mM to about 10 mM. In some embodiments, the medium comprises as at least about 2 mM or comprises about 2 mM 6-dimethylaminopurine.

In some embodiments, the method further includes incubating the oocyte in a medium (such as HECM-9 medium) as a third incubation. In some embodiments, the duration of the third incubation is between about 1 to about 10 hours, such as about 2 to about 8 hours, such as about 3 to about 6 hours, or about 5 hours. In other examples, the third incubation is between about 1 to about 2.5 hours, about 2.5 to about 5 hours, about 5 to about 7.5 hours, or about 7.5 to about 10 hours. In particular embodiments, the duration of the third incubation is about any of 1, 2, 3, 4, 5, 6, 7, 8, 9, or 10 hours. In various embodiments, the third incubation is greater than 4 hours in duration, such as about any of 5, 6, 7, 8, 9, or 10 hours in duration. In some embodiments, the medium comprises 6-dimethylaminopurine. In some embodiments, the concentration of 6-dimethylaminopurine is about 0.1 to about 10 mM, such as between about 0.5 μm to about 5 μM, such as about 1 μM to about 3 μM, or about 2 μM. In additional embodiments, the concentration of 6-dimethylaminopurine is between about 0.1 to about 10 mM, such as between about 0.1 mM to about 0.5 mM, about 0.5 mM to about 1 mM, about 1 mM to about 2.5 mM, about 2.5 mM to about 5 mM, about 5 mM to about 7.5 mM, or about 7.5 mM to about 10 mM. In some embodiments, the medium comprises as at least about 2 mM or comprises about 2 mM 6-dimethylaminopurine. In some embodiments, the method also includes incubating the oocyte in a medium (such as HECM-9 medium) as a second incubation. In various embodiments, the medium does not include 6-demethylaminopurine but comprises cytochalasin B and either roscovitine or cycloheximide. In some embodiments, the concentration of cytochalasin B is between about 0.5 μg/mL to about 20 μg/mL, such as between about 1 μg/mL to about 20 μg/mL, 2 μg/mL to about 10 μg/mL, or between about 4 μg/ml to about 6 μg/ml, or about 5 μg/mL cytochalsin B. In additional embodiments, the concentration of cytochalasin B is 0.5 μg/mL to about 1 μg/mL, about 1 μg/mL to about 2.5 μg/mL, about 2.5 μg/mL to about 5 μg/mL, about 5 μg/mL to about 7.5 μg/mL, about 7.5 μg/mL to about 10 μg/mL, about 10 μg/mL to about 12.5 μg/mL, about 12.5 μg/mL to about 15 μg/mL, or about 15 μg/mL to about 20 μg/mL. In some embodiments, the concentration of cytochalasin B is about any of 1, 2, 3, 4, 5, 6, 7, 8, 9, or 10 μg/mL. In particular embodiments, the concentration of cytochalasin B is about 5 μg/mL. In some embodiments, the concentration of roscovitine is between about 5 μM to about 200 μM, such as between about 5 μM to about 100 μM, 25 μM to about 75 μM, or about 50 μM roscotivine. In other embodiments, the concentration of roscovitine is about 5 μM to about 10 μM, about 10 μM to about 25 μM, about 25 μM to about 50 μM, about 50 μM to about 75 μM, about 75 μM to about 100 μM, about 100 μM to about 125 μM, about 125 μM to about 150 μM, or about 150 μM to about 200 μM. In some embodiments, the concentration of roscovitine is about any of 10, 20, 30, 40, 50, 60, 70, 80, 90, or 100 μM. In particular embodiments, the concentration of roscovitine is about 50 μM. In some embodiments, the concentration of cycloheximide is between about 0.5 μg/mL to about 20 μg/mL, 1 μg/mL to about 10 μg/mL, 5 μg/mL to about 8 μg/mL, or about 7.5 μg/mL. In additional embodiments, the concentration of cycloheximide between about 0.5 μg/mL to about 1 μg/mL, about 1 μg/mL to about 2.5 μg/mL, about 2.5 μg/mL to about 5 μg/mL, about 5 μg/mL to about 7.5 μg/mL, about 7.5 μg/mL to about 10 μg/mL, about 10 μg/mL to about 12.5 μg/mL, about 12.5 μg/mL to about 15 μg/mL, or about 15 μg/mL to about 20 μg/mL. In some embodiments, the concentration of cycloheximide is about any of 1, 2, 3, 4, 5, 6, 7, 8, 9, 10, 12, 14, 16, 18, or 20 μg/mL. In particular embodiments, the concentration of cycloheximide is about 7.5 μg/mL. In several embodiments, roscovitine and cytoclasin B are used together, or cycloheximide and cyotchalasin B are used together.

In some embodiments, the third incubation occurs between about 20 to about 45° C., such as at about 37° C. In some embodiments, the third incubation occurs in air with a CO₂ concentration of between about 1 to about 15%, such as about 1% to about 10%, so between about 3% to about 7%, such as at about 5%. In other examples, the concentration is between about 1 to about 5%, about 5 to about 10%, or about 10 to about 15% (e.g., concentrations based on volume). In some embodiments, the third incubation occurs in air with a CO₂ concentration of about any of 1, 2, 3, 4, 5, 6, 7, 8, 9, 10, 12, or 15%. In some embodiments, the third incubation occurs in air with an O₂ concentration of between about 1 to about 15%, such as about 1% to about 10%, or between about 3% to about 7%, such as at about 5%. In other examples, the concentration is between about 1 to about 5%, about 5 to about 10%, or about 10 to about 15%. In some embodiments, the third incubation occurs in air with an O₂ concentration of about any of 1, 2, 3, 4, 5, 6, 7, 8, 9, 10, 12, or 15%. In some embodiments, the third incubation occurs in air with an N₂ concentration of between about 70 to about 98%, such as between about 80% to about 98%, or between about 85% to about 95%, such as about 90%. In other examples, the concentration is 70 to about 80%, about 80 to about 90%, or about 90 to about 98%. In some embodiments, the third incubation occurs in air with a N₂ concentration of about any of 70, 75, 80, 85, 90, 95, or 98%. In some embodiments, the third incubation occurs in air with a CO₂ concentration of about 5%, an O₂ concentration of about 5%, and an N₂ concentration of about 90%.

In some embodiments, the duration of the second incubation is between about 1 to about 10 hours, such between about 1 to about 2.5 hours, about 2.5 to about 5 hours, about 5 to about 7.5 hours, or about 7.5 to about 10 hours. In particular embodiments, the duration of the second incubation is about any of 1, 2, 3, 4, 5, 6, 7, 8, 9, or 10 hours. In various embodiments, the second incubation is greater than 4 hours in duration, such as about any of 5, 6, 7, 8, 9, or 10 hours in duration.

In some embodiments, at least about any of 10%, 20%, 30%, 40%, 50%, 60%, 70%, 80%, 90%, 95%, or 100% of the parthenotes develop into cleaved embryos. In some embodiments, at least about any of 10%, 20%, 30%, 40%, 50%, 60%, 70%, 80%, 90%, 95%, or 100% of the cleaved embryos develop into an 8-cell embryo. In some embodiments, at least about any of 10%, 20%, 30%, 40%, 50%, 60%, 70%, 80%, 90%, 92%, 93%, 95%, or 100% of the cleaved embryos develop into a morula, such as a compact morula. In some embodiments, at least about any of 10%, 20%, 25%, 30%, 38%, 40%, 45%, 50%, 70%, or 80% of the cleaved embryos develop into a blastocyst.

In additional embodiments, methods are provided for producing a cell of a desired cell type using any of the primate parthenotes described herein or cells derived from them. The methods for producing a cell of a desired cell type can include incubating an isolated or purified primate parthenote that expresses a paternally expressed gene or a cell derived from such a primate parthenote under in vitro conditions sufficient to generate a cell of a desired cell type. In one such aspect, the invention features a method for deriving a cell of a desired cell type by incubating an isolated or purified heterozygous primate parthenote or a cell derived from a heterozygous parthenote under in vitro conditions sufficient to generate a differentiated cell or a stem cell. In various embodiments, the desired cell type is an ectodermal cell, such as a stratified squamous epithelial, neural, or ganglion cell. In some embodiments, the desired cell type is a mesodermal cell, such as a cardiomyocyte or a cartilage, bone, muscle, fibrous connective tissue, or blood cell. In some embodiments, the desired cell type is an endodermal cell, such as an enteric-type columnar epithelial cell. In some embodiments, the desired cell is a trophoblast cell. In some embodiments, the desired cell is a totipotent cell.

Additionally, methods are provided for treating a disease, disorder, or condition in an individual by administering an effective amount of one or more cells derived from any of the primate parthenotes described herein to an individual in need of one or more cell types. Methods are provided for treating a disease, disorder, or condition in an individual by administering an effective amount of one or more multipotent stem cells or transplantable cells derived from a heterozygous primate parthenote to an individual in need of one or more cell types. In some examples, the cell is a multipotent stem cell or transplantable cell derived from a primate parthenote that expresses a paternally expressed gene. In some embodiments, the cell differentiates into one or more of the cell types the individual is in need of following administration. In some embodiments, the cell is differentiated into one or more of the cell types the individual is in need of prior to administration to the individual. In some embodiments, the cell is a hematopoietic stem cell. For example, hematopoietic stem cells can be administered to treat cancer. In some embodiments, the oocyte used to generate the parthenote is from the individual or a relative of the individual. In various embodiments, the cell expresses more than one allele of a gene encoding an MHC molecule, such as an HLA-A, HLA-B, or HLA-DR molecule. In some embodiments, the cell expresses an HLA-A, HLA-B, or HLA-DR molecule that is identical to the corresponding protein in the individual. In some embodiments, the cell expresses a parentally imprinted gene, such as a cell expressing at least about any of 2, 5, 10, 20, 50, 100, 1000, or more parentally imprinted genes.

In additional embodiments, pharmaceutical composition are provided that include one or more multipotent stem cells or transplantable cells derived from any of the primate parthenotes described herein. In some embodiments, the pharmaceutical composition includes (i) a multipotent stem cell or transplantable cell derived from any of the primate parthenotes described herein and (ii) a pharmaceutically acceptable carrier. In various embodiments, the pharmaceutical composition includes (i) a multipotent stem cell or transplantable cell derived from a heterozygous primate parthenote and (ii) a pharmaceutically acceptable carrier. In further embodiments, the pharmaceutical composition includes (i) a multipotent stem cell or transplantable cell derived from a primate parthenote that expresses a paternally expressed gene and (ii) a pharmaceutically acceptable carrier.

Kits are also provided that include one or more multipotent stem cells or transplantable cells derived from any of the primate parthenotes described herein. In some embodiments, the kit includes (i) a multipotent stem cell or transplantable cell derived from any of the primate parthenotes described herein and (ii) instructions for using the kit to treat a disease, disorder, or condition in an individual. In some embodiments, the kit includes (i) a multipotent stem cell or transplantable cell derived from a heterozygous primate parthenote and (ii) instructions for using the kit to treat a disease, disorder, or condition in an individual. In some embodiments, the kit includes (i) a multipotent stem cell or transplantable cell derived from a primate parthenote that expresses a paternally expressed gene and (ii) instructions for using the kit to treat a disease, disorder, or condition in an individual.

Any of the parthenotes described herein or cells derived from them can be used to study cell division, chromosome behavior, recombination, genomic imprinting, self-renewal, differentiation, maturation, and/or migration. The parthenotes and cells described herein are also of use in screening assays, such as to identify whether a candidate compound modulates cell division, chromosome behavior, recombination, genomic imprinting, self-renewal, differentiation, maturation, or migration. Thus, methods are provided for determining whether a candidate compound modulates cell division, chromosome behavior, recombination, genomic imprinting, self-renewal, differentiation, maturation, or migration. In several examples, the method includes (a) contacting an isolated or purified heterozygous primate parthenote or a cell (such as a multipotent stem cell or transplantable cell) derived from an isolated or purified heterozygous primate parthenote with a candidate compound; and (b) assaying for cell division, chromosome behavior, recombination, genomic imprinting, self-renewal, differentiation, maturation, migration, or any two or more of the foregoing in the parthenote or the cell derived from the parthenote. The candidate compound is determined to modulate cell division, chromosome behavior, recombination, genomic imprinting, self-renewal, differentiation, maturation, or migration if the candidate compound causes a change in cell division, chromosome behavior, recombination, genomic imprinting, self-renewal, differentiation, maturation, or migration.

### Terms

Unless defined otherwise, all technical and scientific terms used herein have the same meaning as is commonly understood by one of ordinary skill in the art to which this invention belongs. If a definition set forth in this section is contrary to or otherwise inconsistent with a definition set forth in patents, published patent applications and other publications and sequences from GenBank and other databases that are herein incorporated by reference, the definition set forth in this section prevails over the definition that is incorporated herein by reference.

For use herein, unless clearly indicated otherwise, use of the terms “a”, “an,” and the like refers to one or more.

General reference to “the composition” or “compositions” includes and is applicable to compositions of the invention. The invention also provides pharmaceutical compositions comprising the components described herein.

Reference to “about” a value or parameter herein includes (and describes) embodiments that are directed to that value or parameter per se. For example, description referring to “about X” includes description of “X.”

As used herein, the term “embryo” refers generally to a cellular mass obtained by one or more divisions of a zygote or a parthenogenetically activated oocyte without regard to whether it has been implanted into a female.

By “allele” is meant one of the two or more alternative forms of a gene that can occur at a particular chromosomal site (locus), and which determine alternative characters in inheritance. Because autosomal chromosomes occur in pairs, an individual will have two alleles, which may be the same or different, for each specific locus. A “homzyogote” contains two copies of the same allele at a specific locus, while a “heterozygote” contains two different alleles at a specific locus. Similarly, “homozygous” refers to two copies of the same allele at a specific chromosomal locus in a cell or individual while “heterozygous” refers to two different alleles at a specific chromosomal locus in a cell or individual.

The term “DNA methylation” refers to the postsynthetic addition of methyl groups to specific sites on DNA molecules; the reaction is catalyzed by enzymes called DNA methyltransferases that are specific for nucleotide and position of methylation. In eukaryotes, methylation is involved in gene expression, and plays a role in a variety of epigenetic mechanisms, including development, X chromosome inactivation, genomic imprinting, mutability of DNA, and uncontrolled cell growth in cancer. The term “X chromosome inactivation” refers to the inactivation of one of each pair of X chromosomes to form the Barr body in female mammalian somatic cells. Thus tissues whose original zygote carried heterozygous X borne genes should have individual cells expressing one or other but not both of the X encoded gene products. The inactivation is thought to occur early in development and leads to mosaicism of expression of such genes in the body.

The phrase “dosage compensation” refers to a mechanism that senses gene dosage and regulates expression accordingly. In mammals there is monoallelic expression of X-linked genes that differ in dose between females (XX) and males (XY). “XIST” refers to a gene encoding a large non-coding RNA which has been shown to be necessary for developmentally regulated X chromosome silencing in females. The XIST RNA is about 18 kb and is not translated, it is spliced, and polyadenylated. It is also organized into blocks of repetitive sequence. In vivo, XIST RNA is found to be stably associated with the silenced X chromosome. The expression of XIST RNA is always cis-limited, and is associated with the silenced X chromosome in females.

“Genomic imprinting” refers to a mammalian epigenetic phenomenon whereby the parental origin of a gene determines whether or not it will be expressed. Over 75 imprinted genes have been identified, many of which are noncoding RNAs that are hypothesized to control the expression of linked protein coding genes that are also imprinted. Generally, allele-specific methylation of CpG dinucleotides is a mechanism that regulates gene expression of imprinted genes. “Maternally expressed” refers to a gene that is expressed from the copy inherited from the mother. Imprinted genes include, but are not limited to the maternally expressed imprinted genes H19, CDKNIC, PHLDA2, DLX5, ATP10A, SLC22A18 or TP73. Paternally expressed imprinted genes include but are not limited to IGF2, NDN, SNRPN, MEST, MAGEL2, and PEG3. Exemplary sequence information for these genes, including the human nucleic acid sequences, can be found at the geneimprint website (2006), available on the internet as of the filing date of the parent provisional application; this information is incorporated by reference herein.

“Telomere” refers to the sequences and the ends of a eukaryotic chromosome, consisting of many repeats of a short DNA sequence in specific orientation. Telomere functions include protecting the ends of the chromosome, so that chromosomes do not end up joined together, and allowing replication of the extreme ends of the chromosomes (by telomerase). The number of repeats of telomeric DNA at the end of a chromosome decreases with age and telomeres may play roles in aging and cancer. “Telomerase” refers to a DNA polymerase involved in the formation of telomeres and the maintenance of telomere sequences during chromosome replication.

By “parthenogenesis” or “parthenogenetic activation” is meant development of an oocyte or ovum without fusion of its nucleus with a male pronucleus to form a zygote. For example, an oocyte can be induced to divide without fertilization.

By “parthenote” is meant an oocyte or ovum that has been artificially activated. In some embodiments, the parthenote is totipotent. In some embodiments, a cell derived from a parthenote is pluripotent.

By “heterozygous parthenote” is meant a parthenote that has two different alleles of at least one gene. In one example, a heterozygous parthenote has two different alleles at about 30%, about 40%, about 50%, about 60%, about 70%, about 80% about 90% or at about 100% of the genetic loci.

By “homozygous parthenote” is meant a parthenote that does not have two different alleles of the same gene, so that it is homozyogous at 100% of the alleles. One example of a ES cell line that is derived from a homozygous parthenote is ORMES-9.

As used herein, the term “totipotent” or “totipotency” refers to a cell's ability to divide and ultimately produce an organism and its extraembryonic tissues in vivo. In one aspect, the term “totipotent” refers to the ability of the cell to progress through a series of divisions into a blastocyst in vitro. The blastocyst comprises an inner cellular mass (ICM) and a trophoblast. By ICM is meant the cells surrounded by the trophectoderm. The inner cell mass cells give rise to most of the fetal tissues upon further development. The cells found in the ICM give rise to pluripotent stem cells that possess the ability to proliferate indefinitely, or if properly induced, differentiate in all cell types contributing to an organism. By “trophectoderm” is meant the outermost layer of cells surrounding the blastocoel during the blastocyst stage of primate embryonic development. Trophectoderm becomes trophoblast and gives rise to most or all of the placental tissue upon further development. Trophoblast cells generate extra-embryonic tissues, including placenta and amnion.

As used herein, the term “pluripotent” refers to a cell's potential to differentiate into cells of the three germ layers: endoderm (e.g., interior stomach lining, gastrointestinal tract, the lungs), mesoderm (e.g., muscle, bone, blood, urogenital), or ectoderm (e.g., epidermal tissues and nervous system). Pluripotent stem cells can give rise to any fetal or adult cell type. Alone they cannot develop into a fetal or adult animal because they lack the potential to contribute to extraembryonic tissue (e.g., placenta in vivo or trophoblast in vitro).

Pluripotent stem cells (PSCs) are the source of multipotent stem cells (MPSCs) through spontaneous differentiation or as a result of exposure to differentiation induction conditions in vitro. The term “multipotent” refers to a cell's potential to differentiate and give rise to a limited number of related, different cell types. These cells are characterized by their multi-lineage potential and the ability for self-renewal. In vivo, the pool of multipotent stem cells replenishes the population of mature functionally active cells in the body. Among the exemplary multipotent stem cell types are hematopoietic, mesenchymal, or neuronal stem cells.

Transplantable cells include multipotent stem cells and more specialized cell types such as committed progenitors as well as cells further along the differentiation and/or maturation pathway that are partly or fully matured or differentiated. Exemplary transplantable cells include pancreatic, epithelial, cardiac, endothelial, liver, endocrine, and the like.

By “a cell derived from a parthenote” is meant a cell that results from the cell division of one or more cells in the parthenote. In some embodiments, a cell derived from a parthenote is a multipotent stem cell or a transplantable cell. In some embodiments, a cell derived from a parthenote has the same MEW haplotype as the parthenote.

By “paternally expressed gene” is meant a gene that is normally silenced by passage through the maternal germ line. Examples include, but are not limited to, PEG10, PLAGL1, DIRAS3, SGCE, IGF2, PEG3, MEST, or ZIM2.

By “maternally expressed gene” is meant a gene that is normally silenced by passage through the paternal germ line. Examples include, but are not limited to, TP73, PPP1R9A, DLX5, CPA4, CDKN1C, SLC22A18, GNAS, UBE3A, ATP10A, PHLDA2, or H19.

By “immortalized” is meant capable of undergoing at least 25, 50, 75, 90, or 95% more cell divisions than a naturally-occurring control cell of the same cell type, genus, and species as the immortalized cell or than the donor cell from which the immortalized cell was derived. Preferably, an immortalized cell is capable of undergoing at least 2, 5, 10, or 20-fold more cell divisions than the control cell. In one embodiment, the immortalized cell is capable of undergoing an unlimited number of cell divisions. Examples of immortalized cells include cells that naturally acquire a mutation in vivo or in vitro that alters their normal growth-regulating process. Other immortalized cells include cells that have been genetically modified to express an oncogene, such as ras, myc, abl, bcl2, or neu, or that have been infected with a transforming DNA or RNA virus, such as Epstein Barr virus or SV40 virus (Kumar et al., Immunol. Lett. 65:153 159, 1999; Knight et al., Proc. Nat. Acad. Sci. USA 85:3130 3134, 1988; Shammah et al., J. Immunol. Methods 160 19 25, 1993; Gustafsson and Hinkula, Hum. Antibodies Hybridomas 5:98 104, 1994; Kataoka et al., Differentiation 62:201211, 1997; Chatelut et al., Scand. J. Immunol. 48:659 666, 1998). Cells can also be genetically modified to express the telomerase gene (Rogues et al., Cancer Res. 61:8405 8507, 2001).

By “non-immortalized” is meant a cell that cannot divided indefinitely in vitro. In some embodiments, the non-immortalized cell does not have a nucleic acid mutation that alters its normal growth-regulating process. In some embodiments, the non-immortalized cell does not have two copies of the same recessive oncogene. In some embodiments, the non-immortalized cell cannot undergo 4-fold, 3-fold, 2-fold, or 1.5-fold more cell divisions in vitro and retain the same phenotype as the initial cell.

“An individual” as used herein intends a mammal, including but not limited to, a primate (e.g., a human, monkey, gorilla, ape, lemur, etc.), a bovine, an equine, a porcine, a canine, and a feline. Thus, the invention finds use in both human medicine and in the veterinary context, including use in agricultural animals and domestic pets. The individual may have been diagnosed with, is suspected of having, or is at risk of developing an indication. The individual may exhibit one or more symptoms associated with the indication. The individual can be genetically or otherwise predisposed to developing such a condition.

“Primate” refers to all animals in the primate order, including monkeys and humans. Exemplary non-human primates include, for example, chimpanzees, rhesus macaques, squirrel monkeys, lemurs. They include Old World, New World, and prosimian monkeys.

As used herein, “in need thereof” includes individuals who have a condition or disease or are “at risk” for the condition or disease. As used herein, an “at risk” individual is an individual who is at risk of development of a condition. An individual “at risk” may or may not have a detectable disease or condition, and may or may not have displayed detectable disease prior to the treatment methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). These risk factors include, but are not limited to, age, sex, race, diet, history of previous disease, presence of precursor disease, genetic (i.e., hereditary) considerations, and environmental exposure.

By “treatment” or “treating” is meant an approach for obtaining a beneficial or desired result, including clinical results. For purposes of this invention, beneficial or desired results include, but are not limited to, alleviation of symptoms associated with a condition diminishment of the extent of the symptoms associated with a condition, prevention of a worsening of the symptoms associated with a condition, or delaying the development of a disease or condition. In some embodiments, treatment with a one or more cells disclosed herein is accompanied by no or fewer side effects than are associated with currently available therapies.

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition. For example, the method may reduce the probability of disease development in a given time frame and/or reduce the extent of the disease in a given time frame, when compared to not using the method. In some embodiments, such comparisons are based on clinical studies using a statistically significant number of subjects. Disease development can be detectable using standard clinical techniques. Development may also refer to disease progression that can be initially undetectable and includes occurrence, recurrence, and onset.

The term “effective amount” is meant an amount of cells or an agent that achieves a desired effect, either in vivo or in vitro. In one example, an effective amount is an amount of one or more cells described herein which in combination with its parameters of efficacy and toxicity should be effective in a given therapeutic form based on the knowledge of the practicing specialist. As is understood in the art, an effective amount can be in one or more doses. As is understood in the clinical context, a therapeutically effective dosage of a cell or pharmaceutical composition may or may not be achieved in conjunction with another drug, compound, or pharmaceutical composition. Thus, an effective amount can be considered in the context of administering one or more therapeutic agents, and a single agent can be considered to be given in an effective amount if, in conjunction with one or more other agents, a desirable or beneficial result can be or is achieved.

By “pharmaceutically acceptable carrier” is meant any material which, when combined with an active ingredient, allows the ingredient to retain biological activity and does not provoke an unacceptable immune response (e.g., a severe allergy or anaphylactic shock) based on the knowledge of a skilled practitioner. Examples include, but are not limited to, any of the standard pharmaceutical carriers such as phosphate buffered saline solutions, water, emulsions such as oil/water emulsion, and various types of wetting agents. Exemplary diluents for aerosol or parenteral administration are phosphate buffered saline or normal (0.9%) saline. An exemplary carrier for the infusion of cells is a buminate/dextran solution. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, which are each hereby incorporated by reference in their entireties, particularly with respect to formulations).

By an “isolated” or “purified” means a cell or parthenote that has been separated from one or more components that are present when the cell is produced. In some embodiments, the cell is at least about 60%, by weight, free from other components that are present when the cell is produced. In various embodiments, the cell is at least about any of 75%, 90%, or 99%, by weight, pure. An isolated cell can be obtained, for example, by purification (e.g., extraction) from a natural source, fluorescence-activated cell-sorting, or other techniques known to the skilled artisan. Purity can be assayed by any appropriate method, such as fluorescence-activated cell-sorting. In some embodiments, the isolated cell is incorporated into a pharmaceutical composition of the invention or used in a method of the invention. The pharmaceutical composition of the invention may have additives, carriers, or other components in addition to the isolated cell.

It is understood that aspects and embodiments of the invention described herein include “comprising,” “consisting,” and “consisting essentially of” aspects and embodiments.

### Heterozygosity of Primate Parthenotes

Heterozygous primate parthenotes (e.g. human or non-human primate parthenotes) and multipotent stem cells or transplantable cells derived from them are disclosed herein. For example, Rhesus monkey parthenogenetic embryonic stem cell (RPESC) lines were generated that have unexpectedly high levels of heterozygosity at the majority of loci that are polymorphic in the oocyte donor. Human parthenogenetic embryonic stem cells can also be produced using the methods disclosed herein.

The high levels of genomic homozygosity anticipated in diploid, monoparental parthenogenetic ESCs represent a potential safety issue in the context of therapeutic engraftment, since widespread loss of heterozygosity is reportedly a common genetic alteration in human cancers. Thus, homozygous, parthenote-derived cells may carry multiple genetic defects because all of the recessive mutant alleles on the affected chromosome are unmasked, including alleles encoding tumor suppressors. However, in contrast to expectation, the stable diploid primate PESC lines described herein retained two different alleles in approximately 64% of the examined polymorphic loci. For example, several of the PESCs retained the heterozygosity at the major histocompatibility complex (WIC) region from the oocyte donor.

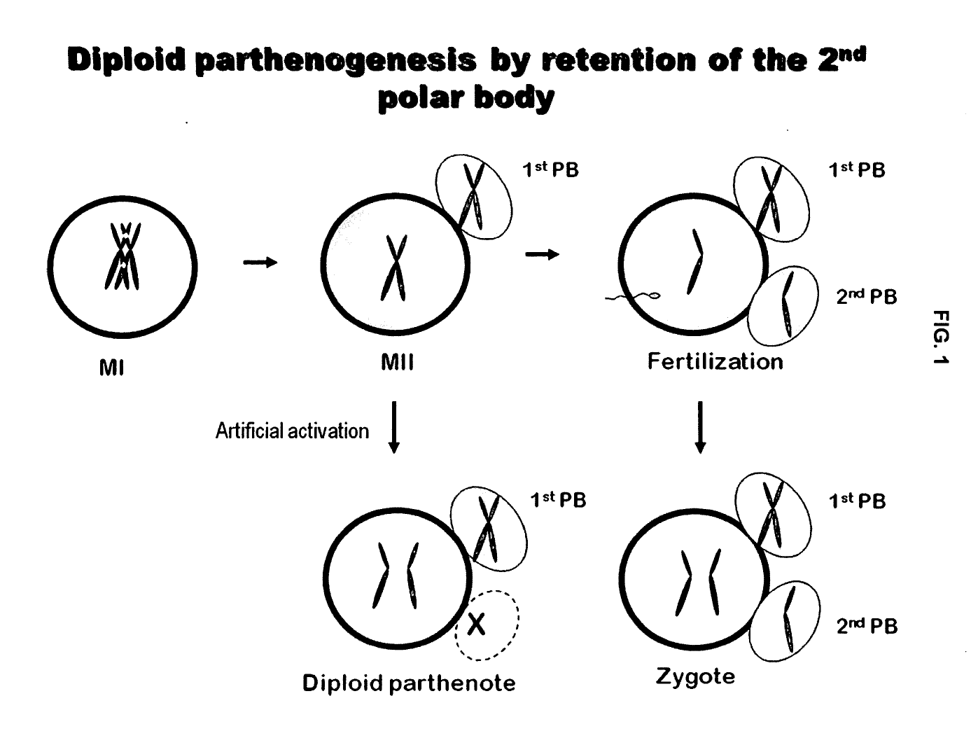

While not intending to be bound by any particular theory, this heterozygosity may be due to homologous recombination during the first meiotic division as illustrated in FIG. 1. In particular, in the methods disclosed herein homologous recombination can occur between homologous chromosomes in a primary oocyte during meiosis I (“MI” in FIG. 1). This results in a pair of heterozygous sister chromatids in the secondary oocyte in meiosis II (“MII” in FIG. 1). The other pairs of sister chromatids representing other chromosomes that are not shown may be homozygous (if no recombination occurs between them) or heterozygous (if recombination occurs between them). A first polar body contains the other set of sister chromatids from the oocyte. The secondary oocyte is then parthenogentically activated as described herein under conditions that prevent the formation of a second polar body. Thus, the resulting diploid parthenote has pairs of homologous sister chromatids, in which at least one pair of sister chromatids is heterozygous. In the absence of this homologous recombination at the first meiotic division, diploid parthenotes are expected to be homologous due to presence of pairs of identical sister chromatids. If the secondary oocyte is fertilized by a sperm instead of parthenogentically activated, a second polar body forms and the genetic material from the haploid oocyte is combined with that of the haploid sperm to produce a diploid zygote.

Due to the differences in the cellular machinery used by primates such as monkeys for homologous recombination and oocyte activation than that used by mice, the heterozygosity seen in the primate PESC lines described herein was not expected based on heterozygosity that has been reported for mouse parthenotes. Recent studies have shown a large variation in recombination and marked differences between humans and other species (Lynn, A., Ashley, T. & Hassold, T. Annu. Rev. Genomics Hum. Genet. 5, 317-349, 2004). In particular, it was unpredictable whether meiotic recombination would occur in primates at the first meiotic division. Additionally, the frequency of this homologous recombination that generated heterozygosity in ˜64% of the loci tested was much higher than would be expected. Homologous recombination and oocyte maturation are much similar among primates. Thus, highly heterozygous diploid parthenotes can be generated for other primates (e.g., humans) using the methods described that have been used to produce Rhesus monkeys parthenotes.

In some embodiments, the heterozygous parthenote has two different alleles for at least about any of 2, 3, 5, 10, 20, 40, 60, 80, 100, 150, 300, 400, 600, 1,000, 5,000, 10,000, or more genes. In some embodiments, the parthenote has two different alleles for at least about 1%, 2%, 5%, 10%, 15%, 20%, 25%, 30%, 40%, 50%, 60%, 65%, 70%, 75%, or 80% of the genes expressed by the parthenote. In some embodiments, the parthenote expresses two different alleles for at least 1%, 2%, 5%, 10%, 15%, 20%, 25%, 30%, 40%, 50%, 60%, 65%, 70%, 75%, or 80% of the genes expressed by the parthenote. In some embodiments, the heterozygous parthenote expresses more than one allele of a gene encoding an MEW molecule (e.g., an MEW Class 1 molecule such as HLA-A, HLA-B, or HLA-C; an MHC Class II molecule such as HLA-DPA1, HLA-DPB1, HLA-DQA1, HLA-DQB1, HLA-DRA, or HLA-DRB1; or an MEW Class III molecule). In particular embodiments, the heterozygous parthenote is heterozygous at least at one or more of the following MHC-linked microsatellite loci: D6S291, D6S2741, D6S2876, 9P06, DRA, MICA, 246K06, 162B17A, 162B17B, 151L13, MOGCA, 268P23, 222I18, D6S276, and D6S1691. Microsattelite DNA is a short sequence of di- or tri-nucleotide repeats of variable length. Methods are known to detect these sequences, such as by using PCR primers to the unique sequences upstream and downstream of a microsatellite DNA.

In some embodiments, the two nucleic acid alleles of at least about any of 2, 3, 5, 10, 20, 40, 60, 80, 100, 150, 300, 400, 600, 1000, or more alleles in a primate parthenote are less than about any of 99%, 95%, 90%, 80%, 70%, 60%, 50%, 40%, or 20% identical to each other. In some embodiments, the proteins encoded by the two alleles are of at least about any of 2, 3, 5, 10, 20, 40, 60, 80, 100, 150, 300, 400, 600, 1000, or more genes in a primate parthenote and are less than about any of 99%, 95%, 90%, 80%, 70%, 60%, 50%, 40%, or 20% identical to each other. In some embodiments, one allele of at least about any of 2, 3, 5, 10, 20, 40, 60, 80, 100, 150, 300, 400, 600, 1000, or more genes in a primate parthenote has at least about any of 1, 2, 3, 5, 10, 20, 30, 50, or more nucleic acid sequence differences (such as polymorphisms or mutations) or amino acid sequence differences in the encoded protein relative to the other allele of the same gene. In one embodiment, one allele differs from another allele by at least 1, 2, 3, 5, 10, 20, 30, 50, or more single nucleotide polymorphisms. Exemplary nucleic acid sequence differences include an insertion, deletion, frameshift mutation, silent mutation, nonsense mutation, or missense mutation. In some embodiments, the nucleic acid sequence difference is not a silent mutation. The two alleles can encode proteins variants. Exemplary protein sequence differences include the insertion of one or more amino acids (e.g., the insertion of 2, 3, 4, 5, 6, 7, 8, 9, or 10 amino acids), the deletion of one or more amino acids (e.g., a deletion of N-terminal, C-terminal, and/or internal residues, such as the deletion of at least about any of 5, 10, 15, 25, 50, 75, 100, 150, 200, 300, or more amino acids or a deletion of about any of 5, 10, 15, 25, 50, 75, 100, 150, 200, 300, or 400 amino acids), the replacement of one or more amino acids (e.g., the replacement of 2, 3, 4, 5, 6, 7, 8, 9, or 10 amino acids), or combinations of two or more of the foregoing. In some embodiments, an encoded protein has at least about any of 10%, 20%, 30%, 40%, 50%, 60%, 70%, 80%, 90%, 95%, or 100% of an activity of the corresponding protein encoded by the other allele of the same gene. Methods of detecting polymorphisms, including single nucleotide polymorphisms that can distinguish alleles are well known in the art.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

In some embodiments, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

### Relaxed Imprinting of Primate Parthenotes

In some embodiments, primate parthenotes (e.g. human or non-human primate parthenotes) are provided that express a paternally expressed gene or multipotent stem cells or transplantable cells derived from them. In particular, expression analysis of PESCs revealed transcripts from some imprinted genes that are normally expressed from paternal alleles. This result is striking since parthenotes only have maternal alleles, and thus are not expected to express paternally expressed genes. Expression of some paternally expressed genes may be desirable to increase the ability of a parthenote to differentiate into particular cell types (such as differentiation into desired cell types in vitro prior to cell transplantation or other applications or differentiation in vivo after cell transplantation). In various embodiments, a primate parthenote expresses any of about 2, 3, 4, 5, 6, 7, 8, 10, 15, 20, 30, 40, 50, 60, 80, 100, or more paternally expressed genes, such as PEG10, PLAGL1, DIRAS3, SGCE, IGF2, PEGS, MEST, ZIM2, or any combination of two or more of the foregoing.

Relaxed imprinting may not be detrimental for the production of a cell type of interest, and then the transplantation of multipotent stem cells or transplantable cells derived from parthenotes into patients. Indeed, monoparental, parthenogenetic and androgenetic stem cells in the mouse have been differentiated into transplantable hematopoietic progenitors in vitro that are reportedly capable of long-term multi-lineage reconstitution when engrafted into lethally irradiated adult mice. Moreover, in some cases postnatal monoparental chimeras are reportedly able to alleviate imprinting-related defects. Thus, if desired, primate parthenotes or multipotent stem cells or transplantable cells derived from them may be tested using standard methods (such as those described herein) to determine whether they have any abnormal growth patterns (such as uncontrolled growth) prior to therapeutic applications. Such cells may optionally be eliminated from further development as therapeutic agents. Cells that have normal growth patterns when tested in vitro or in animal models are expected to retain those normal growth patterns following transplantation into individuals, e.g., humans.

Strikingly, PESC lines produced both methylated and unmethylated signals from the IGF2 and H19 loci, although the level of methylation appeared reduced over bi-parental controls. While not intending to be bound by any particular theory, the culture conditions used to isolate and propagate PESC/ESC cells may alter methylation imprints.

### Totipotency and Pluripotency of Primate Parthenotes

Several of the isolated PESC lines were characterized. These new PESC lines express common pluripotency markers and posses other attributes of primate ESCs, including self renewal and the capacity to generate cell derivatives representative of all three germ layers in vivo and in vitro. In addition, the cells have telomere lengths similar to previously characterized embryonic stem cells, and longer than differentiated cells such as fibroblasts. The pluripotency of PESCs is desirable because it allows PESCs to be differentiated into a variety of cell types for therapeutic and research applications. It is noted that while the pluripotency of the PESC lines described herein appears similar to an existing PESC line (Cyno-1) from the cynomolgus macaque, the PESC lines described herein differ due to their heterozygosity in the MHC region and relaxed imprinting. The combination of pluripotency, heterozygosity (such as heterozygosity in the MHC region), long telomere length and relaxed imprinting makes cells derived from the primate parthenotes described herein particularly desirable for therapeutic and research applications.

In various embodiments, the primate parthenote is a totipotent or pluripotent primate cell that possess any one or more (including all) of the characteristic morphology of blast cells: high nuclear/cytoplasmic ratios, prominent nucleoli, and compact colony formation. A pluripotent cell is characterized by the presence of discrete cell surface markers or transcription factor expression that includes one or more (including all) of the following: OCT4, SSEA-3, SSEA-4, TRA-1-60, and TRA-1-81. These cells are also characterized by mRNA expression of all or one or more (including all) of the following: POU5F1 (OCT4), NANOG, SOX-2, TDGF, THY1, FGF4, TERT, and LEFTYA. The cells can also be characterized by the mRNA and/or protein expression of one or more (including all) of nuclear factor (erythroid-derived 2)-like 3 (NFE2L3), nuclear receptor subfamily 5, group A, member 2 (NR5A2), lymphocyte specific protein tyrosine kinase (LCK), V set domain containing T cell activation inhibitor 1 (VTCN1), developmental pluripotency associated 4 (DPPA4), solute carrier family 12 (SLC12A1), C14orf115, myosin VIIA and rab interacting protein (MYRIP), alcohol dehydrogenase 4 (ADH4) and PR-domain containing 14 (PRDM14) (see, for example, the GENECARD® website and iHOP® websites available on the internet).

In addition to marker and transcription factor expression profiles, the totipotent and pluripotent primate stem cells of the invention can also maintain a normal diploid karyotype. A normal karyotype is one where all chromosomes normally present in a species are present and have not been noticeably altered. Normal karyotype typically refers to the absence of chromosomal translocations, deletions, or insertions. The normal karyotype is readily determined by any method known to one of skill in the art, e.g., FISH for detecting translocation. In some embodiments, the totipotent and pluripotent primate stem cells of the invention have a karyotype that is stable throughout in vitro culturing. In addition, the karyotype remains stable even when the pluriopotent stem cells are cultured to differentiate into organ-specific cells and used for treatment purposes, e.g., transplantation. The totipotent and pluripotent cells have a telomere length that does not differ significantly from known totipotent and/or pluriopotent cells, and does differ significantly from the telomere length of differentiated cells such as fibroblasts. Statistical methods to determine if telomere length differs are well known in the art.

Totipotent parthenote cells as disclosed herein provide a source of pluripotent stem cells. The pluripotent stem cells can be propagated as a self-renewing cell line as well as provide a renewable source of multipotent stem cells and other transplantable cells. Pluripotent stem cells can differentiate under appropriate conditions into three embryonic germ layers; mesoderm (e.g., bone, cartilage, smooth muscle, striated muscle, and hematopoietic cells); endoderm (e.g., liver, primitive gut and respiratory epithelium); ectoderm (e.g., neurons, glial cells, hair follicles and tooth buds). The totipotent stem cells can also produce trophectodermal cells. One of skill in the art is familiar with how to assess the ability of pluripotent stem cells to differentiate into cells of the three germ layers. In one example, stem cells are implanted into an animal model, such as a nude mouse, and the cells are allowed to grow and form teratomas. After a suitable amount of time, the teratomas is removed, sectioned and stained to ascertain the layers that have formed. If the cell is totipotent or pluripotent, the resulting teratoma will contain tissues from each of the three germ layers.

In some embodiments, the totipotent stem cells provided herein act as a source of trophoblast and plurioptent stem cells which are capable of proliferating in vitro for at least 4 or more cell divisions while maintaining pluripotency.

In one embodiment, the pluripotent stem cells are capable of proliferating in vitro for at least 1 month or more, wherein the stem cell maintains its pluripotency. In other embodiments, the pluripotent stem cells are capable of proliferating in vitro for at least 2, 3, or 4 months or more, wherein the cell maintains its pluripotency. In other embodiments, the pluripotent stem cells are capable of proliferating in vitro for at least 5, 6, or 7 months or more, wherein the stem cell maintains its pluripotency. In another embodiment, the plurioptent stem cells are capable of proliferating in vitro for at least 8 months or more, wherein the stem cell maintains its pluripotency. In another embodiment, the plurioptent stem cells are capable of proliferating in vitro for at least 9 months or more, wherein the cell maintains its pluripotency. The methods of obtaining and culturing these cells are provided in greater detail below.

In one embodiment, the totipotent stem cells provided herein generate a blastocyst comprising an ICM and a trophoblast. The ICM serves as a source for the plurioptent stem cells, such as embryonic stem cells. Methods from the production of embryonic stem cells are known in the art.

### Efficiency of Primate Parthenote Generation

PESCs displaying both pluripotency and stable, diploid female karyotypes were generated with efficiencies comparable to those of sperm-fertilized controls. In particular, monkey parthenotes can be produced and cultured to the blastocyst-like stage and used in the isolation of pluripotent cell lines as efficiently as sperm-fertilized ESCs. Moreover, the establishment of three lines from oocytes recovered following a single ovarian stimulation cycle in one animal attests to the feasibility of employing PESCs as a source of pluripotent cells for the treatment of degenerative diseases. Thus, patient-specific PESCs can be a cost-effective medical option for autologous transplantation based on oocyte requirements, development rates, and overall PESC isolation efficiency.

### Exemplary Methods for Generating Primate Parthenotes

As described further herein, the invention provides methods of generating primate parthenotes by harvesting a primate oocyte and parthenogenetically activating it under conditions that produce a parthenote. One important aspect of the production of primate parthenotes is the use high quality oocytes. High quality oocytes can be obtained by using protocols that stimulate the animal (e.g., primates) to produce oocytes that are of high quality. Examples of such stimulation protocols are disclosed in the Examples and also in Zelinski-Wooten, et al. Hum. Reprod. 10:1658-1666 (1995). Another aspect that is important for ultimate success in getting totipotent or pluripotent stem cells is the method of harvesting.

In one aspect, when primates are stimulated to produce oocytes (e.g., hormonally) and these oocytes are harvested, the oocytes that are collected can be in different phases. Some oocytes are in metaphase I while other oocytes are in metaphase II. In such cases, the oocytes that are in metaphase I can be put into culture until they reach metaphase II and then used for parthenogenetic activation. Optionally, the oocytes that have been cultured to reach metaphase II are combined with the oocytes that were already at metaphase II when harvested for a pool of potential oocytes. In other cases, only the oocytes that are in metaphase II from the harvest are used for parthenogenetic activation. Any of these oocytes can be frozen for further use.

Harvested oocytes may be activated as described herein. For example, an oocyte can be activated by incubation in vitro with ionomycin under conditions sufficient to generate a primate parthenote that is heterozygous and/or expresses a paternally expressed gene.

In various embodiments, the concentration of ionomycin is between about 1 μM to about 20 μM, such between about 1 μM to about 10 μM, about 3 μM to about 7 μM, or about 5 μM to about 10 μM, or about 5 μM. In other example, the concentration of ionomycin is about 10 μM to about 15 μM, or about 15 μM to about 20 μM. In one example, the concentration of ionomycin is about 5 μM. In some embodiments, the concentration of ionomycin is about any of 1, 2, 3, 4, 5, 6, 7, 8, 9, or 10 μM. In various embodiments, the oocyte is incubated with ionomycin for between about 1 to about 20 minutes, such between about 1 to about 10 minutes, such as about 3 to about 7 minutes, or about 4 or about 5 minutes. However, the oocyte can be incubated with ionomycin for about 10 to about 15 minutes, or about 15 to about 20 minutes. In some embodiments, the oocyte is incubated with ionomycin for at least about any of 1, 2, 3, 4, 5, 6, 7, 8, 9, or 10 minutes. In some embodiments, the oocyte is incubated with ionomycin for about any of 1, 2, 3, 4, 5, 6, 7, 8, 9, or 10 minutes. In some embodiments, the oocyte is incubated in a medium (such as TALP/HEPES medium) with ionomycin and a first concentration of serum albumin (e.g., bovine or human serum albumin) as a first incubation.

In some embodiments, the method also includes incubating the oocyte in a medium (such as TALP/HEPES medium) with a second concentration of serum albumin (e.g., bovine or human serum albumin) that is greater than the first concentration of serum albumin (e.g., bovine or human serum albumin) as a second incubation. In some embodiments, the duration of the second incubation is between about 1 to about 20 minutes, such between about 1 to about 10 minutes, about 3 to about 7 minutes, or about 5 minutes. However, in other examples, the oocyte is incubated for about 5 to about 10 minutes, about 10 to about 15 minutes, or about 15 to about 20 minutes. In some embodiments, the duration of the second incubation is about any of 1, 2, 3, 4, 5, 6, 7, 8, 9, or 10 minutes. In particular embodiments, the first incubation is about 5 minutes in duration, and the second incubation is about 5 minutes in duration. In various embodiments, the first concentration of serum albumin (e.g., bovine or human serum albumin) is between about 0.1 mg/mL to about 10 mg/mL, such as bout 0.5 to about 5 mg/mL, or about 2 mg/mL. In additional embodiments, the first concentration of serum albumin is between about 0.1 mg/mL to about 0.5 mg/mL, about 0.5 mg/mL to about 1 mg/mL, about 1 mg/mL to about 2.5 mg/mL, about 2.5 mg/mL to about 5 mg/mL, about 5 mg/mL to about 7.5 mg/mL, or about 7.5 mg/mL to about 10 mg/mL. In particular embodiments, the first concentration of serum albumin (e.g., bovine or human serum albumin) is about any of 0.1, 0.5, 1, 2, 3, 4, 5, 6, 7, 8, 9, or 10 mg/mL. In various embodiments, the second concentration of serum albumin (e.g., bovine or human serum albumin) is about 10 mg/mL to about 60 mg/ml, such as about 20 to about 40 mg/mL, or about 30 mg/mL. In other embodiments, the second concentration of serum albumin is between about 10 mg/mL to about 20 mg/mL, about 20 mg/mL to about 30 mg/mL, about 30 mg/mL to about 40 mg/mL, about 40 mg/mL to about 50 mg/mL, or about 50 mg/mL to about 60 mg/mL. In particular embodiments, the second concentration of serum albumin (e.g., bovine or human serum albumin) is about any of 10, 20, 25, 30, 35, 40, 50, or 60 mg/mL. In some embodiments, the ratio of the first concentration of serum albumin to the second concentration of serum albumin is about any of 1:2, 1:3, 1:4, 1:5, 1:6, 1:10, 1:15, 1:20, 1:25, 1:30, 1:35, 1:40, 1:50, or 1:60. In some embodiments, the medium comprises 6-dimethylaminopurine. In some embodiments, the concentration of 6-dimethylaminopurine is between about 0.1 to about 10 mM, such as between about 0.5 μm to about 5 μM, such as about 1 μM to about 3 μM, or about 2 μM. In other examples, the concentration of 6-methylaminopurine is 0.1 mM to about 0.5 mM, about 0.5 mM to about 1 mM, about 1 mM to about 2.5 mM, about 2.5 mM to about 5 mM, about 5 mM to about 7.5 mM, or about 7.5 mM to about 10 mM. In some embodiments, the medium comprises as at least about 2 mM or comprises about 2 mM 6-dimethylaminopurine.

In some embodiments, the method further includes incubating the oocyte in a medium (such as HECM-9 medium) as a third incubation. In some embodiments, the duration of the third incubation is between about 1 to about 10 hours, such as about 2 to about 8 hours, such as about 3 to about 6 hours, or about 5 hours. In other examples, the third incubation is between about 1 to about 2.5 hours, about 2.5 to about 5 hours, about 5 to about 7.5 hours, or about 7.5 to about 10 hours. In particular embodiments, the duration of the third incubation is about any of 1, 2, 3, 4, 5, 6, 7, 8, 9, or 10 hours. In various embodiments, the third incubation is greater than 4 hours in duration, such as about any of 5, 6, 7, 8, 9, or 10 hours in duration. In some embodiments, the medium comprises 6-dimethylaminopurine. In some embodiments, the concentration of 6-dimethylaminopurine is about 0.1 to about 10 mM, such as between about 0.5 μm to about 5 μM, such as about 1 μM to about 3 μM, or about 2 μM. In additional embodiments, the concentration of 6-dimethylaminopurine is between about 0.1 to about 10 mM, such as between about 0.1 mM to about 0.5 mM, about 0.5 mM to about 1 mM, about 1 mM to about 2.5 mM, about 2.5 mM to about 5 mM, about 5 mM to about 7.5 mM, or about 7.5 mM to about 10 mM. In some embodiments, the medium comprises as at least about 2 mM or comprises about 2 mM 6-dimethylaminopurine. In some embodiments, the method also includes incubating the oocyte in a medium (such as HECM-9 medium) as a second incubation. In various embodiments, the medium does not include 6-demethylaminopurine but comprises cytochalasin B and either roscovitine or cycloheximide. In some embodiments, the concentration of cytochalasin B is between about between about 0.5 μg/mL to about 20 μg/mL, such as between about 1 μg/mL to about 20 μg/mL, 2 μg/mL to about 10 μg/mL or about 5 μg/mL cytochalsin B. In additional embodiments, the concentration of cytochalasin B is 0.5 μg/mL to about 1 μg/mL, about 1 μg/mL to about 2.5 μg/mL, about 2.5 μg/mL to about 5 μg/mL, about 5 μg/mL to about 7.5 μg/mL, about 7.5 μg/mL to about 10 μg/mL, about 10 μg/mL to about 12.5 μg/mL, about 12.5 μg/mL to about 15 μg/mL, or about 15 μg/mL to about 20 μg/mL. In some embodiments, the concentration of cytochalasin B is about any of 1, 2, 3, 4, 5, 6, 7, 8, 9, or 10 μg/mL. In particular embodiments, the concentration of cytochalasin B is about 5 μg/mL. In some embodiments, the concentration of roscovitine is between about 5 μM to about 200 μM, such as between about 5 μM to about 100 μM, 25 μM to about 75 μM, or about 50 μM roscotivine. In other embodiments, the concentration of roscovitine is about 5 μM to about 10 μM, about 10 μM to about 25 μM, about 25 μM to about 50 μM, about 50 μM to about 75 μM, about 75 μM to about 100 μM, about 100 μM to about 125 μM, about 125 μM to about 150 μM, or about 150 μM to about 200 μM. In some embodiments, the concentration of roscovitine is about any of 10, 20, 30, 40, 50, 60, 70, 80, 90, or 100 μM. In particular embodiments, the concentration of roscovitine is about 50 μM. In some embodiments, the concentration of cycloheximide is between about 0.5 μg/mL to about 20 μg/mL, 1 μg/mL to about 10 μg/mL, 5 μg/mL to about 8 μg/mL about 7 to about 8 μg/ml or about 7.7 μg/mL. In additional embodiments, the concentration of cycloheximide between about 0.5 μg/mL to about 1 μg/mL, about 1 μg/mL to about 2.5 μg/mL, about 2.5 μg/mL to about 5 μg/mL, about 5 μg/mL to about 7.5 μg/mL, about 7.5 μg/mL to about 10 μg/mL, about 10 μg/mL to about 12.5 μg/mL, about 12.5 μg/mL to about 15 μg/mL, or about 15 μg/mL to about 20 μg/mL. In some embodiments, the concentration of cycloheximide is about any of 1, 2, 3, 4, 5, 6, 7, 8, 9, 10, 12, 14, 16, 18, or 20 μg/mL. In particular embodiments, the concentration of cycloheximide is about 7.5 μg/mL. In several embodiments, roscovitine and cytoclasin B are used together, or cycloheximide and cyotchalasin B are used together.

In some embodiments, the third incubation occurs between about 20 to about 45° C., such as at about 37° C. In some embodiments, the third incubation occurs in air with a CO₂ concentration of between about 1 to about 15%, such as about 1% to about 10%, so between about 3% to about 7%, such as at about 5%. In other examples, the concentration is between about 1 to about 5%, about 5 to about 10%, or about 10 to about 15% (e.g., concentrations based on volume). In some embodiments, the third incubation occurs in air with a CO₂ concentration of about any of 1, 2, 3, 4, 5, 6, 7, 8, 9, 10, 12, or 15%. In some embodiments, the third incubation occurs in air with an O₂ concentration of between about 1 to about 15%, such as about 1% to about 10%, so between about 3% to about 7%, such as at about 5%. In other examples, the concentration is between about 1 to about 5%, about 5 to about 10%, or about 10 to about 15%. In some embodiments, the third incubation occurs in air with an O₂ concentration of about any of 1, 2, 3, 4, 5, 6, 7, 8, 9, 10, 12, or 15%. In some embodiments, the third incubation occurs in air with an N₂ concentration of between about 70 to about 98%, such as between about 80% to about 98%, or between about 85% to about 95%, such as about 90%. In other examples, the concentration is 70 to about 80%, about 80 to about 90%, or about 90 to about 98%. In some embodiments, the third incubation occurs in air with a N₂ concentration of about any of 70, 75, 80, 85, 90, 95, or 98%. In some embodiments, the third incubation occurs in air with a CO₂ concentration of about 5%, an O₂ concentration of about 5%, and an N₂ concentration of about 90%.

In some embodiments, the duration of the second incubation is between about 1 to about 10 hours, such between about 1 to about 2.5 hours, about 2.5 to about 5 hours, about 5 to about 7.5 hours, or about 7.5 to about 10 hours. In particular embodiments, the duration of the second incubation is about any of 1, 2, 3, 4, 5, 6, 7, 8, 9, or 10 hours. In various embodiments, the second incubation is greater than 4 hours in duration, such as about any of 5, 6, 7, 8, 9, or 10 hours in duration.

In certain embodiments, the oocyte is incubated with about 5 μM ionomycin in TALP/HEPES medium supplemented with about 1-3 mg/ml serum albumin for about 4-6 minutes as a first incubation. In particular embodiments, the oocyte is incubated with about 5 μM ionomycin in TALP/HEPES medium supplemented with about 1 mg/ml serum albumin for about 5 minutes as a first incubation. In some embodiments, the oocyte is then incubation in TALP/HEPES medium supplemented with about 28-32 mg/ml of serum albumin and about 1.5 to 2.5 mM dimethylamniopurine for about 4-6 minutes as a second incubation. In particular embodiments, the oocyte is incubation in TALP/HEPES medium supplemented with about 30 mg/ml of serum albumin and about 2 mM dimethylamniopurine for about 5 minutes as a second incubation. In some embodiments, the oocyte is then incubated in HECM-9 medium supplemented with about 1.5 to 2.5 mM dimethylamniopurine for about 4-6 hours as a third incubation. In particular embodiments, the oocyte is then incubated in HECM-9 medium supplemented with about 2 mM dimethylamniopurine for about 5 hours as a third incubation.

In certain embodiments, the oocyte is incubated with about 5 μM ionomycin in TALP/HEPES supplemented with about 1-3 mg/ml serum albumin for about 4-6 minutes as a first incubation. In particular embodiments, the oocyte is incubated with about 5 μM ionomycin in TALP/HEPES supplemented with about 1 mg/ml serum albumin for about 5 minutes as a first incubation. In some embodiments, the oocyte is then incubated in HECM-9 medium supplemented with (i) about 3-7 μg/mL cytochalasin B and (ii) about 30-70 μM roscovitine or about 5-10 μg/mL cycloheximide for about 4-6 hours as a second incubation. In particular embodiments, the oocyte is incubated in HECM-9 medium supplemented with (i) about 5 μg/mL cytochalasin B and (ii) about 50 μM roscovitine or about 7.5 μg/mL cycloheximide for about 5 hours as a second incubation.

In some embodiments, at least about any of 10%, 20%, 30%, 40%, 50%, 60%, 70%, 80%, 90%, 95%, or 100% of the parthenotes develop into cleaved embryos. In some embodiments, at least about any of 10%, 20%, 30%, 40%, 50%, 60%, 70%, 80%, 90%, 95%, or 100% of the cleaved embryos develop into an 8-cell embryo. In some embodiments, at least about any of 10%, 20%, 30%, 40%, 50%, 60%, 70%, 80%, 90%, 92%, 93%, 95%, or 100% of the cleaved embryos develop into a morula, such as a compact morula. In some embodiments, at least about any of 10%, 20%, 25%, 30%, 38%, 40%, 45%, 50%, 70%, or 80% of the cleaved embryos develop into a blastocyst.

Standard methods, such as those described herein, can be used to analyze primate parthenotes as well as multipotent stem cells and transplantable cells derived from them to determine whether they are heterozygous and thus contain at least one set of heterozygous alleles. For example, standard microsatellite/short tandem repeat (STR) analysis and/or standard single nucleotide polymorphism (SNP) analysis (such as the STR and SNP assays described herein) can be performed to determine whether genes of interest are homozygous or heterozygous in primate parthenotes or multipotent stem cells or transplantable cells derived from them (see, for example, Penedo et al., “Microsatellite typing of the rhesus macaque MHC region,” Immunogenetics, 57, 198-209, 2005).

Routine methods (such as those described herein) can also be used by a skilled artisan to determine whether primate parthenotes or multipotent stem cells or transplantable cells derived from them express parentally imprinted genes. For example, standard RT-PCR assays can be used to determine whether a parthenote, multipotent stem cell, or transplantable cell expresses particular parentally expressed genes (see, for example, Fujimoto et al., “Aberrant genomic imprinting in rhesus monkey embryonic stem cells,” Stem Cells, 24, 595-603. 2006). Microarray analyses, or assays that detect proteins (such as immunoassays) can also be utilized.

The totipotency and pluripotency of primate parthenotes can also be tested using standard methods (such as any of the methods described herein). For example, marker and transcription factor expression profiles can be used to determine if the parthenote has an expression pattern that is indicative of a totipotent or pluripotent cell. The ability of a primate parthenote to self renew and to generate cell derivatives representative of all three germ layers in vivo and in vitro can also be tested using routine techniques, such as those described herein (see, for example, Mitalipov, S., H and Kuo, H. C. et al. “Isolation and Characterization of Novel Rhesus Monkey Embryonic Stem Cell Lines,” Stem Cells, 24, 2177-86, 2006). In some embodiments, the primate parthenote generates a teratoma. In some embodiments, the primate parthenote generates representative of all three germ layers in vivo and/or in vitro.

If desired, primate parthenotes as well as multipotent stem cells and transplantable cells derived from them can be karyotyped with, for example, a standard G-banding technique (such as by the Cytogenetics Laboratory of the University of Wisconsin State Hygiene Laboratory, which provides routine karyotyping services) and compared to published karyotypes for the primate species.

Exemplary Compositions of Cells Derived from Primate Parthenotes

Multipotent stem cells or transplantable cells derived from a primate parthenote described herein may be used for the formulation of pharmaceutical or non-pharmaceutical compositions. As discussed herein, these formulations are useful in a variety of therapeutic and research applications.

In some embodiments, the pharmaceutical composition includes (i) one or more multipotent stem cells or transplantable cells derived from one or more primate parthenotes and (ii) a pharmaceutically acceptable carrier. In various embodiments, the cells are isolated or purified. While any suitable carrier known to those of ordinary skill in the art may be employed in the pharmaceutical compositions of this invention, the type of carrier will vary depending on the mode of administration. Compositions can be formulated for any appropriate manner of administration, including, for example, oral, intravenous, intra-arterial, intravesicular, inhalation, intraperitoneal, intrapulmonary, intramuscular, subcutaneous, intra-tracheal, transmucosal, intraocular, intrathecal, or transdermal administration. In some embodiments for oral administration, the cells are encapsulated so they can safely pass through the stomach. For parenteral administration, such as subcutaneous injection, the carrier may include, e.g., water, saline, alcohol, a fat, a wax, or a buffer. Biodegradable microspheres (e.g., polylactate polyglycolate) may also be used as carriers. In some embodiments, cells are administered directly into a tissue or organ, such as the bone marrow, brain, liver, kidney, pancreas, spleen, or other parenchymal organs.

In some embodiments, the pharmaceutical or non-pharmaceutical compositions include a buffer (e.g., neutral buffered saline, phosphate buffered saline, etc), a carbohydrate (e.g., glucose, mannose, sucrose, dextran, etc), an antioxidant, a chelating agent (e.g., EDTA, glutathione, etc.), a preservative, another compound useful for treating a condition, an inactive ingredient (e.g., a stabilizer, filler, etc), or combinations of two or more of the foregoing.

The compositions described herein may be administered as part of a sustained release formulation (e.g., a formulation such as a capsule or sponge that produces a slow release of cells following administration). In some embodiments, the cells are released over a period of about any of 4 hours, 8 hours, 12 hours, 16 hours, 1 day, 2 days, 3 days, 4 days, 5 days, 6 days, 1 week, 2 weeks, or more. In some embodiments, at least about any of 10%, 15%, 20%, 25%, 30%, 40%, 50%, 60%, 65%, 70%, 75%, 80%, 90%, 95%, 99%, or 100% of the released cells are viable. Such formulations may generally be prepared using well known technology and administered by, for example, rectal or subcutaneous implantation, or by implantation at the desired target site. Sustained-release formulations may contain cells dispersed in a carrier matrix and/or contained within a reservoir surrounded by a rate controlling membrane. Carriers for use within such formulations are biocompatible, and may also be biodegradable. In some embodiments, the formulation provides a relatively constant level of cell release. The amount of cells contained within a sustained release formulation depends upon the site of implantation, the rate and expected duration of release, and the nature of the condition to be treated or prevented.

It will be appreciated that the unit content of active ingredients contained in an individual dose of each dosage form need not in itself constitute an effective amount since the necessary effective amount could be reached by the combined effect of a plurality of administrations. The selection of the amount of cells to include in a pharmaceutical composition depends upon the dosage form utilized, the condition being treated, and the particular purpose to be achieved according to the determination of the ordinarily skilled artisan in the field.

To reduce or prevent an immune response in human subjects who are administered a pharmaceutical composition, the pharmaceutical composition may also include one or more immunosuppressive agents, such as cyclosporin. To bias the cells towards a desired cell type, the pharmaceutical composition may also include one or more growth factors, hormones, interleukins, cytokines, NGF, or other cells.

### Exemplary Methods for Generating Cells of a Desired Cell Type

The primate parthenotes described herein and cells derived from them are useful for the generation of cells of a desired cell type, e.g., for medical applications. In one such aspect, the invention features a method for producing a cell of a desired cell type by incubating an isolated or purified primate parthenote (e.g., a primate parthenote that is heterozygous and/or expresses a paternally expressed gene) or a cell derived from such a parthenote under in vitro conditions sufficient to generate a cell of a desired cell type.

As disclosed in U.S. Pat. No. 6,200,806, ESCs can be produced from human and non-human primate blastocysts. In one embodiment, primate ES cells are isolated “ES medium” that express SSEA-3; SSEA-4, TRA-1-60, and TRA-1-81 (see U.S. Pat. No. 6,200,806). ES medium consists of 80% Dulbecco's modified Eagle's medium (DMEM; no pyruvate, high glucose formulation, Gibco BRL), with 20% fetal bovine serum (FBS; Hyclone), 0.1 mM B-mercaptoethanol (Sigma), 1% non-essential amino acid stock (Gibco BRL). Generally, primate ES cells are isolated on a confluent layer of murine embryonic fibroblast in the presence of ES cell medium. In one example, embryonic fibroblasts are obtained from 12 day old fetuses from outbred mice (such as CF1, available from SASCO), but other strains may be used as an alternative. Tissue culture dishes treated with 0.1% gelatin (type I; Sigma) can be utilized. Distinguishing features of ES cells, as compared to the committed “multipotential” stem cells present in adults, include the capacity of ES cells to maintain an undifferentiated state indefinitely in culture, and the potential that ES cells have to develop into every different cell types. Dissociated cells are re-plated on embryonic feeder layers in fresh ES medium, and observed for colony formation. Colonies demonstrating ES-like morphology are individually selected, and split again as described above. The ES-like morphology is defined as compact colonies having a high nucleus to cytoplasm ratio and prominent nucleoli. Resulting ES cells are then routinely split manual disaggregation every 5-7 days as the cultures become dense. Early passage cells are also frozen and stored in liquid nitrogen. Cell lines can be karyotyped with a standard G-banding technique and compared to published karyotypes for the primate species.

In various embodiments, the desired cell type is an ectodermal cell, such as a stratified squamous epithelial, neural, or ganglion cell. In some embodiments, the desired cell type is a mesodermal cell, such as a cardiomyocyte or a cartilage, bone, muscle, fibrous connective tissue, or blood cell. In some embodiments, the desired cell type is an endodermal cell, such as an enteric-type columnar epithelial cell. Standard methods known in the art can be used to differentiate any of the primate parthenotes described herein into any desired cell type (see, for example, Mitalipov, S., H and Kuo, H. C. et al., “Isolation and Characterization of Novel Rhesus Monkey Embryonic Stem Cell Lines,” Stem Cells, 24, 2177-86, 2006; Kuo et al., “Differentiation of monkey embryonic stem cells into neural lineages,” Biol. Reprod. 2003; 68: 1727-1735, Notarianni et al. (eds.), Embryonic Stem Cells: A Practical Approach (Oxford University Press 2006); Lanza et al. (eds.) Handbook of Stem Cells (Academic Press 2004)).

Exemplary Therapeutic Applications of Cells Derived from Primate Parthenotes

Methods are provided for the treatment or prevention of disease in an individual (e.g., a mammal, such as a primate (e.g., a human, a monkey, a gorilla, an ape, a lemur, etc) that include administering a cell derived from a primate parthenote (e.g., any primate parthenote described herein) to the individual. For example, an effective amount of multipotent stem cells or transplantable cells derived from a primate parthenote can be administered to an individual (such as the oocyte donor or a relative of the oocyte donor) in need of one or more cell types to treat a disease, disorder, or condition without immunorejection.

Examples of diseases, disorders, or conditions that may be treated or prevented include neurological, endocrine, structural, skeletal, vascular, urinary, digestive, integumentary, blood, immune, auto-immune, inflammatory, endocrine, kidney, bladder, cardiovascular, cancer, circulatory, digestive, and muscular diseases, disorders, and conditions. Since many human diseases result from defects in a single cell type, replacing defective cells by cell or tissue replacement therapy using multipotent stem cells or transplantable cells derived from a primate parthenote can alleviate the symptoms of or cure various degenerative diseases. For example, the primate parthenotes described herein can be differentiated into cells such as pancreatic beta cells to treat diabetes or differentiated into cells such as substantia nigral dopaminergic neuronal cells to treat Parkinson's disease. Exemplary hematopoietic conditions include blood and immune conditions. In some embodiments, a hematopoietic stem cell derived from a primate parthenote is used to treat cancer. In some embodiments, these cells are used for reconstructive applications, such as for repairing or replacing tissues or organs. Other exemplary conditions include diseases of reproductive organs, skin, wound healing, and cosmetic conditions (such as hair loss, nails, etc).

In one such method, the invention features a procedure for treating a disease, disorder, or condition in an individual. In one embodiment, a multipotent stem cell or transplantable cell derived from a primate parthenote (e.g., a primate parthenote that is heterozygous and/or expresses a paternally expressed gene) is administered to an individual in need of one or more cell types. In some embodiments, a pharmaceutical composition that includes (i) one or more multipotent stem cells or transplantable cells derived from one or more primate parthenote and (ii) a pharmaceutically acceptable carrier is administered to the individual. In some embodiments, the oocyte used to generate the parthenote is from the same species as the individual being treated. In some embodiments, the oocyte used to generate the parthenote is from the individual being treated or a relative of the individual being treated. Exemplary sources of oocytes include a primate, such as a human, a monkey, a gorilla, an ape, or a lemur.

With respect to the therapeutic methods described herein, it is not intended that the administration of a cell derived from a primate parthenote to an individual be limited to a particular mode of administration, dosage, or frequency of dosing; the present invention contemplates all modes of administration, including intramuscular, intravenous, intraarticular, intralesional, subcutaneous, or any other route sufficient to provide a dose adequate to treat a condition. Both systemic and local administration is contemplated. The cells may be administered to the individual in a single dose or multiple doses. When multiple doses are administered, the doses may be separated from one another by, for example, one week, one month, one year, or ten years. One or more growth factors, hormones, interleukins, cytokines, small molecules, peptides, antibodies, or other cells may also be administered before, during, or after administration of the cells to further bias them towards a particular cell type. Additionally, one or more immunosuppressive agents, such as cyclosporin, may be administered to inhibit rejection of the transplanted cells. It is to be understood that, for any particular subject, specific dosage regimes should be adjusted over time according to the individual need and the professional judgment of the person administering or supervising the administration of the compositions.

### Exemplary Uses of Primate Parthenotes for Cell and Tissue Transplantation

Immunological responses to tissue and organ transplants vary depending on the extent of MEW antigen matching. ABO blood groups and three human MEW molecules including HLA-A, HLA-B, and HLA-DR are considered major factors in transplantation therapy. Blood type and MEW compatibility is associated with a reduced incidence of acute rejection, reduced need for anti-rejection therapy, and improved transplant survival (Taylor, C. J. et al., Lancet 366, 2019 (Dec. 10, 2005)). Analysis of the monkey PESCs described herein provides evidence that maternal MEW haplotypes are linked together during meiotic recombination resulting in either complete homozygosity (if no homologous recombination during the first meiotic division causes the exchange of MHC alleles between homologous chromosomes) or heterozygosity of PESCs (if homologous recombination during the first meiotic division causes the exchange of MHC alleles between homologous chromosomes). These results indicate that derivatives of PESCs can be used for autologous transplantation into oocyte donors or can be used to establish a bank of histocompatible cell lines for a broad spectrum of patients.

In particular, heterozygous lines that carry haplotypes identical to the egg donors should support autologous transplantation of PESC derivatives with no or limited rejection since the MEW molecules expressed by the transplanted cells are identical to those expressed by the oocyte donor. Cells that are heterozygous at one or more MEW alleles are more desirable for transplantation into the oocyte donor than other cells that have fewer alleles that are identical to those of the oocyte donor.

Cells that are homozygous at one or more MEW alleles express MEW molecules identical to those of the oocyte donor but will only express a portion of the MEW molecules expressed by the oocyte donor. If these cells are transplanted into the oocyte donor, the immune system may detect that some MEW molecules normally expressed by the oocyte donor are missing from the transplanted cells. Thus, in some embodiments, one or more immunosuppressive agents are administered to the oocyte donor to prevent rejection of the transplanted cells. The amount of immunosuppressive agent that is needed is expected to be far less than for the transplantation of cells that have fewer or no alleles that are identical to those of the oocyte donor. Thus, cells that are homozygous at one or more MEW alleles are also desirable for transplantation into the oocyte donor than other cells that have fewer alleles that are identical to those of the oocyte donor. Such cells can also be useful for transplantation into relatives of the oocyte donor or into other individuals that express one or more of the alleles that are expressed by the cells.

Homozygous PESC lines are also highly desirable for tissue matching and banking of pluripotent cell lines for use in many clinical applications. A recent estimate of the number of potential human ESC (hESC) lines required for various levels of HLA matching in the United Kingdom suggested that a bank of 150 diverse hESC lines would provide a full match at HLA-A, HLA-B, and HLA-DR for only a minority of recipients (<20%) registered on the UK kidney transplant waiting list (Taylor, C. J. et al., Lancet 366, 2019 (Dec. 10, 2005)). Moreover, according to calculation, extending the number of ESC lines beyond 150 would result in only an incremental benefit with respect to HLA matching. However, if hESC lines homozygous for common HLA haplotypes were available, only one ESC line with blood group O, homozygous for HLA-A1, HLA-B8, and HLA-DR3 would provide a complete HLA match for 13% of the recipient pool (Taylor, C. J. et al., Lancet 366, 2019 (Dec. 10, 2005)). Thus, only 10 highly selected ESC lines that are homozygous for MHC genes would provide an HLA-A, HLA-B, and HLA-DR match for 35-38% of recipients (Taylor, C. J. et al., Lancet 366, 2019 (Dec. 10, 2005); Faden, R. R. et al., Hastings Cent Rep 33, 13 (November-December, 2003)). Therefore, the creation of PESC lines that are highly homozygous at MHC loci is an attractive and realistic approach to generating a “histocompatibility bank” since it requires significantly fewer cell lines for MHC matching. The RPESC-2 and Cyno-1 cell lines show complete homozygosity within all 15 STRs and therefore satisfy the requirements for such cells.

Multipotent stem cells or transplantable cells derived from the parthenotes described herein may also be used to generate an immunological reaction that stimulates the destruction of tumors. In particular, graft-versus-host disease often correlates with graft-versus-tumor reactions and thus to better survival of cancer patients. Therefore, cells that express some but not all of the alleles expressed by the patient (such as cells that are homozygous for MHC alleles and are transplanted into the oocyte donor) may be used to stimulate an immune response that is sufficient to increase the destruction of tumor cells by the immune system but is not as strong as the immune response generated by cells that do not express any MHC molecules identical to those expressed by the patient. Thus, the immune response is not strong enough to generate a clinically unacceptable level of adverse effects.

Exemplary Libraries of Primate Parthenotes and Cells Derived from Them

Libraries are provided that include one or more primate parthenotes described herein or cells derived from them (such as multipotent stem cells or transplantable cells). The libraries are useful as cell banks for cell transplantation applications. In some embodiments, the library provides an HLA-A, HLA-B, and HLA-DR match for at least about any of 5%, 10%, 20%, 30%, 40%, 50%, 60%, 70%, 80%, 85%, 90%, 95%, 99%, or 100% of the potential recipients. For example, the library may provide at least about any of 5%, 10%, 20%, 30%, 40%, 50%, 60%, 70%, 80%, 85%, 90%, 95%, 99%, or 100% of the HLA-A, HLA-B, and HLA-DR alleles used for MHC matching. In various embodiments, the library includes at least about any of 5, 10, 15, 20, 50, 100, 200, 500, 1,000, or more parthenotes or cells. Such large libraries can be obtained due to high efficiency achieved by the methods described herein for generating such parthenotes and cells derived from them.

In some embodiments, the library includes any one, two, three, or four of the following: (i) a primate parthenote or cell (e.g., a multipotent stem cell or transplantable cell derived from a parthenote) that has a set of heterozygous WIC alleles and a set of heterozygous alleles for a gene that does not encode an WIC molecule, (ii) a primate parthenote or cell (e.g., a multipotent stem cell or transplantable cell derived from a parthenote) that has a set of heterozygous WIC alleles and a set of homozygous alleles for a gene that does not encode an WIC molecule, (iii) a primate parthenote or cell (e.g., a multipotent stem cell or transplantable cell derived from a parthenote) that has a set of homozygous WIC alleles and a set of homozygous alleles for a gene that does not encode an WIC molecule, or (iv) a primate parthenote or cell (e.g., a multipotent stem cell or transplantable cell derived from a parthenote) that has a set of homozygous WIC alleles and a set of heterozygous alleles for a gene that does not encode an WIC molecule. The library may have additional parthenotes or cells (such as multipotent stem cells or transplantable cells derived from a parthenote).

In some embodiments, the library includes three or more isolated or purified primate parthenotes or cells derived from an isolated or purified primate parthenote. In particular embodiments, the first parthenote or cell has a set of heterozygous WIC alleles and a set of heterozygous alleles for a gene that does not encode an WIC molecule. In particular embodiments, the second parthenote or cell has a set of heterozygous MEW alleles and a set of homozygous alleles for a gene that does not encode an MEW molecule. In particular embodiments, the third parthenote or cell has a set of homozygous MEW alleles and a set of homozygous alleles for a gene that does not encode an MEW molecule. In some embodiments, the library further includes a fourth isolated or purified primate parthenote or cell derived from an isolated or purified primate parthenote. In particular embodiments, the fourth primate or cell has a set of homozygous MEW alleles and a set of heterozygous alleles for a gene that does not encode an MEW molecule.

In some embodiments, the library includes three or more isolated or purified primate parthenotes or cells derived from an isolated or purified primate parthenote. In particular embodiments, the first parthenote or cell has a set of heterozygous MEW alleles and a set of heterozygous alleles for a gene that does not encode an MEW molecule. In particular embodiments, the second parthenote or cell has a set of heterozygous MEW alleles and a set of homozygous alleles for a gene that does not encode an MEW molecule. In particular embodiments, the third parthenote or cell has a set of homozygous MEW alleles and a set of heterozygous alleles for a gene that does not encode an MEW molecule.

In some embodiments, the library includes two or more isolated or purified primate parthenotes or cells derived from an isolated or purified primate parthenote. In particular embodiments, the first parthenote or cell has a set of heterozygous MEW alleles and a set of heterozygous alleles for a gene that does not encode an MEW molecule. In particular embodiments, the second parthenote or cell has a set of homozygous MEW alleles and a set of heterozygous alleles for a gene that does not encode an MEW molecule. In some embodiments, the second parthenote or cell has a set of homozygous MEW alleles and a set of homozygous alleles for a gene that does not encode an MEW molecule.

In some embodiments, the library includes two or more isolated or purified primate parthenotes or cells derived from an isolated or purified primate parthenote. In particular embodiments, the first parthenote or cell has a set of heterozygous MEW alleles and a set of homozygous alleles for a gene that does not encode an MEW molecule. In particular embodiments, the second parthenote or cell has a set of homozygous MHC alleles and a set of heterozygous alleles for a gene that does not encode an MHC molecule. In some embodiments, the second parthenote or cell has a set of homozygous MHC alleles and a set of homozygous alleles for a gene that does not encode an MHC molecule.

In some embodiments, the library includes two or more isolated or purified primate parthenotes or cells derived from an isolated or purified primate parthenote. In particular embodiments, the first parthenote or cell has a set of homozygous MHC alleles and a set of heterozygous alleles for a gene that does not encode an MHC molecule. In particular embodiments, the second parthenote or cell is a different parthenote or cell that also has a set of homozygous MHC alleles and a set of heterozygous alleles for a gene that does not encode an MHC molecule. In some embodiments, the second parthenote or cell has a set of homozygous MHC alleles and a set of homozygous alleles for a gene that does not encode an MHC molecule. In particular embodiments, the second parthenote or cell has a set of heterozygous MHC alleles and a set of homozygous alleles for a gene that does not encode an MHC molecule. In particular embodiments, the second parthenote or cell has a set of heterozygous MHC alleles and a set of heterozygous alleles for a gene that does not encode an MHC molecule.

In some embodiments of any of the libraries, a parthenote or cell with a set of heterozygous MHC alleles is heterozygous for HLA-A, HLA-B, HLA-DR, or any two or more of the foregoing alleles. In some embodiments of any of the libraries, a parthenote or cell with a set of heterozygous MHC alleles is heterozygous for HLA-A, HLA-B, and HLA-DR alleles. In some embodiments of any of the libraries, a parthenote or cell with a set of heterozygous MHC alleles is heterozygous for one or more of the following MEW-linked microsatellite loci: D6S291, D6S2741, D6S2876, 9P06, DRA, MICA, 246K06, 162B17A, 162B17B, 151L13, MOGCA, 268P23, 222I18, D6S276, and D6S1691. In some embodiments of any of the libraries, at least about any of 1, 2, 5, 10, 20, 40, 50, 60, 80, 100, 200, 500, or more parthenotes or cells express a paternally expressed gene. In various embodiments of any of the libraries, at least about any of 1, 2, 5, 10, 20, 40, 50, 60, 80, 100, 200, 500, or more parthenotes or cells express at least about any of 2, 5, 10, 20, 40, 60, 80, 100, 200, or more parentally imprinted genes. In additional embodiments, the library includes any of the parthenotes or cells described above.

### Exemplary Uses of Primate Parthenotes for Research and Drug Screening Applications

The primate parthenotes described herein and cells derived from them can be used in a variety of research and drug screening applications. Meiotic recombination is an important evolutionary force that plays a vital role in shaping genomes and creating diversity. Recent explorations suggest that meiotic recombination events tend to happen in certain regions of the genome, leading to the concept of recombination hot spots (Jeffreys, A. J. et al. Hum Mol Genet. 14, 2277 (Aug. 1, 2005)). Direct analysis of fine-scale recombination events is not feasible by pedigree analysis. The only information on recombination in primates comes from single molecule PCR analysis of sperm. However, recombination frequencies may be gender dependent. Crossing-over is estimated to occur approximately fifty-five times during meiosis in males, and about seventy-five times in females (Jeffreys, A. J. et al. Hum Mol Genet. 14, 2277 (Aug. 1, 2005)). Thus, the primate parthenotes described herein and cells derived from them can be used for the analysis of recombination in the primate female (e.g., human females). Each PESC line represents the recombination events that occurred within a single oocyte. Thus primate PESCs, in combination with standard genetic and cytological assays, are valuable in vitro models to study increasingly complex aspects of meiosis, chromosome behavior, recombination, and genomic imprinting. It is generally accepted that exchange during meiotic recombination occurs more frequently at distal telomeric regions of chromosomes with the frequency of recombination decreasing near the centromeric region. In murine PESC lines, evidence has been presented that the recombination rate is directly proportional to the distance from the centromere (Kim, K. et al., Science (Dec. 14, 2006)). The recently developed genetic linkage map and whole genome DNA sequencing can be used for precise mapping of centromeres in macaque (Gibbs, R. A. and Rogers, J. et al. (2007). “Evolutionary and biomedical insights from the rhesus macaque genome,” Science 316(5822): 222-34; Ventura, M. and Antonacci, F. et al. (2007). “Evolutionary formation of new centromeres in macaque,” Science 316(5822): 243-6). The location of the chromosomal centromeres in the Rhesus monkey can be used in the analysis of recombination frequencies. The primate parthenotes described herein and cell derived from them can also be used in screening assays to identify candidate compounds that modulate cell division (e.g., meiosis or mitosis), chromosome behavior, recombination (e.g., homologous recombination), genomic imprinting, self-renewal, differentiation, maturation, migration, or any two or more of the foregoing.

For example, these cells can be used to determine the effect of candidate compounds on cell division (e.g., meiosis or mitosis), chromosome behavior, recombination (e.g., homologous recombination), genomic imprinting, self-renewal, differentiation, maturation, migration, or any two or more of the foregoing. In some embodiments, a primate parthenote (e.g., a primate parthenote that is heterozygous and/or expresses a paternally expressed gene) or a cell derived from a primate parthenote is contacted with a candidate compound, and cell division, chromosome behavior, recombination, genomic imprinting, self-renewal, differentiation, maturation, or migration, or any two or more of the foregoing is measured or assayed. The candidate compound is determined to modulate cell division, chromosome behavior, recombination, genomic imprinting, self-renewal, differentiation, maturation, or migration if the candidate compound causes a change in cell division, chromosome behavior, recombination, genomic imprinting, respectively.

Cell proliferation can be studied by evaluating the cell cycle using fluorescence-activated cell-sorting (FACS). Mechanisms regulating self-renewal of stem cells and the differentiation of stem cells can be studied using standard methods. Various compounds and incubation conditions can be tested to determine conditions that maintain a pool of stem cells by supporting their self-renewal. Compounds and incubation conditions can also be tested to determine conditions that induce the differentiation of stem cells, including the differentiation of cancer stem cells. Mechanisms that regulate migration of stem cells and their derivatives are also important for transplantation strategies. Numerous compounds can be tested, such as peptide libraries, antibody libraries, small molecule libraries, etc. These approaches can be useful for all aspects of regenerative medicine.

Exemplary test agents that can be screened include, but are not limited to, peptides such as, soluble peptides, including but not limited to members of random peptide libraries (see, e.g., Lam et al., Nature, 354:82-84, 1991; Houghten et al., Nature, 354:84-86, 1991), and combinatorial chemistry-derived molecular library made of D- and/or L-configuration amino acids, phosphopeptides (including, but not limited to, members of random or partially degenerate, directed phosphopeptide libraries; see, e.g., Songyang et al., Cell, 72:767-778, 1993), antibodies (including, but not limited to, polyclonal, monoclonal, humanized, anti-idiotypic, chimeric or single chain antibodies, and Fab, F(ab')₂ and Fab expression library fragments, and epitope-binding fragments thereof), small organic or inorganic molecules (such as, so-called natural products or members of chemical combinatorial libraries), molecular complexes (such as protein complexes), or nucleic acids.

Appropriate agents can be contained in libraries, for example, synthetic or natural compounds in a combinatorial library. Numerous libraries are commercially available or can be readily produced; means for random and directed synthesis of a wide variety of organic compounds and biomolecules, including expression of randomized oligonucleotides, such as antisense oligonucleotides and oligopeptides, also are known. Alternatively, libraries of natural compounds in the form of bacterial, fungal, plant and animal extracts are available or can be readily produced. Additionally, natural or synthetically produced libraries and compounds are readily modified through conventional chemical, physical and biochemical means, and may be used to produce combinatorial libraries. Such libraries are useful for the screening of a large number of different compounds.

Libraries of agents to be screened (such as combinatorial chemical libraries) useful in the disclosed methods include, but are not limited to, peptide libraries (see, e.g., U.S. Pat. No. 5,010,175; Furka, Int. J. Pept. Prot. Res., 37:487-493, 1991; Houghton et al., Nature, 354:84-88, 1991; PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Natl. Acad. Sci. USA, 90:6909-6913, 1993), vinylogous polypeptides (Hagihara et al., J. Am. Chem. Soc., 114:6568, 1992), nonpeptidal peptidomimetics with glucose scaffolding (Hirschmann et al., J. Am. Chem. Soc., 114:9217-9218, 1992), analogous organic syntheses of small compound libraries (Chen et al., J. Am. Chem. Soc., 116:2661, 1994), oligocarbamates (Cho et al., Science, 261:1303, 1003), and/or peptidyl phosphonates (Campbell et al., J. Org. Chem., 59:658, 1994), nucleic acid libraries (see Sambrook et al. Molecular Cloning, A Laboratory Manual, Cold Springs Harbor Press, N.Y., 1989; Ausubel et al., Current Protocols in Molecular Biology, Green Publishing Associates and Wiley Interscience, N.Y., 1989), peptide nucleic acid libraries (see, e.g., U.S. Pat. No. 5,539,083), antibody libraries (see, e.g., Vaughn et al., Nat. Biotechnol., 14:309-314, 1996; PCT App. No. PCT/US96/10287), carbohydrate libraries (see, e.g., Liang et al., Science, 274:1520-1522, 1996; U.S. Pat. No. 5,593,853), small organic molecule libraries (see, e.g., benzodiazepines, Baum, C&EN, Jan 18, page 33, 1993; isoprenoids, U.S. Pat. No. 5,569,588; thiazolidionones and methathiazones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514) and the like.

Libraries of agents useful for the disclosed screening methods can be produce in a variety of manners including, but not limited to, spatially arrayed multipin peptide synthesis (Geysen, et al., Proc. Natl. Acad. Sci., 81(13):3998-4002, 1984), “tea bag” peptide synthesis (Houghten, Proc. Natl. Acad. Sci., 82(15):5131-5135, 1985), phage display (Scott and Smith, Science, 249:386-390, 1990), spot or disc synthesis (Dittrich et al., Bioorg. Med. Chem. Lett., 8(17):2351-2356, 1998), or split and mix solid phase synthesis on beads (Furka et al., Int. J. Pept. Protein Res., 37(6):487-493, 1991; Lam et al., Chem. Rev., 97(2):411-448, 1997). Libraries may include a varying number of compositions (members), such as up to about 100 members, such as up to about 1000 members, such as up to about 5000 members, such as up to about 10,000 members, such as up to about 100,000 members, such as up to about 500,000 members, or even more than 500,000 members.

In one convenient embodiment, high throughput screening methods involve providing a combinatorial chemical or peptide library containing a large number of potential therapeutic compounds. Such combinatorial libraries are then screened in one or more assays as described herein to identify those library members (particularly chemical species or subclasses) that display a desired characteristic activity. The compounds identified using the methods disclosed herein can serve as conventional “lead compounds” or can themselves be used as potential or actual therapeutics. In some instances, pools of candidate agents may be identify and further screened to determine which individual or subpools of agents in the collective have a desired activity.

Exemplary Kits with Cells Derived from Primate Parthenotes

Also provided are articles of manufacture and kits that include one or more cells derived from any of the primate parthenotes described herein (e.g., primate parthenotes that are heterozygous and/or express a paternally expressed gene) and suitable packaging. In some embodiments, a kit is provided that includes (i) one or more multipotent stem cells or transplantable cells derived from one or more primate parthenotes and (ii) instructions for using the kit to treat a condition in an individual. In various embodiments, a kit is provided with (i) one or more multipotent stem cells or transplantable cells derived from one or more primate parthenotes and (ii) instructions for using the kit for any of the research or drug screening uses described herein.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use cells derived from primate parthenotes generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of primate parthenotes or cells derived from them to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of cells and instructions for use and packaged in quantities sufficient for storage and use in pharmacies, for example, hospital pharmacies and compounding pharmacies.

### General Techniques

The practice of the present invention employees many techniques of stem cell biology, cell culturing, molecular biology (including recombinant techniques), microbiology, cell biology, biochemistry and immunology. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Animal Cell Culture (R. I. Freshney), ed., 1987); Methods in Enzymology (Academic Press, Inc.); Handbook of Experimental Immunology (D. M. Weir &C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) Short Protocols in Molecular Biology (Wiley and Sons, 1999), Embryonic Stem Cells: A Practical Approach (Notaranni et al. eds., Oxford University Press 2006); and Essential of Stem Cell Biology (R. Lanza, ed., Elsevier Academic Press 2006).

### EXAMPLES

The examples, which are intended to be purely exemplary of the invention and should therefore not be considered to limit the invention in any way, also describe and detail aspects and embodiments of the invention discussed above. Unless indicated otherwise, temperature is in degrees Centigrade and pressure is at or near atmospheric.

### Example 1

### Production and Characterization of Diploid Heterozygous Parthenogenetic Embryos and Cells Derived from Them

### Rhesus Monkey PESC Lines

### Ovarian Stimulation and Recovery of Rhesus Monkey Oocytes

Oocytes were collected using standard methods (Mitalipov S M, Wolf D P. “Nuclear transfer in non-human primates. In: Nuclear transfer protocols: Cell reprogramming and transgenesis.” Methods in Molecular Biology. Human Press. Edited by Verma P J and Trounson A O. June 2006; ISBN: 1-58829-280-0). For controlled ovarian stimulation and oocyte recovery, previously described methods were modified (see, for example, Zelinski-Wooten, M. B., J. S. Hutchison, et al. (1995). “Follicle stimulating hormone alone supports follicle growth and oocyte development in gonadotrophin-releasing hormone antagonist-treated monkeys.” Hum Reprod 10(7): 1658-66). Cycling females were subjected to follicular stimulation using twice-daily intramuscular injections of recombinant human FSH as well as concurrent treatment with Antide, a GnRH antagonist, for 8-9 days. Females received recombinant human LH on days 7-9 and recombinant HCG on day 10. Cumulus-oocyte complexes were collected from anesthetized animals by laparoscopic follicular aspiration (28-29 hours post hCG) and placed in Hepes-buffered TALP (modified Tyrode solution with albumin, lactate, and pyruvate) medium (Bavister, B. D. and Yanagimachi (1977). “The effects of sperm extracts and energy sources on the motility and acrosome reaction of hamster spermatozoa in vitro.” Biol Reprod 16(2): 228-37) containing 0.3% BSA (TH3) at 37° C. Unless indicated otherwise, all reagents were from Sigma-Aldrich Co. (St. Louis, Mo.). Oocytes, stripped of cumulus cells by mechanical pipetting after brief exposure (<1 minute) to hyaluronidase (0.5 mg/ml), were placed in chemically defined, protein-free HECM-9 medium (McKiernan, S. H. and B. D. Bavister (2000), “Culture of one-cell hamster embryos with water soluble vitamins: pantothenate stimulates blastocyst production.” Hum Reprod 15(1): 157-64) at 37° C. in 5% CO₂, 5% O₂, and 90% N₂ until further use.

### Materials

1. Recombinant human FSH, LH and CG (Ares Advanced Technologies Inc.; Norwell, Mass.) (or recombinant monkey gonadotropins when available)

2. Antide (GnRH antagonist, Ares Advanced Technologies Inc.)

3. Ketamine (Vedco, Inc., St. Joseph, Mo.)

4. TH3 medium: Hepes-buffered TALP medium, containing 0.3% BSA. The medium was prepared by adding the indicated amounts of each reagent (Sigma, St. Louis, Mo.) to 1 L of Milli-Q water.

| NaCl | 6.660 g |
|---|---|
| KCl | 0.239 g |
| CaCL₂—2H₂O | 0.294 g |
| MgCl₂—6H₂O | 0.102 g |
| Na₂HPO₄ | 0.048 g |
| Glucose | 0.900 g |
| Na Lactate | 1.87 ml |
| Phenol Red | 0.010 g |
| NaHCO₃ | 0.168 g |
| Gentamicin sulfate | 0.050 g |
| Hepes | 2.603 g |
| Na Pyruvate | 0.060 g |
| pH | 7.2-7.4 |
| Osmolarity | 282 ± 10 |

The medium was filtered using a 0.2μ filter unit and stored for up to one month at +4° C. Then, BSA (Sigma) was added at 3 mg/ml prior to use and refiltered.

5. HECM-9 medium: HECM-9 base medium was prepared by adding the indicated amounts of each reagent (Sigma) to 1 L of Milli-Q water.

| | | |
|---|---|---|
| a. PVA | 0.1 | g |
| b. NaCl | 6.639 | g |
| c. KCl | 0.224 | g |
| d. CaCl₂•2H₂O | 0.279 | g |
| e. MgCl₂•6H₂O | 0.102 | |
| f. NaHCO₃ | 2.1 | g |
| g. Lactic Acid, Na salt, 60% syrup | 632 | μl |
| h. Gentamicin sulfate | 0.01 | g |

| | |
|---|---|
| i. pH | 7.2-7.4 |
| j. Osmolarity | 277 ± 5 |

The medium was filtered using a 0.2μ filter unit and stored for up to one week at +4° C.

6. 100× Amino Acid/Pantothenate stock: The stock was prepared by adding the indicated amounts of each reagent (Sigma) to 1 L of Milli-Q water.

TABLE 8
100 x Amino Acid/Pantothenate Stock for HECM-9

| | Sigma | Final | | | | | |
|---|---|---|---|---|---|---|---|
| Component | Catalog# | Concentration | | FW | mg/10 ml | mg/100 ml | mg/1000 ml |
| Taurine | T-7146 | 0.50 | mM | 125.1 | 62.6 | 626.0 | 6260.0 |
| Asparagine | A-4159 | 0.01 | mM | 132.1 | 1.3 | 13.0 | 130.0 |
| Cysteine | C-8277 | 0.01 | mM | 175.6 | 1.8 | 18.0 | 180.0 |
| Histidine | H-9511 | 0.01 | mM | 209.6 | 2.1 | 21.0 | 210.0 |
| Lysine | L-1262 | 0.01 | mM | 182.6 | 1.8 | 18.0 | 180.0 |
| Proline | P-4655 | 0.01 | mM | 115.1 | 1.2 | 12.0 | 120.0 |
| Serine | S-5511 | 0.01 | mM | 105.1 | 1.1 | 11.0 | 110.0 |
| Aspartic | A-4534 | 0.01 | mM | 133.1 | 1.3 | 13.0 | 130.0 |
| Acid | | | | | | | |
| Glycine | G-6388 | 0.01 | mM | 75.07 | 0.8 | 8.0 | 80.0 |
| Glutamic | G-5889 | 0.01 | mM | 169.1 | 1.7 | 17.0 | 170.0 |
| Acid | | | | | | | |
| Glutamine | G-5763 | 0.20 | mM | 146.1 | 29.2 | 292.0 | 2920.0 |
| Pantothenic | P-5155 | 3.0 | μM | 238.3 | 0.7 | 7.0 | 70.0 |
| Acid | | | | | | | |

This stock was filtered and distributed as 500 μl per 1.5 ml tubes and stored at −20° C. for up to three months. The aliquots were thawed immediately before use. Sigma replaced the specific catalogue number of cell culture grade amino acids with biotechnology grade. These new amino acids have been tested for monkey embryo growth.

7. HECM-9 complete medium: AA/Pantothenate stock was added to HECM-9 base medium at a ratio of 1:100 prior to use (100 μl stock to 10 ml HECM-9 medium). HECM-9aa was used to hold oocytes from the time of recovery until activation, as well as to culture embryos until the 4-8-cell stage (or Day 2). For extended culture (to the blastocyst stage), embryos are transferred at the 4-8-cell stage (end of Day 2) to HECM-9aa medium supplemented with 5% FBS (HyClone, v/v). Embryos were transferred to fresh HECM-9aa+5% FBS every other day. Harvested oocytes were examined under the microscope and separated on MI and MII. MI oocytes were allowed to mature to the MII stage for additional 3-4 hours by culturing in HECM-9aa media. Maturation was controlled by visual examination of cultures. Once the oocytes reached the MII stage, they were further used for artificial activation.

8. Hyaluronidase (Sigma H-3506) stock: for 10× stock, 50 mg was reconstituted in 10 ml of Hepes-buffered TALP medium, separated into 0.5 ml aliquots, and stored at ±20° C.

9. Light paraffin oil (Zander IVF; Vero Beach, Fla.)

10. Cell strainers (70 μm Nylon; Falcon; BD Biosciences; Bedford, Mass.)

11. Portable incubator (Minitube; Madison, Miss.)

12. Ultrasonography equipment (OOWYCR, Philips)

13. Dissecting microscope (SZ-61, Olympus America, Inc.)

### Methods

Protocols for controlled ovarian stimulation in Rhesus monkeys with recombinant human gonadotropins have been developed at the Oregon National Primate Research Center using the following steps:

1. Cycling females were monitored for menstruation and 1-4 days following onset, and were administered twice daily i.m injections of 30 IU recombinant human FSH (at 8 AM and 4 PM) for 8 days.

2. Females were administered Antide at a dose of 0.5 mg/kg, s.c. once a day for 8 days to suppress pituitary function and prevent spontaneous LH surges.

3. On the last two days of stimulation (days 7 and 8), females were additionally administered twice daily injections of recombinant human LH (30 IU i.m.).

4. On day 8, animals were anesthetized with ketamine (10 mg/kg body weight, i.m), and ovarian morphology was examined by ultrasonography. Typically, a responsive ovary was enlarged from 6 mm to an average diameter of 10 mm or greater and contained at least 5 large follicles, 2-4 mm in diameter.

5. On the morning of day 9, monkeys meeting these criteria were injected with recombinant hCG (1000 IU, i.m.) to induce oocyte maturation. Ovarian oocytes, which arrest at prophase I (GV), resumed meiosis in response to hCG and arrested again at metaphase II (MII). Approximately 20% of gonadotropin-treated females were discontinued at this time due to lack of adequate response as judged by ultrasonography. The percentage of “non-responders” varied by season, with an increase during the summer months, reaching over 35% in June and July. During summer, despite housing in controlled, constant environments, many females also became anovulatory, and it was impractical to attempt controlled ovarian stimulation. Females could be recycled for controlled ovarian stimulation, however, the response to recombinant human gonadotropins was gradually decreased with increasing numbers of stimulations, apparently due to an immune reaction. Practically, up to three stimulations on average could be performed per female with the recovery of a reasonable number of high quality oocytes. The availability of monkey recombinant gonadotropins would allow the more efficient and extended use of females.

### Laparoscopic Oocyte Recovery

Oocytes were collected by laporascopic follicular aspiration 27-33 hours after hCG injection via transabdominal needle aspiration of gravid ovarian follicles. Laparoscopy played a prominent role in the IVF laboratory, with most surgical procedures accomplished by the following steps:

1. Monkeys were anesthetized with isoflurane gas vaporized in 100% oxygen. Comprehensive physiologic monitoring of animals was conducted throughout the surgery, including ECG, peripheral oxygen saturation, and end-expired carbon dioxide. Orotracheal intubation and mechanical ventilation to maintain expired CO₂ at less than 50 mm Hg was mandatory.

2. Serile skin preparation and draping were performed after which the abdomen was insufflated with CO₂ at 15 mm Hg pressure. The viewing telescope was inserted via a small supraumbilical incision, with accessory ports placed in the paralumbar region.

3. The monkey were positioned in Trendeleburg, allowing the viscera to migrate in a cephalad direction exposing the reproductive organs.

4. A single small grasping forceps was used to stabilize the ovary for examination and needle aspiration. Rarely was a second accessory port and grasping forceps required for the experienced laparoscopist to perform this procedure.

5. After mobilization of the ovary, a 22 g hypodermic needle was connected to a source of continuous vacuum (−120 mm Hg), and inserted into individual follicles until all were aspirated.

6. Insufflation was reduced, and the incisions were closed with interrupted absorbable suture in an intradermal pattern.

7. Tubes containing follicular aspirates were placed into a portable incubator (Minitube) at 37° C. and transported quickly to the lab. The time between aspiration and oocyte recovery was minimized to avoid the detrimental effects of blood exposure, which usually contaminates the aspirates. The conventional approach of diluting aspirates with medium and searching for oocytes under dissecting a microscope was labor intensive often requiring 2-3 technicians. If desired, the recovery time can be minimized by sifting the aspirates through cell strainers.

8. A 10× hyaluronidase stock solution was added directly to the tubes containing aspirates at 1:10 ratio and incubated at 37° C. for 30 seconds.

9. The contents were gently agitated with a serological pipette to disaggregate cumulus and granulosa masses, and the entire aspirate was poured onto a cell strainer.

10. Oocytes were retained in the mesh, while blood, cumulus, and granulosa cells were sifted through the filter.

11. The strainer was quickly backwashed with TH3 medium, and the medium containing oocytes was collected in a Petri dish.

12. Oocytes were rinsed and were then easily identified in TH3 medium.

13. Any remaining cumulus cells were removed by manual clean up with a small bore pipette (approximately 125 □m in inner diameter).

14. Oocytes were observed at higher magnification for determination of their developmental stage (GV, MI or MII) as well as quality (granularity, shape, and color of the cytoplasm). On average, 40 oocytes were collected per stimulation, with over 70% matured or maturing (MII and MI stages).

15. After evaluation, oocytes were transferred into chemically defined, protein-free HECM-9aa medium at 37° C. in 5% CO₂, until further use. Most MI stage oocytes matured to the MII stage within 3-4 hours.

### Parthenogentic Oocyte Activation and Embryo Culture

Methods for activating oocytes have been described (Mitalipov, S. M., K. D. Nusser, et al. (2001). “Parthenogenetic activation of rhesus monkey oocytes and reconstructed embryos.” Biol Reprod 65(1): 253-9); (Mitalipov, S. M. et al., Biol Reprod 65, 253 (July, 2001); Susko-Parrish, J. L. et. al. Dev Biol 166, 729 (December, 1994)).

For the present study, mature metaphase II stage oocytes were activated by exposure to 5 μM ionomycin (CalBiochem, San Diego, Calif.) for 5 minutes in TALP/HEPES medium supplemented with 1 mg/ml BSA and then transferred for 5 minutes in TALP/HEPES medium supplemented with 30 mg/ml BSA and 2 mM 6-dimethylaminopurine followed by a 5-hour incubation in HECM-9 medium containing 2 mM 6-dimethylaminopurine at 37° C. in 5% CO₂, 5% O₂, and 90% N₂.

Alternatively, after ionomycin treatment, oocytes were incubated for 5 hours in HECM-9 medium containing 50 μM roscovitine (CalBiochem) and 5 μg/ml cytochalasin B or 7.5 μg/ml cycloheximide and 5 μg/ml cytochalasin B.

After activation, oocytes were placed in 4-well dishes (Nalge Nunc International Co., Naperville, Ill.) containing HECM-9 medium and cultured at 37° C. in 5% CO₂, 5% O₂, and 90% N₂. Embryos at the 8-16 cell stage were transferred to fresh plates of HECM-9 medium supplemented with 5% fetal bovine serum (FBS, Hyclone, Logan, Utah) and cultured for a maximum of 7 days with medium change every other day.

### PESC Isolation and Propagation

Zonae pellucidae of expanded blastocysts were removed with brief protease (0.5%) treatment, and inner cell mass cells (ICMs) were isolated using immunosurgery (Mitalipov, S., H. C. Kuo, et al. (2006). “Isolation and Characterization of Novel Rhesus Monkey Embryonic Stem Cell Lines.” Stem Cells, 24, 2177-86). ICMs were plated onto Nunc 4-well dishes containing mitotically-inactivated mEFs and ESC culture medium consisting of DMEM/F12 medium supplemented with 1% nonessential amino acids, 2 mM L-glutamine, 0.1 mM β-mercaptoethanol (all from Invitrogen, Carlsbad, Calif.) and 15% FBS. ICMs that attached to the feeder layer and initiated outgrowth were manually dissociated into small cell clumps with a microscalpel and replated onto new mEFs. After the first passage, colonies with ESC-like morphology were selected for further propagation, characterization, and low temperature storage. Medium was changed daily and ESC colonies were split every 5-7 days by manual disaggregation and replating collected cells onto dishes with fresh feeder layers. Cultures were maintained at 37° C., 3% CO₂, 5% O₂, and 92% N₂.

Following artificial activation of mature, metaphase II (MII) arrested oocytes, 38% of cleaved parthenotes developed to the expanded blastocyst-like stage with an efficiency statistically similar to sperm-fertilized embryos (Table 1). In Table 1, the percentages of 8-cell, morulae, and blastocysts are calculated based on the number of cleaved embryos. As indicated by footnote “a,” treatments with similar superscripts within a column are not significantly different (P>0.05).

TABLE 1
In vitro development of monkey sperm-fertilized embryos and
parthenotes

| | | | | | | Blastocyst | |
|---|---|---|---|---|---|---|---|
| | | | | | | used for | ESC lines |
| | | Cleaved | 8-cell | Morula | Blastocyst | ESC | isolated |
| Embryo origin | N | (%) | (%) | (%) | (%) | isolation | (%) |
| Conventional | 71 | 50 (70) | 47 (94) | 38 (76) | 27 (54)^{a} | 0 | N/A |
| IVF | | | | | | | |
| Intracytoplasmic | 40 | 38 (95) | 32 (84) | 29 (76) | 19 (50)^{a} | 0 | N/A |
| sperm injection | | | | | | | |
| Parthenotes | 104 | 100 (96) | 96 (96) | 93 (93) | 38 (38)^{a} | 17 | 5 (29%) |

### Cytogenetic Analysis

To karyotype PESCs, mitotically active PESCs in log phase were incubated with 120 ng/mL ethidium bromide for 40 minutes at 37° C. and 5% CO₂, followed by 120 ng/ml colcemid treatment for 20-40 minutes. Cells were dislodged with 0.25% trypsin, and centrifuged at 200×g for 8 minutes. The cell pellet was gently resuspended in 0.075 M KCl solution and incubated for 20 minutes at 37° C. followed by fixation with methanol:glacial acetic acid (3:1) solution. Fixed cells were dropped on wet slides, air dried, and baked at 90° C. for 1 hour. G-banding was performed using trypsin-EDTA and Lieschman Stain (GTL) by immersing slides in 1× trypsin—EDTA with two drops of 0.4M Na₂HPO₄ for 20 to 30 seconds. Slides were rinsed in distilled water and stained with Lieschman Stain for 1.5 minutes, rinsed in distilled water again, and air dried. For GTL-banding analysis, 20 metaphases were fully karyotyped under an Olympus BX40 microscope equipped with 10× and 100× plan-apo objectives. Images were then captured and cells karyotyped using a CytoVision® digital imaging system.

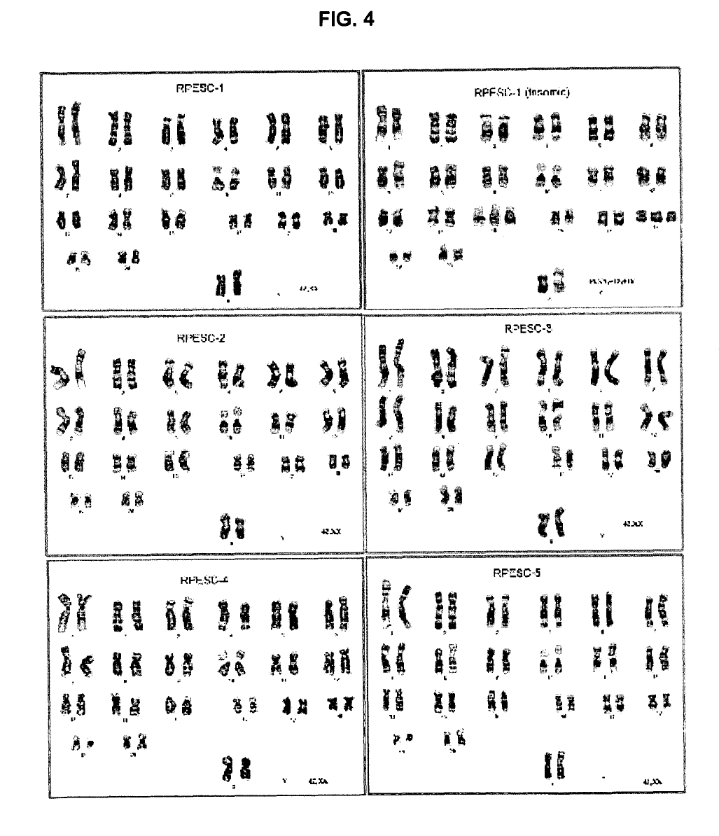

The parthenotes carry a diploid set of chromosomes (42XX) since second polar body extrusion is inhibited. Five karyotypically normal female PESC lines were derived from 17 blastocyst-like parthenotes for an efficiency of 30%, comparable to the corresponding yield with fertilized, bi-parental ESCs (FIG. 4) (Mitalipov, S., H. C. Kuo, et al. “Isolation and Characterization of Novel Rhesus Monkey Embryonic Stem Cell Lines.” Stem Cells, 24, 2177-86, 2006).

### Immunocytochemical Analysis

To examine the morphology of PESCs, the cells were plated onto glass culture (chamber) slides pre-coated with gelatin or polyornithine/laminin before fixation in 4% paraformaldehyde. After rinsing three times with PBS, ESCs were permeabilized with 0.2% Triton X-100 and 0.1% Tween-20 in PBS for 40 minutes at room temperature. Cells were then incubated with 2% normal serum for 30 minutes at room temperature, and after extensive washing, incubated with primary antibodies (OCT4, SSEA-3, SSEA-4, TRA-1-60, and TRA-1-81, all from Santa Cruz Biotechnology, Inc., Santa Cruz, Calif.) diluted to the optimal concentration (usually 1:200) with 0.05% Tween-20 for 40 minutes at room temperature. After rinsing as described above, cells were incubated with fluorophore-tagged second antibodies (Jackson ImmunoResearch Laboratories, Inc., West Grove, Pa.) diluted in the same solution as primary antibodies) for 40 minutes in the dark at room temperature followed by washing and counterstaining with DAPI for 10 minutes. The slides were covered with coverslips and examined under epifluorescence or confocal microscopy.

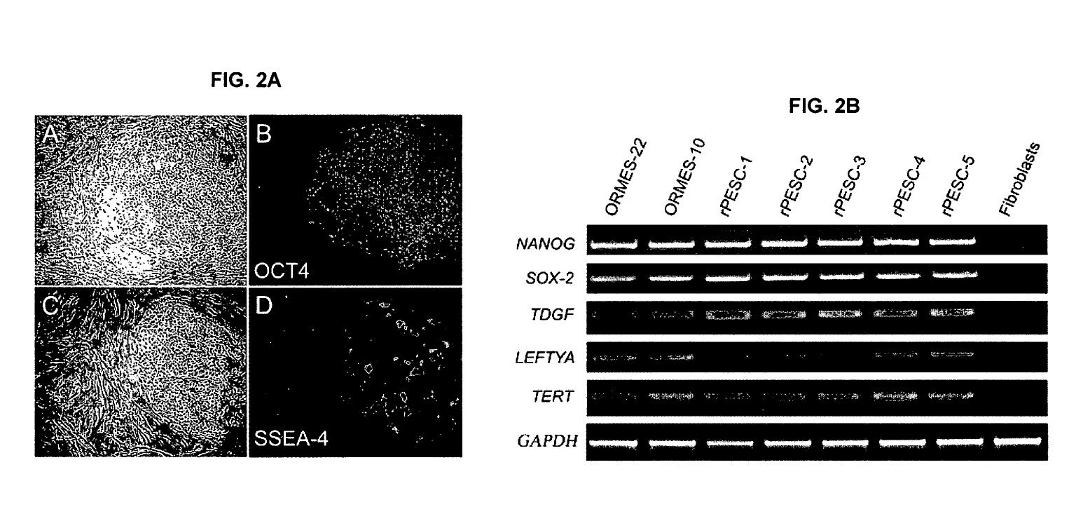

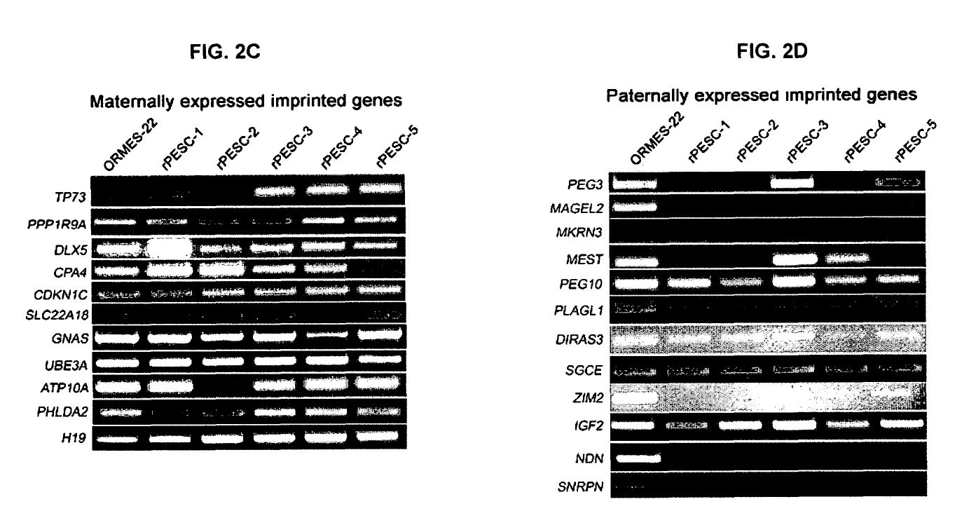

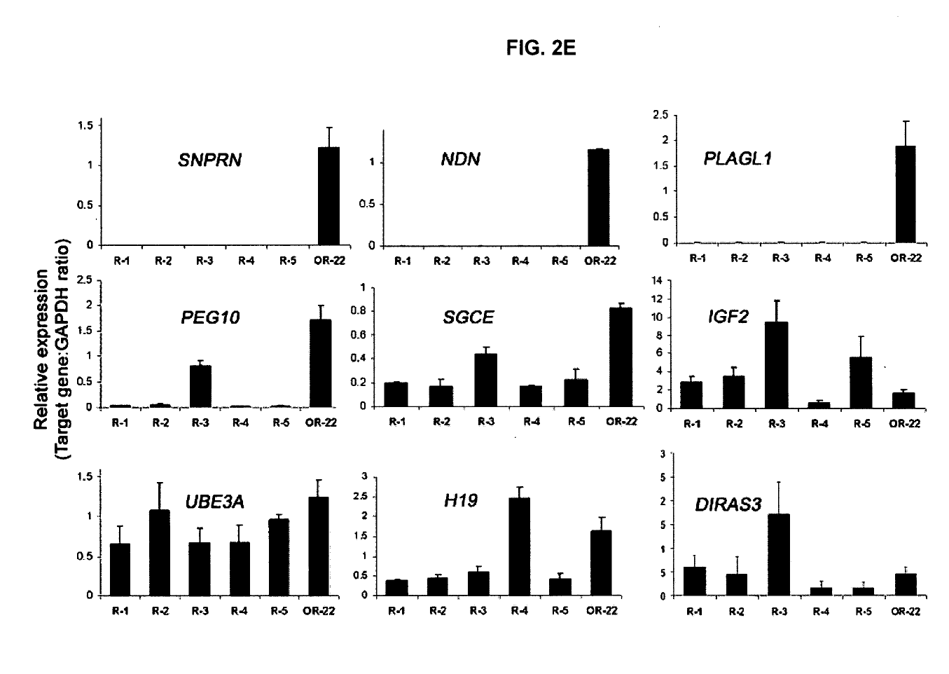

PESC lines were morphologically indistinguishable from bi-parental ESC controls (ORMES lines; Mitalipov, S. M. et al., Stem Cells (Jun. 1, 2006)). For example, PESCs expressed key pluripotent markers including OCT4, SSEA-3, SSEA-4, TRA-1-60, and TRA-1-81 based on immunohistochemical detection and expressed transcripts of several genes characteristic of pluripotent cells including NANOG, SOX-2, TDGF, LEFTYA, and TERT (FIGS. 2A-2B).

### Differentiation Potential

### In Vivo Differentiation of PESCs

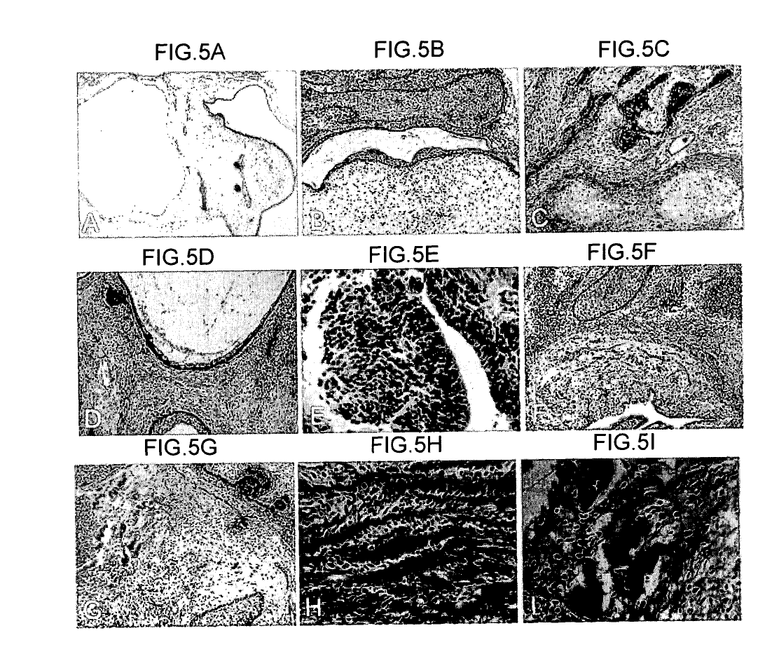

The absence of paternally expressed, imprinted genes in parthenotes may result in limited differentiation potential based on evidence from the mouse where ESCs reportedly did not contribute efficiently to mesodermal lineages, particularly to muscle tissues in chimeric animals and teratomas. The primate PESC lines described herein gave rise to cell lineages representative of all three embryonic germ layers upon injection into SCID mice. All PESC lines efficiently generated teratomas whose histological composition was indistinguishable from the bi-parental controls (FIGS. 5A-5I) containing both mixed cystic and solid regions. The cysts were lined by a simple low cuboidal epithelium and columnar type epithelium. The solid areas contained an admixture of mostly mature tissues derived from all three germ layers scattered haphazardly throughout the tumors. Ectoderm was represented by stratified squamous epithelium, neural tissue, and ganglion cells; mesodermal derivatives included cartilage, bone, muscle and fibrous connective tissue; and endodermal tissue was mostly represented by enteric-type columnar epithelium. Immature neuroepithelium arranged in tubules and rosettes with few mitoses and blastema-like tissue was also found in teratomas derived from both PESC lines and ORMES bi-parental controls.

### In Vitro Differentiation of PESCs

Mesodermal differentiation of PESC lines was induced in vitro by embryoid body production in suspension culture for 5-7 days followed by plating onto collagen-coated dishes for adherent culture. In particular, for embryoid body formation, entire or partially dissociated PESC colonies were loosely detached from feeder cells and transferred into 6 well, Ultra Low adhesion plates (Costar, Corning Incorporated, Acton, Mass.) for suspension culture. The cardiomyocyte protocol involved transfer of EBs onto collagen-coated culture dishes. Following attachment, embryoid bodies spontaneously developed into clusters of contracting cardiomyocytes. For studies on teratoma formation, 3-5 million undifferentiated ESCs from each cell line were harvested and injected into the hind leg muscle or subcutaneously into 4-week old, SCID, beige male mice using an 18 g needle. Four to five weeks after injection, mice were sacrificed and teratomas were dissected, sectioned, and histologically characterized for the presence of representative tissues of all three germ layers.

Approximately 7-14 days after plating, attachment, and further differentiation, spontaneously contracting cell aggregates were observed in all PESC lines. Analysis of these aggregates by RT-PCR revealed expression of markers specific for cardiomyocytes and muscle tissue (Table 2). Thus, diploid primate PESC lines can efficiently give rise to a wide variety of cell types and tissues representing all three germ layers.

### RT-PCR

To analyze these aggregates by RT-PCR, total RNA was extracted from PESCs and ESC-derived differentiated phenotypes using a RNA purification kit (Invitrogen, City, State) according to the manufacturer's instructions. Total RNA was treated with DNase I before cDNA preparation using Super-Script III First-Strand Synthesis System for reverse transcription-polymerase chain reaction (RT-PCR) (Invitrogen). The first strand cDNA was further amplified by PCR using individual primer pairs for specific genes. The sequences and annealing temperature of each primer pair is listed in Table 3. All PCR samples were analyzed by electrophoresis on 1.6% agarose gel containing 0.5 ug/ml ethidium bromide. Predicted sequences were confirmed by purifying RT-PCR products via QIAquick Gel Extraction Kit (QIAGEN Inc., Valencia, Calif.), and sequenced in both directions at the OHSU Vollum Sequencing Core on a Genetic Analyzer 3100 (Applied Biosystems, Foster City, Calif.) using amplification primers listed in Table 3. Methylation analysis of the IGF2/H19 IC by Southern-blot analysis was carried out as described previously (Mitalipov, S., L. Clepper, et al. (2007). “Methylation Status of Imprinting centers for H19/IGF2 and SNURF/SNRPN in Primate Embryonic Stem Cells.” Stem Cells 25(3): 581-8.).

Analysis of these aggregates by RT-PCR revealed expression of markers specific for cardiomyocytes and muscle tissue (Table 2). These results indicate that diploid primate PESC lines can efficiently give rise to a wide variety of cells and tissues representing all three germ layers. Table 2 indicates the presence (+) or absence (−) of PCR cDNA products generated from probes listed in Table 3 for each gene.

TABLE 2
RT-PCR expression analysis of cardiac-specific genes in contracting
aggregates derived from PESCs and a bi-parental control (ORMES-22)

| | Adult | ORMES- | ORMES-22 | RPESC-2 | RPESC-3 | RPESC-4 |
|---|---|---|---|---|---|---|
| | heart | 22 | cardiac | cardiac | cardiac | cardiac |
| Gene | tissue | undifferentiated | differentiation | differentiation | differentiation | differentiation |
| GAPDH | + | + | + | + | + | + |
| ALPHAMHC | + | − | + | + | + | − |
| hANP | + | − | + | + | + | + |
| NKX2.5 | + | − | + | + | + | + |
| TROPONIN T | + | − | + | + | + | + |
| MLC-2A | + | − | − | + | + | − |

TABLE 3
Primer sequences, product size, and Tm for RT-PCRs and qPCRs.

| | | | | |
|---|---|---|---|---|
| GENE | 5′ PRIMER | 3′ PRIMER | Bp | Tm |

IMPRINTED GENES

| | | | | |
|---|---|---|---|---|
| TP73 | ACTTCGAGGTCACTTTCCAGCAGT | TATTGCCTTCCACACGGATGAGGT | 285 | 60 |
| | (SEQ ID NO: 1) | (SEQ ID NO: 36) | | |
| PPP1R9A | TAAGACCACCCTGCTGTGGAAACA | GTTTGCCAGTGTTGCTGAGATGCT | 317 | 60 |
| | (SEQ ID NO: 2) | (SEQ ID NO: 37) | | |
| DLX5 | ACCATCCTTCTCAGGAATCGCCAA | TGTGTTTGTGTCAATCCCAGCGAG | 457 | 60 |
| | (SEQ ID NO: 3) | (SEQ ID NO: 38) | | |
| CPA4 | TCTCTTTCCACCTTCAATCGGCCT | CAGGAAAGTCTGCGGCAATGTTGT | 259 | 56 |
| | (SEQ ID NO: 4) | (SEQ ID NO: 39) | | |
| CDKN1C | AGCACATCTACGATGGAGCGTCTT | AGTCGCTGTCCACTTCGGTCCACT | 253 | 60 |
| | (SEQ ID NO: 5) | (SEQ ID NO: 40) | | |
| SLC22A18 | GCCACTTCTCGGAGGAGGTGCT | GGAGCAGTGGTTGTACAGAGGCG | 231 | 55 |
| | (SEQ ID NO: 6) | (SEQ ID NO: 41) | | |
| GNAS | ATGGTGAGAAGGCAACCAAAGTGC | GAAGGTGCATGCGCTGAATGATGT | 912 | 60 |
| | (SEQ ID NO: 7) | (SEQ ID NO: 42) | | |
| UBE3A | ATGCACTTGTCCGGCTAGAGATGA | AGCCAGACCCAGTACTATGCCAAT | 263 | 60 |
| | (SEQ ID NO: 8) | (SEQ ID NO: 43) | | |
| ATP10A | AACCGGCTCAAGACCACCAAGTA | TGTAGTCCTCCCATAGGTCCCTGAA | 211 | 60 |
| | (SEQ ID NO: 9) | (SEQ ID NO: 44) | | |
| PHLDA2 | CTTCCAGCTATGGAAGAAGAAGCG | CGGCGGTTCTGGAAATCGATGA | 255 | 55 |
| | (SEQ ID NO: 10) | (SEQ ID NO: 45) | | |
| H19 | AGCTAGAGGAACCAGACCTCATCA | ATGGAATGCTTGAAGGTTGCCC | 525 | 62 |
| | (SEQ ID NO: 11) | (SEQ ID NO: 46) | | |
| PEG3 | TCGCTGAGGACAGGAAACCT | ACTCCCTTGCTCTTCCCGAT | 303 | 56 |
| | (SEQ ID NO: 12) | (SEQ ID NO: 47) | | |
| MAGEL2 | TAAAGAGCGCAGGACCTCCTCAAA | TGCCTTTGAGGCATTCATGTTGGG | 346 | 60 |
| | (SEQ ID NO: 13) | (SEQ ID NO: 48) | | |
| MKRN3 | GCGGCATTTGGACAAAGCAGATCA | ATCACAGGCAAGGAAAGGGAGGAT | 283 | 60 |
| | (SEQ ID NO: 14) | (SEQ ID NO: 49) | | |
| MEST | ATGGGATAACGCGGCCATGGTG | TTCCAACCACACCCACAGAGTCTT | 218 | 56 |
| | (SEQ ID NO: 15) | (SEQ ID NO: 50) | | |
| PEG10 | ACAACAACAACTCCAAGCACACCG | TCTGGGTTGCCATCGAACTTCTCT | 322 | 60 |
| | (SEQ ID NO: 16) | (SEQ ID NO: 51) | | |
| PLAGL1 | GCCACGTTTCCCTGCCAATTATGT | TCTCTTCCGCATGGGATTTGAGGT | 433 | 60 |
| | (SEQ ID NO: 17) | (SEQ ID NO: 52) | | |
| DIRAS3 | TCCCAACGATGGGTAACGTCAGTT | TCCAGGGTTTCCTTCTTGGTGACT | 382 | 60 |
| | (SEQ ID NO: 18) | (SEQ ID NO: 53) | | |
| SGCE | TACCCATCAGCAGGTGTCCTCTTT | AGGTGCGCCTATTGTAGGCAGTTA | 265 | 60 |
| | (SEQ ID NO: 19) | (SEQ ID NO: 54) | | |
| ZIM2 | TGTACCAACCGGAAGACGACAACA | TCGCCATCACAGGAAGGGAAAGAT | 228 | 60 |
| | (SEQ ID NO: 20) | (SEQ ID NO: 55) | | |
| IGF2 | CCAAAGTCCCGCTAAGATTCTCCA | GCAAAGATGATCCCTAGGTGTGCT | 471 | 65 |
| | (SEQ ID NO: 21) | (SEQ ID NO: 56) | | |
| NDN | GAGCCGCCCGAATACGAGTT | GCAGGAGCAGTCTACCCCAA | 564 | 60 |
| | (SEQ ID NO: 22) | (SEQ ID NO: 57) | | |
| SNPRN | AGTTACTGTGGATGAGGGTGATGC | CACCCAGGACCTTCCACTCATTTA | 474 | 58 |
| | (SEQ ID NO: 23) | (SEQ ID NO: 58) | | |

STEMNESS GENES

| | | | | |
|---|---|---|---|---|
| OCT4 | TGGAGAAGGAGAAGCTGGAGCAAA | GAAGCTAAGCTGCAGAGCCTCAAA | 212 | 60 |
| | (SEQ ID NO: 24) | (SEQ ID NO: 59) | | |
| NANOG | CAGAACTGTGTTCTCTTCCACCCA | CGCTGATTAGGCTCCAACCATACT | 801 | 60 |
| | (SEQ ID NO: 25) | (SEQ ID NO: 60) | | |
| TDGF | CATATTGGCCAGTCTGGTCTCGAA | ATTCCTTCAGCACTCTGGTTCCTC | 370 | 56 |
| | (SEQ ID NO: 26) | (SEQ ID NO: 61) | | |
| TERT | AGCTATGCCCGGACCTCCAT | GCCTGCAGCAGGAGGATCTT | 185 | 60 |
| | (SEQ ID NO: 27) | (SEQ ID NO: 62) | | |
| LEFTYA | CCTGTGTCCTTCCATTTCCTGTCT | AAGCCCTTCATCCTTCCTCTTAGC | 366 | 60 |
| | (SEQ ID NO: 28) | (SEQ ID NO: 63) | | |
| SOX2 | CCCCCGGCGGCAATAGCA | TCGGCGCCGGGGAGATACAT | 448 | 60 |
| | (SEQ ID NO: 29) | (SEQ ID NO: 64) | | |

CARDIAC GENES

| | | | | |
|---|---|---|---|---|
| GAPDH | GTGGTCTCCTCCGACTTCAACA | GTCTCTCTCTTCCTCTTGTGCTCT | 217 | 61 |
| | (SEQ ID NO: 30) | (SEQ ID NO: 65) | | |
| Alpha-MHC | GTCATTGCTGAAACCGAGAATG | GCAAAGTACTGGATGACACGCT | 413 | 61 |
| | (SEQ ID NO: 31) | (SEQ ID NO: 66) | | |
| hANP | GAACCAGAGGGGAGAGACAGAG | CCCTCAGCTTGCTTTTTAGGAG | 406 | 61 |
| | (SEQ ID NO: 32) | (SEQ ID NO: 67) | | |
| NKX2.5 | TGGCTACAGCTGCACTGCCG | GGATCCATGCAGCGTGGAC | 167 | 57 |
| | (SEQ ID NO: 33) | (SEQ ID NO: 68) | | |
| Mlc2a | ACAGAGTTTATTGAGGTGCCCC | AAGGTGAAGTGTCCCAGAGG | 381 | 61 |
| | (SEQ ID NO: 34) | (SEQ ID NO: 69) | | |
| cTnT | GGCAGCGGAAGAGGATGCTGAA | GAGGCACCAAGTTGGGCATGAACGA | 150 | 64 |
| | (SEQ ID NO: 35) | (SEQ ID NO: 70) | | |

qPCR

| | | | |
|---|---|---|---|
| GENE | 5′ PRIMER | 3′ PRIMER | PROBE |

| | | | |
|---|---|---|---|
| PLAG1 | AGTACAACACCATGCTGGGCTAT | TGCTGGCCGCATGGA | AGAGGCACCTGGCC |
| | (SEQ ID NO: 71) | (SEQ ID NO: 81) | (SEQ. ID NO: 91) |
| SGCE | ACCCAAAACCTGGCGAGAT | TCCAGGTCGGTCTGGGTAAC | AGTAATGATCCCATAA |
| | (SEQ ID NO: 72) | (SEQ ID NO: 82) | CATT |
| | | | (SEQ. ID NO: 92) |
| SNRPN | AAGCAACCAGAGCGTGAAGAA | TCCCCACGCAGCAACAC | AGCGGGTTTTGGGTCT |
| | (SEQ ID NO: 73) | (SEQ ID NO: 83) | (SEQ. ID NO: 93) |
| H19 | CCTCCCCGACTCTGTTTCC | CACAACTCCAACCAGTGCAAA | CCGTCCCTTCTGAATT |
| | (SEQ ID NO: 74) | (SEQ ID NO: 84) | (SEQ. ID NO: 94) |
| IGF2 | GTCGGCCCAGCCAGAGT | CGGCTACCATCATCTCCATTG | AGGAAGGAGTTTGGCC |
| | (SEQ ID NO: 75) | (SEQ ID NO: 85) | (SEQ. ID NO: 95) |
| NECDIN | TGTCTCCGAGGACTAGCCAAGT | GCCCTGGTGAGGATCAGAAA | TGGAGGCAGATGAAT |
| | (SEQ ID NO: 76) | (SEQ ID NO: 86) | (SEQ. ID NO: 96) |
| UBE3A | GAAGGAGAACAAGGAGTTGATGAAG | CCTCCACAACCAGCTGAAAAA | AGGTGTTTCCAAAGAA |
| | (SEQ ID NO: 77) | (SEQ ID NO: 87) | (SEQ. ID NO: 97) |
| PEG10 | CCCTTCGAGAGCAAGTGGAA | GCGGAGCTCGATGTCATCAT | CCACCCCTGAGGATG |
| | (SEQ ID NO: 78) | (SEQ ID NO: 88) | (SEQ. ID NO: 98) |
| GAPDH | GGTGGTCTCCTCCGACTTCA | ACCAGGAAATGAGCTTGACAAAG | CCCACTCTTCCACCTT |
| | (SEQ ID NO: 79) | (SEQ ID NO: 89 | CGACGCTG |
| | | | (SEQ. ID NO: 99) |
| OCT4 | CCCACTGGTGCCGTGAA | TTGGCAAATTGCTCGAGTTCT | GGACTCCTCCGGGTTT |
| | (SEQ ID NO: 80) | (SEQ ID NO: 90) | TGCTCCAG |
| | | | (SEQ. ID NO: 100) |

### Heterozygosity of Primate PESCs

Since PESC lines have diploid sets of chromosomes achieved by retention of the second polar body following artificial activation, each homologous chromosome pair is a couplet of sister chomatids expected to display high levels of homozygosity in daughter cells. However, we hypothesized that due to homologous chromosome crossing-over and recombination during meiosis, heterozygosity in primate PESCs could occur. The heterozygosity of primate PESCs was determined as follows using microsatellite/short tandem repeat (STR) analysis on PESCs.

### STR Analysis

For STR genotyping, DNA was extracted from blood or cultured cells using commercial kits (Gentra, Minneapolis, Minn.). Six multiplexed PCR reactions were set up for the amplification of 41 markers representing 25 autosomal loci, 1 X-linked marker (DXS22685), and 15 autosomal, MHC-linked loci. Based on the published Rhesus monkey linkage map (Rogers, J., R. Garcia, et al. (2006). “An initial genetic linkage map of the rhesus macaque (Macaca mulatta) genome using human microsatellite loci.” Genomics 87(1): 30-8.), these markers are distributed in about 19 chromosomes. Two of the markers included in the panel, MFGT21 and MFGT22 (Domingo-Roura, X., T. Lopez-Giraldez, et al. (1997). “Hypervariable microsatellite loci in the Japanese macaque (Macaca fuscata) conserved in related species.” Am J Primatol 43(4): 357-60), were developed from Macaca fuscata and do not have a chromosome assignment. PCRs were set up in 25 μl reactions containing 30-60 ng DNA, 2.5 mM MgCl₂, 200 μM dNTPs, 1×PCR buffer II, 0.5 U Amplitaq (Applied Biosystems), and fluorescence-labeled primers in concentrations ranging from 0.06 to 0.9 μM, as required for each multiplex PCR. Cycling conditions consisted of 4 cycles of 1 minute at 94° C., 30 seconds at 58° C., 30 seconds at 72° C., followed by 25 cycles of 45 seconds at 94° C., 30 seconds at 58° C., 30 seconds at 72° C. and a final extension at 72° C. for 30 minutes. PCR products were separated by capillary electrophoresis on ABI 3730 DNA Analyzer (Applied Biosystems) according to the manufacturer's instructions. Fragment size analysis and genotyping was done with the computer software STR and (available at the world wide web at “vgl.ucdavis.edu/informatics/Strand/”). Primer sequences for MEW-linked STRs 9P06, 246K06, 162B17(A and B), 151L13, 268P23, and 222I18 were designed from the corresponding Rhesus monkey BAC clone sequences deposited in GenBank (accession numbers AC148662, AC148696, AC148683, AC148682, AC148698, and AC148689, respectively). Loci identified by letter “D” prefix were amplified using heterologous human primers.

The results of microsatellite/short tandem repeat (STR) analysis were compared to those of gDNA isolated from the peripheral blood of oocyte donors. The analysis of 29 STR loci confirmed that RPESC-1, -2, and -3 were derived from the same oocyte donor and that RPESC-4 and -5 were from two other unrelated females (Table 4)

TABLE 4
STR genotypes in monkey oocyte donors and PESC lines (heterozygous
loci are highlighted in gray)

| | Egg | | | | | | | |
|---|---|---|---|---|---|---|---|---|
| | donor | | | | Egg donor | | Egg donor | |
| Locus | #22184 | RPESC-1 | RPESC-2 | RPESC-3 | #16715 | RPESC-4 | #18178 | RPESC-5 |
| D1S548 | 190/206 | 206/206 | 190/206 | 190/190 | 190/206 | 190/206 | 199/202 | 199/202 |
| D2S1333 | 277/297 | 277/297 | 277/297 | 277/297 | 289/293 | 289/289 | 277/277 | 277/277 |
| D3S1768 | 205/221 | 205/221 | 205/221 | 205/221 | 201/217 | 201/217 | 215/273 | 215/273 |
| D4S2365 | 283/287 | 283/287 | 283/287 | 287/287 | 279/283 | 283/283 | 273/281 | 273/281 |
| D4S413 | 131/131 | 131/131 | 131/131 | 131/131 | 129/131 | 129/129 | 129/141 | 129/141 |
| D5S1457 | 132/132 | 132/132 | 132/132 | 132/132 | 132/136 | 136/136 | 128/136 | 128/128 |
| D6S276 | 215/225 | 215/225 | 225/225 | 215/225 | 211/225 | 211/225 | 239/241 | 239/241 |
| D6S291 | 206/208 | 206/208 | 206/206 | 208/208 | 208/208 | 208/208 | 205/217 | 205/217 |
| D6S501 | 180/184 | 180/180 | 180/184 | 180/184 | 180/180 | 180/180 | 176/180 | 176/180 |
| D6S1691 | 197/197 | 197/197 | 197/197 | 197/197 | 203/203 | 203/203 | 175/195 | 175/195 |
| D7S513 | 209/239 | 209/239 | 209/239 | 209/239 | 189/199 | 199/199 | 190/203 | 190/203 |
| D7S794 | 124/132 | 124/132 | 124/132 | 124/132 | 128/128 | 128/128 | 127/127 | 127/127 |
| D8S1106 | 148/160 | 148/160 | 148/160 | 148/160 | 144/156 | 144/156 | 140/152 | 140/152 |
| D9S921 | 187/195 | 187/195 | 187/195 | 187/195 | 179/199 | 179/199 | 187/195 | 187/195 |
| D10S1412 | 157/157 | 157/157 | 157/157 | 157/157 | 157/157 | 157/157 | 155/155 | 155/155 |
| D11S2002 | 252/252 | 252/252 | 252/252 | 252/252 | 252/260 | 260/260 | 260/260 | 260/260 |
| D11S925 | 308/330 | 308/330 | 308/330 | 308/308 | 308/312 | 308/312 | 305/327 | 305/327 |
| D12S364 | 290/290 | 290/290 | 290/290 | 290/290 | 280/290 | 280/290 | 286/288 | 286/288 |
| D12S67 | 117/212 | 117/212 | 117/212 | 117/212 | 113/121 | 113/113 | 125/188 | 188/188 |
| D13S765 | 224/232 | 224/232 | 232/232 | 224/232 | 224/228 | 224/228 | 222/222 | 222/222 |
| D15S823 | 333/357 | 357/357 | 333/333 | 333/333 | 353/353 | 353/353 | 369/369 | 369/369 |
| D16S403 | 162/164 | 162/164 | 162/164 | 162/164 | 152/156 | 156/156 | 163/165 | 163/163 |
| D17S1300 | 248/260 | 248/260 | 248/260 | 248/260 | 232/248 | 232/232 | 255/255 | 255/255 |
| D18S537 | 162/174 | 162/174 | 162/174 | 162/162 | 174/174 | 174/174 | 166/174 | 166/174 |
| D18S72 | 308/322 | 308/308 | 308/322 | 322/322 | 308/308 | 308/308 | 312/312 | 312/312 |
| D22S685 | 319/319 | 319/319 | 319/319 | 319/319 | 315/323 | 315/323 | 315/319 | 315/315 |
| DXS2506 | 262/262 | 262/262 | 262/262 | 262/262 | 262/262 | 262/262 | 262/274 | 262/274 |
| MFGT21 | 111/115 | 111/115 | 111/115 | 111/115 | 119/119 | 119/119 | 108/118 | 108/118 |
| MFGT22 | 110/120 | 110/110 | 110/120 | 110/110 | 104/104 | 104/104 | 92/100 | 92/100 |

The oocyte donor for RPESC-1, -2, and -3 (female #22184) exhibited 21 heterozygous loci out of the 29 analyzed. RPESC-1, -2, and -3 showed the presence of both alleles at the majority of these loci; 16, 17, and 13 heterozygous loci out of 21, respectively (Table 4 in gray). Similarly, RPESC-4 inherited 9 heterozygous loci out of 18 present in the oocyte donor, and RPESC-5 exhibited 16 out of 20 heterozygous STRs. In order to eliminate the possibility that Rhesus monkey PESCs were unique, polymorphic loci in the cynomolgus monkey PESC line, Cyno-1, and the oocyte donor female were examined (Cibelli, J. B. et al., Science 295, 819 (Feb. 1, 2002)). This line displayed 13 heterozygous loci out of 24 informative STRs present in the oocyte donor female. These results indicate that primate PESC lines display significant levels of genetic heterozygosity due, presumably, to recombination events occurring during homologous chromosome crossing-over at the first meiotic division.

### SNP Analysis

To corroborate these results, a panel of 60 single nucleotide polymorphisms (SNPs) localized to the 3′ ends of specific Rhesus monkey genes were monitored (Ferguson, B. et al., BMC Genomics 8, 43 (Feb. 7, 2007)). SNP genotyping was performed using iPLEX reagents and protocols for multiplex PCR, single base primer extension (SBE), and the generation of mass spectra according to the manufacturer's instructions (iPLEX Application Note, Sequenom, San Diego, Calif.). Four multiplexed assays containing 28, 17, 8, and 7 SNPs were included. Initial multiplexed PCRs were performed in 5 μl reactions on 384-well plates containing 5 ng of genomic DNA. Reactions contained 0.5 U HotStar Taq polymerase (QIAGEN), 100 nM primers, 1.25× HotStar Taq buffer, 1.625 mM MgCl₂, and 500 μM dNTPs. Following enzyme activation at 94° C. for 15 minutes, DNA was amplified with 45 cycles of 94° C. for 20 seconds, 56° C. for 30 seconds, and 72° C. for 1 minute, followed by a 3 minute extension at 72° C. Unincorporated dNTPs were removed using shrimp alkaline phosphatase (0.3 U, Sequenom). Single-base extension was carried out by the addition of SBE primers at concentrations from 0.625 μM to 1.25 using iPLEX enzyme and buffers (Sequenom). SBE products were measured using the MassARRAY Compact system, and mass spectra were analyzed using TYPER software (Sequenom).

Of the 13 informative SNPs detected in oocyte donor #22184, her RPESC lines-1, -2, and -3 inherited both alleles at 8, 8, and 7 loci, respectively (Table 5 in gray). RPESC-4 displayed 10 heterozygous loci out of 18 present in the oocyte donor (# 16715), and RPESC-5 exhibited 10 SNPs out of 16 existing in the donor (#18178). These results are consistent with the existence of high levels of heterozygosity in monkey PESC lines. Based on an analysis of 28 heterozygous microsatellite loci, and 32 informative SNPs present in the oocyte donors, diploid monkey PESCs restored heterozygosity at 64% of loci.

TABLE 5
SNP analysis of monkey oocyte donors and PESC lines

| | | | | | | | | |
|---|---|---|---|---|---|---|---|---|
| SNPs | #22184 | RPESC-1 | RPESC-2 | RPESC-3 | #16715 | RPESC-4 | #18178 | RPESC-5 |

| | | | | | | | | |
|---|---|---|---|---|---|---|---|---|
| BCHE: 76 | C | C | C | C | GC | G | C | C |
| BCHE: 447 | AG | G | G | G | AG | A | AG | A |
| CCL8: 516 | G | G | G | G | AG | A | AG | G |
| CCR1: 641 | G | G | G | G | G | G | G | G |
| CCR9: 315 | C | C | C | C | C | C | CT | CT |
| CD40LG: 748 | G | G | G | G | G | G | AG | AG |
| CD44: 471 | T | T | T | T | T | T | T | T |
| CD69: 294 | CT | CT | CT | CT | CT | CT | C | C |
| CD74: 213 | C | C | C | C | C | C | CT | CT |
| CD74: 344 | CT | CT | CT | CT | CT | CT | C | C |
| CFTR: 796 | AG | G | G | A | G | G | AG | AG |
| CX3CR1: 593 | AG | AG | AG | AG | AG | AG | G | G |
| CXCL12: 173 | C | C | C | C | T | T | CT | C |
| CYP11A1: 150 | G | G | G | G | AG | G | AG | G |
| FAS: 135 | A | A | A | A | AG | A | A | A |
| FSHR: 784 | CG | CG | CG | C | CG | CG | C | C |
| HTATSF1: 636 | C | C | C | C | C | C | CT | CT |
| IL1: 755 | AT | AT | AT | AT | AT | AT | AT | AT |
| IL2RA: 124 | C | C | C | C | CT | CT | C | C |
| INHBB: 131 | C | C | C | C | TC | TC | C | C |
| ITGA: 321 | A | A | A | A | A | A | AG | AG |
| LRP8: 647 | T | T | T | T | C | C | CT | CT |
| NDN: 135 | G | G | G | G | AG | AG | G | G |
| NOS1: 216 | AG | AG | AG | AG | AG | AG | ND | ND |
| NR4C1: 458 | T | T | T | T | T | T | AT | AT |
| PYY: 151 | T | T | T | T | TC | TC | T | T |
| SIRT1: 277 | GT | GT | GT | GT | G | G | G | G |
| SLC5A7: 79 | G | G | G | G | G | G | G | G |
| SLC6A4: 132 | GC | C | C | C | C | C | GC | G |
| SNCA: 394 | TC | TC | TC | C | C | C | ND | ND |
| STAR: 522 | GT | G | G | GT | GT | G | GT | T |
| TLR4: 735 | T | T | T | T | TC | C | T | T |
| TLR5: 389 | TC | C | C | C | TC | T | C | C |
| TNF: 82 | T | T | T | T | T | T | CT | CT |
| XCL1: 320 | C | C | C | C | T | T | T | T |

ND-not determined

### Imprinting in Primate PESCs

Paternally expressed genes (e.g., genes normally silenced by passage through the female germline) are expected to be absent in parthenotes, since all of the genetic material is of maternal origin. This expectation was challenged in undifferentiated PESC lines by conducting expression analysis of known imprinted genes, as described above. Bi-parental ORMES cell lines served as controls. As expected, transcripts of 11 maternally expressed genes were detected in both PESCs and bi-parental ESCs (Table 6, FIG. 2C). Expression levels of maternally expressed UBE3A in PESC lines were similar to that of bi-parental ORMES-22, while H19 gene activity varied among PESC lines (FIG. 2E). However, strikingly, amplified RT-PCR products of several imprinted genes normally expressed from the paternal allele, including PEG10, PLAGL1, DIRAS3, SGCE and IGF2, were also detected in PESC lines (FIG. 2D).

TABLE 6
Expression analysis of imprinted genes in monkey PESCs and bi-
parental (ORMES lines) ESCs

| | | ORMES- | ORMES- | | | | | |
|---|---|---|---|---|---|---|---|---|
| Gene | Muscle | 22 | 10 | RPESC-1 | RPESC-2 | RPESC-3 | RPESC-4 | RPESC-5 |

Maternally expressed

| | | | | | | | | |
|---|---|---|---|---|---|---|---|---|
| TP73 | + | + | + | + | + | + | + | + |
| PPP1R9A | + | + | + | + | + | + | + | + |
| DLX5 | + | + | + | + | + | + | + | + |
| CPA4 | + | + | + | + | + | + | + | + |
| CDKN1C | + | + | + | + | + | + | + | + |
| SLC22A18 | + | + | + | + | + | + | + | + |
| GNAS | + | + | + | + | + | + | + | + |
| UBE3A | + | + | + | + | + | + | + | + |
| ATP10A | + | + | + | + | + | + | + | + |
| PHLDA2 | −− | + | + | + | + | + | + | + |
| H19 | + | + | + | + | + | + | + | + |

Paternally expressed

| | | | | | | | | |
|---|---|---|---|---|---|---|---|---|
| PEG3 | + | + | + | −− | −− | + | −− | + |
| MAGEL2 | + | + | + | −− | −− | −− | −− | −− |
| MKRN3 | + | + | −− | −− | −− | −− | −− | −− |
| MEST | + | + | + | −− | −− | + | + | −− |
| PEG10 | + | + | + | + | + | + | + | + |
| PLAGL1 | + | + | + | + | + | + | + | + |
| DIRAS3 | + | + | + | + | + | + | + | + |
| SGCE | + | + | + | + | + | + | + | + |
| ZIM2 | + | + | + | −− | −− | + | + | + |
| IGF2 | + | + | + | + | + | + | + | + |
| NDN | + | + | + | −− | −− | −− | −− | −− |
| SNRPN | + | + | + | −− | −− | −− | −− | −− |

In Table 6, “+” indicates that the gene is expressed, and “−” indicates that the product encoded by the gene is absent.

Real-time PCR was used to examine imprinting at various loci. Quantitative real-time PCR (qPCR) was performed on total RNA samples from each PESC line and bi-parental ORMES-22. RNA was treated with RNase-free DNase (Invitrogen) to remove genomic DNA traces. The RNA concentrations were determined by Nanodrop ND 1000 spectrophotometer, and the quality was analyzed using an Agilent Labchip Bioanalyzer (Agilent Technologies). The cDNAs were synthesized from 800 ng of total RNA sample with SuperScript III reverse transcriptase (200 U/μl) (Invitrogen) using oligo(dT) primers. TaqMan probes and primers were designed using ABI Primer Express and probes were synthesized by Applied Biosystems (Foster City, Calif.). The real-time PCR primers were synthesized by Integrated DNA Technologies (Coralville, Iowa) (Table 3). qPCR was performed on an ABI 7500 Fast Real-time PCR System with the SDS 1.4.0 program and using the ABI TaqMan Fast Universal PCR master mix (Applied Biosystems). All reactions were carried out in triplicate. The final concentration of the real-time primers was 300 nM, and the final concentration of the real-time probes was 250 nM. Initially, the OCT4 qPCR results obtained for a 5-fold dilution series for ORMES-22 cDNAs were examined to determine the optimum primer dilution for future reactions. The control sample consisted of equal amounts of ORMES-22 cell line. With each subsequent run, duplicate 5-fold dilutions of GAPDH and each gene were included as standard curves. The cycling profile for each run was 95° C. for 20 seconds and 40 cycles of 95° C. for 3 seconds followed by 60° C. for 30 seconds using the default ramp rate. The relative gene expression for SNRPN, NDN, PLAGL1, PEG10, SGCE, IGF2, UBE3A and H19 was calculated using standard curve method followed by normalization with endogenous GAPDH.

Microarray analysis was conducted to investigate gobal expression profiles in PESCs. Total RNA was isolated from cell colonies selected for the appropriate ESC morphology (flat monolayer colony with distinctive cobbled stem cell morphology and a high nucleo-cytoplasmic ratio) using the Invitrogen TRIZOL® reagent, followed by further purification with the Qiagen RNeasy MinElute Cleanup Kit. For each cell line examined three biological replicates were used and each replicate represented either a different passage or different sub-line (which had been cultured independently and without mixing for several passages). The RNA samples were quantified using the NANODROP™ ND-1000 UV-Vis spectrophotometer (Nanoprop Technologies, Wilmington, Del.) and the quality of the RNA was assessed using Lab-on-a-Chip RNA Pico Chips and the 2100 Bioanalyzer (Agilent Technologies, Palo Alto, Calif.). Samples with electropherograms that showed a size distribution pattern predictive of acceptable microarray assay performance were considered to be of good quality. Two micrograms of total RNA from each sample were amplified and labeled using a single cycle cDNA synthesis and an in vitro transcription cRNA-RNA labeling system (GeneChip One-Cycle Target Labeling and Control Reagents; Affymetrix, Inc., Santa Clara, Calif.). Following successful cRNA amplification, 10 μg of labeled target cRNA was hybridized to Rhesus Macaque Genome Arrays (Affymetrix) using standard protocols, as described in the GeneChip Expression Analysis manual. The rhesus monkey array contains 52,865 probe sets, representing over 20,000 genes. The arrays were scanned using the GeneChip laser scanner (Affymetrix) and image processing, normalization, and expression analysis were performed with the Affymetrix GCOS ver. 1.4 software. MAS-5 statistical analysis was performed to calculate the signal log ratio (SLR) for each probe set comparison, and the gene expression fold-changes (FCs) between two samples were calculated from the SLR using the following formula: FC=(2SLR). The GCOS 1.2 MAS 5.0 software was used to calculate statistically significant differences in gene expression (P<0.002) between samples.

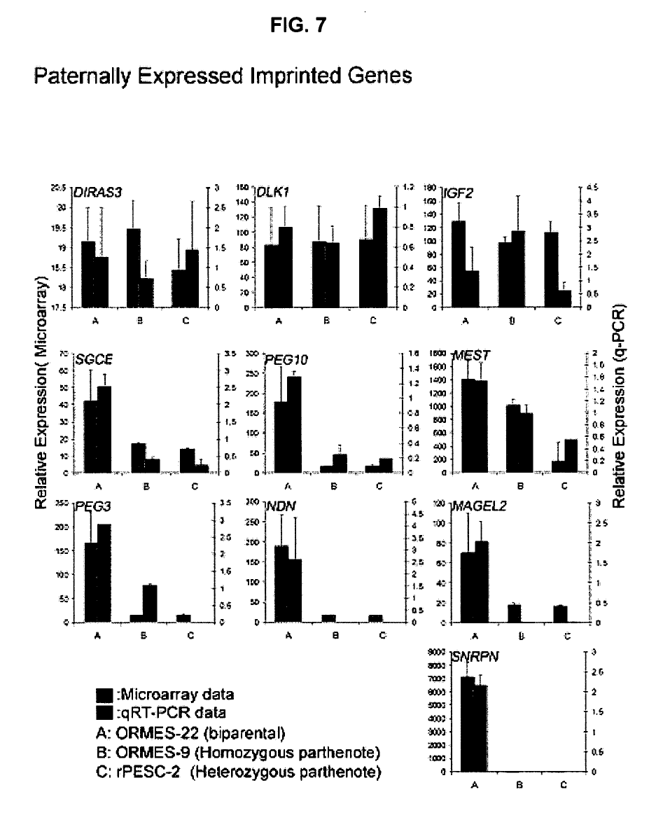

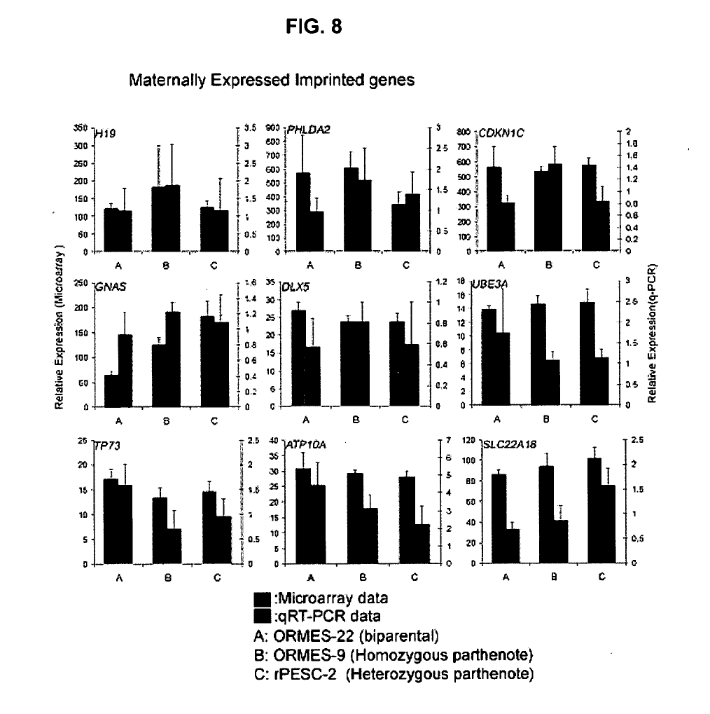

Both Real-time PCR and microarray results confirmed significant expression levels of SGCE and IGF2, suggesting relaxed imprinting at these loci (FIG. 2E and FIG. 7), whereas, mRNA levels were significantly down regulated or absent for PEG10 and PLAGL1. In addition, PEGS, MEST and ZIM2 transcripts were detected in some PESC lines but not in others, whereas transcripts from MAGEL2, MKRN3, SNRPN and NDN were absent in all PESCs, but expressed in bi-parental ESCs (FIGS. 2D, 2E and FIG. 7). Expression levels of maternally expressed imprinted genes in PESCs was similar to fertilization-derived, biparental ESCs (FIG. 2C, 2E and FIG. 8). Thus, monkey PESCs express all imprinted alleles that are normally expressed when inherited from the maternal parent and a portion of imprinted alleles normally expressed only when inherited from the paternal parent.

Epigenetic modifications such as DNA methylation are generally associated with regulation of imprinted gene expression. Parent-of-origin-dependent DNA methylation of CpG dinucleotides, imposed during gametogenesis within so-called imprinting centers (ICs), allows discrimination between maternal and paternal alleles and monoallelic imprinted gene expression. The imprinted expression of adjacent IGF2 and H19 genes is reciprocally controlled by a common IC located upstream of H19, harboring a CpG island that is methylated on the paternal and unmethylated on the maternal chromosome (Mitalipov, S. M. Stem Cells 25, 581 (March, 2007)). In primate MII oocytes, methylation marks are already erased in both alleles within the IGF2/H19 IC whereas both alleles in mature monkey sperm are heavily methylated (Mitalipov, S. M. Stem Cells 25, 581 (March, 2007)). Upon fertilization, typical differentially methylated patterns are restored in the zygote that must be maintained during development. Based on their oocyte parentage, PESCs are not expected to contain methylated alleles at this IC. To test this assumption, the methylation status of this region was analyzed using methylation-sensitive Southern analysis.

### Methylation Analysis by Southern Blot

For Southern analyses of methylation, approximately 4 μg of gDNA from muscle control and ORMES cell lines were digested with EcoNI and the CpG methylation-blocked enzyme BsaHI (New England Biolabs, Ipswich, Mass., USA). The samples were electrophoresed through a 1% agarose gel, and transferred to a nylon membrane. The blot was then hybridized with a probe, whose template was generated by PCR of gDNA from the control tissue using the following primers: Forward: 5′-3′ AGTGCAGGCTCACACATCATAGTC (SEQ ID NO: 101); Reverse: 5′-3′ TAGTCTCTGAGCAAGTAGCGCATC (SEQ ID NO: 102). The probe was produced by random priming (Megaprime DNA Labelling Systems, Amersham BioSciences, GE, Piscataway, N.J.) and labeled with 32^{P}-dCTP (PerkinElmer, Boston, Mass.). The hybridization was carried out at 65° C. overnight in Rapid-hyb buffer (Amersham BioSciences). The blot was washed for 30 minutes in 2×SSC/0.1% SDS and then for 20 mutes in 0.1×SSC/0.1% SDS.

### Methylation Analysis by Bisulfite Sequencing

Methylation analysis was also performed using bisulfite sequencing. Approximately 2 μg of gDNA was modified by busulfite treatment using a CpG Genome Modification Kit (Chemicon International, Temecula, Calif.) according to the manufacturer's protocol. Semi-nested primers for bisulphate sequencing were designed using online software (world wide web at “urogene.org/methprimer”) as previously described (Li, L. C. & Dahiya, R. MethPrimer: designing primers for methylation PCRs. Bioinformatics 18, 1427-31 (2002)). The sequence, annealing temperature, and cycle number of each primer pair were designed as previously reported (Mitalipov, S., Clepper, L., Sritanaudomchai, H., Fujimoto, A. & Wolf, D. Methylation Status of Imprinting Centers for H19/IGF2 and SNURF/SNRPN in Primate Embryonic Stem Cells. Stem Cells 25, 581-8 (2007). PCR reactions were carried out in a 20 μl volume containing 2.5 mM MgCl₂, 2 mM dNTP mix, 0.4 μM each primer, 40 ng template DNA, and 1 U of AccuSure DNA polymerase (Bioline, Randolph, Mass.). Amplicons were electrophoresed through 1.6% TBE agarose gels stained with ethidium bromide and visualized on a UV transilluminator. PCR products were recovered from stained gels (QIAquick Gel Extraction Kit, QIAGEN), ligated with plasmid vector (TOPO TA Cloning Kit for Sequencing, Invitrogen, Carlsbad, Calif.), and cloned according to the manufacturer's protocol. Individual bacterial colonies were transferred to LB/Amp medium and cultured overnight with shaking. Cultures were then processed with Qiaprep Spin Mini-prep Kit (Qiagen) according to the manufacturer's protocol resulting in a single cloned PCR species per plasmid. Individual clones were then sequenced with an ABI 3100 capillary genetic analyzer (Applied Biosystems, Foster City, Calif.) using BigDye terminator sequencing chemistry (Wen, L. Two-step cycle sequencing improves base ambiguities and signal dropouts in DNA sequencing reactions using energy-transfer-based fluorescent dye terminators. Mol Biotechnol 17, 135-42 (2001)). Sequencing results were analyzed using Sequencher software (Gene Codes Corporation, Ann Arbor, Mich.).

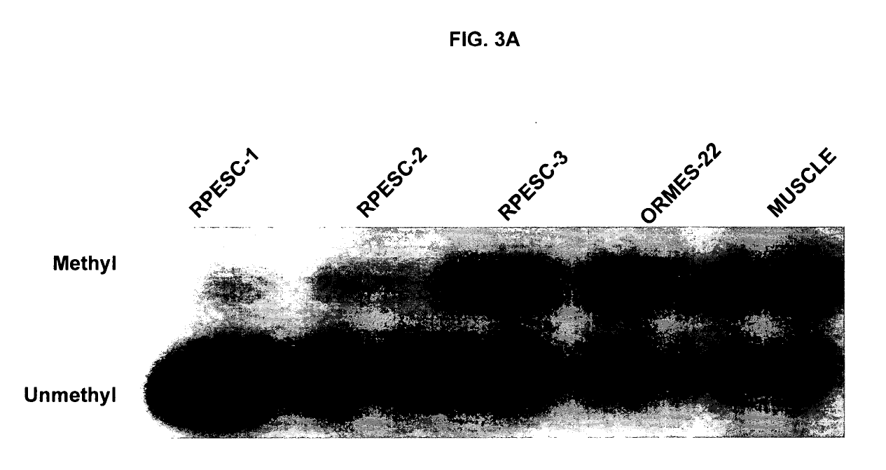

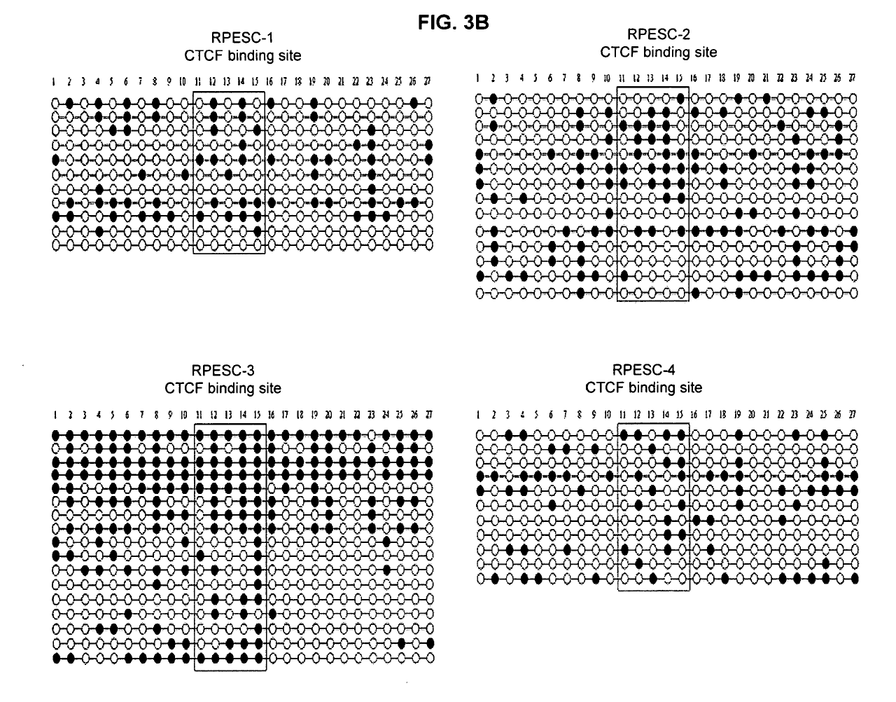

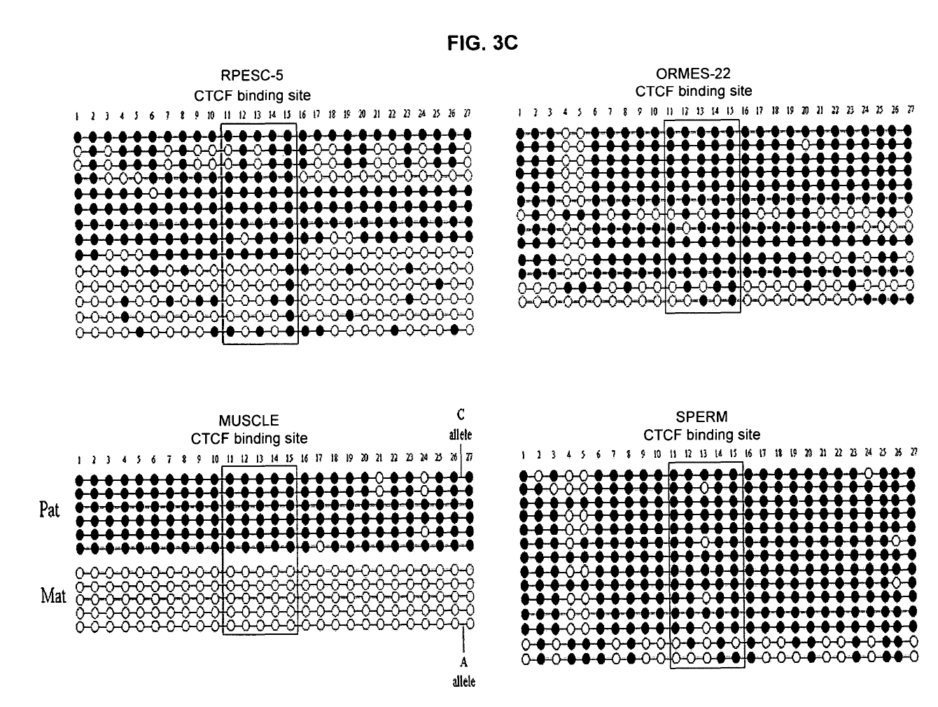

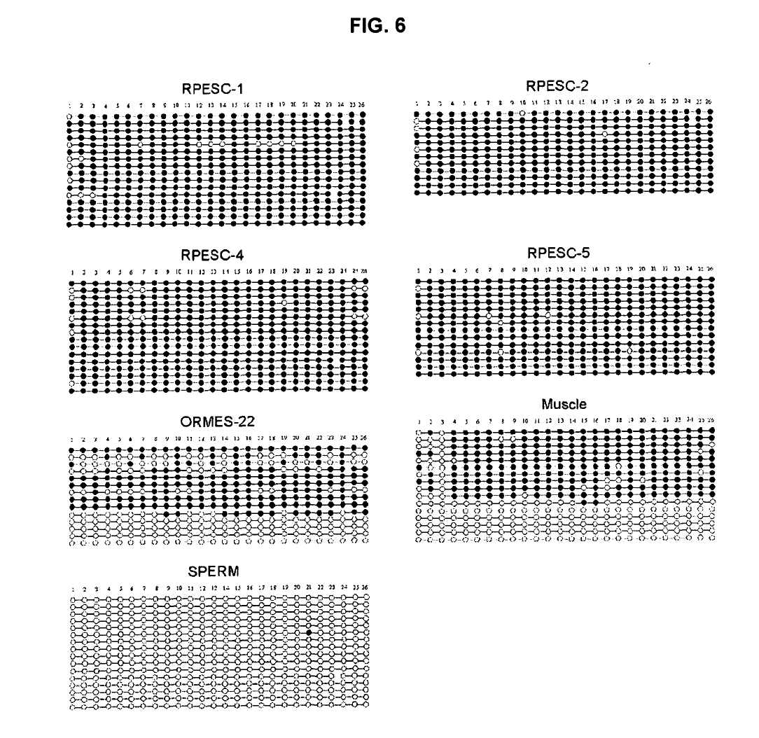

The presence of both cut and uncut fragments following digestion with the methylation-sensitive BsaHI restriction enzyme was observed in control muscle tissue and in bi-parental ORMES-22, reflecting the presence of both methylated and unmethylated alleles (FIG. 3A). Strikingly, PESC lines also showed both uncut (methylated) and cut (unmethylated) fragments, although the level of methylated fragments appeared reduced compared to that observed in bi-parental controls. To further validate these data we performed independent methylation analysis of the IGF2/H19 IC by bisulfite sequencing that confirmed sporadic hypermethylation changes in PESC lines similar to that observed in bi-parental monkey ESCs (FIG. 3B). In contrast, the SNURF/SNRPN IC that is normally methylated on the maternal alleles was completely methylated in PESC cell lines as expected (FIG. 6). Culture conditions used to isolate and propagate PESCs cells may alter normal maintenance of methylation imprints.

### Telomere Length and X-Inactivation

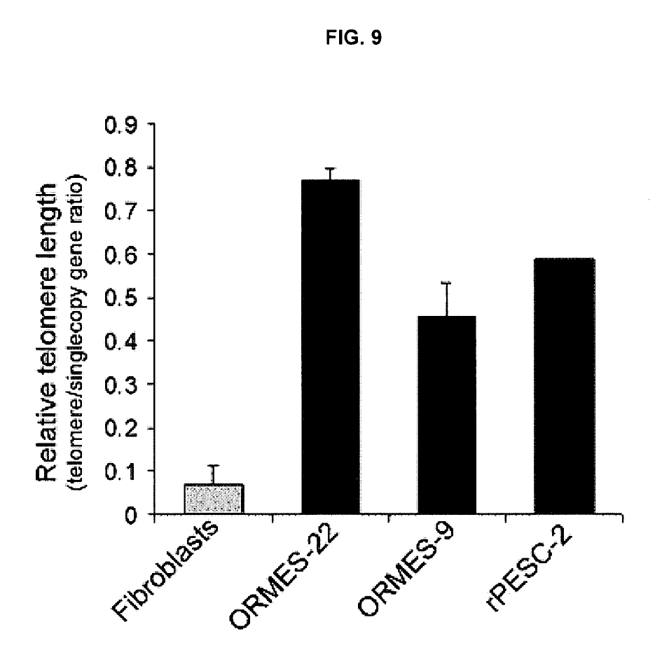

Telomeres are DNA-protein complexes at the ends of eukaryotic chromosomes essential for chromosomal integrity and normal cell growth Telomere DNA is composed of TTAGGG tandem repeats that are progressively incised with each cell division at the rate of 50-150 base pairs per cell division in human cells leading to replicative senescence 19. Maintenance or elongation of telomeres in germ cells, early embryonic and ES cells is sustained by ribonucleoprotein complex telomerase. To determine telomere length in monkey PESCs, a real-time PCR approach was utilized using primers Tel1 and Tel2 for telomeres and 36B4 for acidic ribosomal phosphoprotein PO(RPLPO) used as a single-copy gene reference. Amplification was performed using ABIPrism 7500 sequence detection system (Applied Biosystems) under following conditions: for Tel1 and Tel2 primers—30 cycles at 95° C. for 15 sec and 54° C. for 2 min; for 36B4 primers—30 cycles at 95° C. for 15 sec and 58° C. for 1 min. To determine the cycle threshold (Ct) value, 2 separate PCR runs were performed for each sample and primer pair. For each run a standard curve was generated using a reference genomic DNA isolated from IVF-derived ESCs diluted to 0.06 to 40 ng per well (5 fold dilution). Calculation of the relative telomere/single copy gene ratio (T/S value) and statistical analysis with SDS v. 1.1 software (Applied Biosystems) was used to determine standard curve and Ct values. A point on the standard curve at a concentration corresponding to the average DNA concentration of the samples was used as a calibrator. The mean T/S value of skin fibroblasts and ESCs were compared and plotted against each sample. The results clearly demonstrated a significant elongation of telomere length in parthenote-derived ESC lines (RPES-2 and ORMES-9) relative to skin fibroblasts (FIG. 9). Telomere length in parthenotes was comparable to IVF-derived ORMES-22. These data indicate an efficient reprogramming and restoration of replicative capacity of donor somatic cells to embryonic levels after primate SCNT.

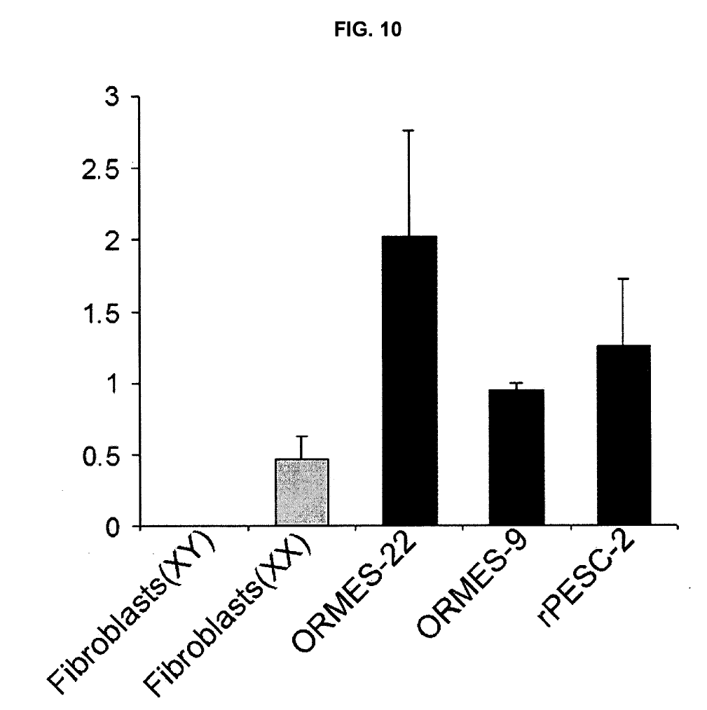

Dosage compensation in female mammals is achieved by epigenetic processes that silence gene expression from one X chromosome, a process known as X-inactivation. In the mouse, both X chromosomes are presumed to be active during preimplantation embryonic development and undifferentiated mouse ESCs do not display an inactive X. Random X-inactivation occurs in differentiating mouse ESCs upon expression of the xist gene from the chromosome to be inactivated, thereby allowing this non-coding transcript to associate with the chromosome of origin and silence gene transcription. The timing and developmental regulation of X-inactivation in primates is unclear. However, in contrast to the mouse, the majority of examined undifferentiated human ESC lines show X-inactivation suggesting inactivation occurs early in primate development. The X-inactivation status of male (XY) fibroblasts, ORMES-22 (biparental), ORMES-9 (homozygous parthenote) and rPESC-2 (heterozygous parthenote) was assessed by measuring the level of XIST expression by qPCR. All tested female samples including ORMES-22, ORMES-9 and IVF-derived ORMES-22 displayed strong XIST expression (FIG. 10). In contrast, XIST transcripts were not detected in male-derived fibroblasts. These data indicate that epigenetic marks regulating X-inactivation are faithfully recapitulated in primate parthenogenetic ESCs.

### Histocompatibility in Primate PESCs

Recently mouse PESC lines were described with restored heterozygous maternal MHC haplotypes. Cells with heterozygous maternal MHC haplotypes can be used as a source of histocompatible tissues for autologous transplantation into the oocyte donor without rejection. To investigate if primate PESCs may re-establish heterozygosity within the MHC region, a comprehensive genotyping of PESC lines and oocyte donors was performed within 15 polymorphic microsatellite markers mapped within or near the MHC locus (Penedo, M. C. et al., Immunogenetics, “Microsatellite typing of the rhesus macaque MHC region.” Immunogenetics,” 57(3-4): 198-209. (Apr. 5, 2005)). Microsatellite genotyping provides an alternative method for the rapid and accurate prediction of immunocompatibility and is widely used for MHC analysis in humans for tissue matching and donor screening (Carrington, M. et al. Hum Immunol 51, 106 (December, 1996); Foissac A. et al., Transplant Proc 33, 491 (February-March, 2001)).

STR sequencing and comparisons with the complete Rhesus monkey MHC genomic map indicated exact positioning of these markers within the 5.3 Mb MHC region on chromosome 6, with D6S291 located at the centromeric end and D6S276/D6S1691 at the telomeric end (Penedo, M. C. et al., Immunogenetics (Apr. 5, 2005)). Comparisons of genotypes in oocyte donors and their PESC lines using the methods described above revealed that in 4 cell lines, STR alleles were organized together into strong haplotype blocks during meiotic recombination, resulting in either complete homozygosity (RPESC-2, Table 7) or heterozygosity (RPESC-1, -4, and -5). RPESC-1, -4, and -5 showed recombination upstream of the MHC region that fully restored heterozygosity and resulted in genotypes across the MHC region that were identical to the oocyte donors (Table 7). Therefore, these cell lines can be considered isogenic and should not be rejected upon autologous transplantation. In contrast to the strong genetic linkage between MHC haplotypes during meiotic recombination observed in RPESC-1, -2, -4, and -5, RPESC-3 displayed a recombination event within the MHC region. This important observation suggests the presence of a single meiotic crossover between MICA and 246K06 loci (Table 7). The Cyno-1 cell line was homozygous within all 15 analyzed MHC-linked STRs.

Table 7. Histocompatibility analysis of monkey egg donors and their PESC lines based on WIC-linked STR analysis (heterozygous loci are shown in gray)

### Example 2

### Removal of Immortalized Primate Parthenotes or Cells Derived from Primate Parthenotes

If desired, the primate parthenotes described herein or cells (such as multipotent stem cells or transplantable cells) derived from them can be tested to make sure they are not immortalized before they are used in a pharmaceutical composition, therapeutic method, or therapeutic kit of the invention. In particular, standard methods can be used to determine the number of cell divisions that the cell is capable of undergoing. In some embodiments, cells capable of undergoing at least about any of 1.5-fold, 2-fold, 3-fold, 4-fold, or 5-fold more cell divisions than a naturally-occurring control cell of the same type, genus, and species as the immortalized cell are omitted from the pharmaceutical compositions, therapeutic methods, and therapeutic kits of the invention. Such cells may have, for example, two copies of a recessive oncogene that results in abnormal cell growth.

### Example 3

### Exemplary Production and Use of Differentiated Cells

Primate parthenotes are produced according to the methods disclosed herein, and pluripotent embryonic stem cells are produced from these parenotes, as described above. In order to differentiate the human ES cells into neurons, the ES cells are cultured using protocols described in PCT Publication No. WO 2005/017131, which is incorporated by reference herein. Specifically medium comprising the parthenote is replaced with a medium including one or more of insulin, transferrin or selenium, such as a medium including insulin, transferrin and selenium. In one example, the medium is ITS medium. ITSFn medium includes DMEM and F12 in a ratio between 0.1:1 and 10:1 supplemented with between about 1 ng/ml to about 10 ng/ml insulin, about 20 nM to about 40 nM selenium chloride, about 40 ng/ml to about 60 ng/ml transferrin and between about 1 ng/ml to 10 ng/ml fibronectin. The cells are incubated in this medium for between about 2 to about 20 days at a temperature between about 35° C. and about 40° C. The media is changed about every 3-5 days, such as about every four days. In one example, the cells are incubated at 37° C. under between about 1% and 10% CO₂ atmosphere, or between about 5% and 10% CO₂ or under about 5% CO₂.

Following growth in the medium including one or more of insulin, transferrin, and selenium, the cells are grown in a medium including one or more of insulin, transferrin, putrescine, selenite, and/or progesterone. In one example, the medium is N2 medium. This medium includes DMEM/F12 at a ratio of 1:1, and insulin, transferrin, progesterone, putrescine, and selenium.

Neuronal cells are produced in order to deliver the cells, or molecules expressed by these cells, to the brain for diagnosis, treatment or prevention of disorders or diseases of the CNS, brain, and/or spinal cord. These disorders can be neurologic or psychiatric disorders. These disorders or diseases include brain diseases such as Alzheimer's disease, Parkinson's disease, Lewy body dementia, multiple sclerosis, epilepsy, cerebellar ataxia, progressive supranuclear palsy, amyotrophic lateral sclerosis, affective disorders, anxiety disorders, obsessive compulsive disorders, personality disorders, attention deficit disorder, attention deficit hyperactivity disorder, Tourette Syndrome, Tay Sachs, Nieman Pick, and other lipid storage and genetic brain diseases and/or schizophrenia. The method can also be employed in subjects suffering from or at risk for nerve damage from cerebrovascular disorders such as stroke in the brain or spinal cord, from CNS infections including meningitis and HIV, from tumors of the brain and spinal cord, or from a prior disease. The method can also be employed to deliver agents to counter CNS disorders resulting from ordinary aging (e.g., insomnia or loss of the general chemical sense), brain injury, or spinal cord injury.

The foregoing examples and detailed description are offered by way of illustration and not by way of limitation. All publications, patent applications, patents, and sequences from GenkBank and other databases cited in this specification are herein incorporated by reference as if each individual publication, patent application, or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention.

1. A library comprising an isolated or purified primate parthenote or an isolated or purified cell derived from the primate parthenote, wherein the parthenote or cell comprises a set of heterozygous MHC alleles and a set of heterozygous alleles for a gene that does not encode an MHC molecule.

2. The library of claim 1, further comprising a second primate parthenote or cell, wherein the second parthenote or cell comprises a set of heterozygous MHC alleles and a set of homozygous alleles for a gene that does not encode an MHC molecule.

3. The library of claim 1, further comprising a third primate additional parthenote or cell, wherein the third parthenote or cell comprises a set of homozygous MHC alleles and a set of homozygous alleles for a gene that does not encode an MHC molecule.

4. The library of claim 1, wherein the parthenote or cell comprises a set of heterozygous MHC alleles and a set of heterozygous alleles for a gene that does not encode an MHC molecule, wherein the second parthenote or cell comprises a set of heterozygous MHC alleles and a set of homozygous alleles for a gene that does not encode an MHC molecule, and wherein the third parthenote or cell comprises a set of homozygous MHC alleles and a set of homozygous alleles for a gene that does not encode an MHC molecule.

5. (canceled)

6. (canceled)

7. (canceled)

8. (canceled)

9. The library of claim 1, wherein the first parthenote or cell and/or the second parthenote or cell is heterozygous for HLA-A, HLA-B, HLA-DR, or any two or more of the foregoing alleles.

10. The library of claim 6, wherein the first parthenote or cell and/or the second parthenote or cell is heterozygous for HLA-A, HLA-B, and HLA-DR alleles.

11. The library of claim 1, wherein the first parthenote or cell and/or the second parthenote or cell is heterozygous for one or more of the following MHC-linked microsatellite loci: D6S291, D6S2741, D6S2876, 9P06, DRA, MICA, 246K06, 162B17A, 162B17B, 151L13, MOGCA, 268P23, 222I18, D6S276, and D6S1691.

12. The library of claim 1, wherein at least one parthenote or cell expresses a paternally expressed imprinted gene.

13. A method for generating a primate partenote, comprising
isolating a primate oocyte in metaphase II;
incubating the primate oocyte in a first medium comprising about 1 to about 10 μM ionomycin and a first concentration of serum albumin for about 1 to about 10 minutes;
incubating the primate oocyte in a second medium comprising a second concentration of serum albumin and 6-dimethylaminopurine, wherein the ratio of the first concentration of serum album to the second concentration of serum albumin is 1:20 to 1:40, and wherein the first concentration of serum albumin is between about 0.1 mg./ml to about 10 mg/ml; and
culturing the primate oocyte in to form an embryo in vitro.

14. The method of claim 13, comprising culturing the oocyte in about 5 μM ionomycin.

15. The method of claim 13, wherein culturing the oocyte in a first medium comprising ionomycin comprises culturing in ionomycin for about 5 minutes.

16. The method of claim 13, wherein culturing in the oocyte in a second medium comprising 6-dimethylaminopurine comprises culturing the oocyte for about 5 minutes.

17. The method of claim 13, wherein the first concentration of bovine serum albumin is about 1 mg/ml.

18. The method of claim 13, wherein the ratio of the first concentration of bovine serum albumin to the second concentration of bovine serum albumin is 1:30.

19. The method of claim 13, wherein the second medium comprises one of (1) about 2 mM 6-dimethlyaminopurine or (2) about 4 μg/ml to about 6 μg/ml cytoclasin B.

20. The method of claim 19, wherein the second medium comprises about 4 μg/ml to about 6 μg/ml cytoclasin B and about 25 μg/ml to about 75 μg/ml roscovitine.

21. The method of claim 19, wherein the second medium comprises about 4 μg/ml to about 6 μg/ml cytoclasin B and about 5 μg/ml to about 8 μg/ml cycloheximide.

22. The method of claim 19, wherein the second medium comprises about 5 μg/ml cytoclasin B and about 7.5 μg/ml cycloheximide.

23. The method of claim 13, wherein culturing the oocyte to form an embryo in vitro comprises culturing the oocyte in about 5% O₂.

24. The method of claim 13, wherein culturing the oocyte to form an embryo comprises culturing the oocyte in third medium comprising 6-dimethlyaminopurine.

25. The method of claim 24, wherein the third medium comprises about 2 mM 6-dimethylaminopurine.

26. The method of claim 13, wherein the first and the second medium are TALP/HEPES medium.

27. The method of claim 27, wherein the third medium is TALP/HEPEPS medium.

28. The method of claim 13, wherein the embryo is a blastocyst.

29. The method of claim 13, wherein the embryo is a 4-cell or an 8-cell embryo.

30. A method of producing a cell type of interest, comprising
producing a primate parthenote according to the method of claim 13; and
culturing the primate oocyte, thereby producing the cell type of interest.

31. The method of claim 30, wherein the cell type of interest is a pluripotent embryonic stem cell.

32. The method of claim 13, wherein the oocyte is a monkey oocyte.

33. The method of claim 13, wherein the oocyte is a human oocyte.

34. (canceled)

35. (canceled)

36. (canceled)

37. (canceled)
